SEQUENCE LISTING (162 sequences)

SEQ ID NO: 1 - DNA, 31 bp, Artificial Sequence
  Description of Artificial Sequence Synthetic primer
  modified_base (16)..(16): Phosphorothioate-T base
  modified_base (17)..(17): Phosphorothioate-G base
tgctggagtgaacgctgggccgagcgcaaag

SEQ ID NO: 2 - DNA, 23 bp, Artificial Sequence
  Description of Artificial Sequence Synthetic primer
  modified_base (16)..(16): Phosphorothioate-C base
  modified_base (17)..(17): Phosphorothioate-G base
gcaagaaaactatcccgaccgcc

SEQ ID NO: 3 - DNA, 25 bp, Artificial Sequence
  Description of Artificial Sequence Synthetic primer
  modified_base (16)..(16): Phosphorothioate-G base
  modified_base (17)..(17): Phosphorothioate-G base
ggatagttttcttgcggccctaatc

SEQ ID NO: 4 - DNA, 22 bp, Artificial Sequence
  Description of Artificial Sequence Synthetic primer
  modified_base (16)..(16): Phosphorothioate-G base
  modified_base (17)..(17): Phosphorothioate-G base
cgtctgggactgggtggatcag

SEQ ID NO: 5 - DNA, 22 bp, Artificial Sequence
  Description of Artificial Sequence Synthetic primer
  modified_base (16)..(16): Phosphorothioate-A base
  modified_base (17)..(17): Phosphorothioate-A base
acccagtcccagacgaagccgc

SEQ ID NO: 6 - DNA, 27 bp, Artificial Sequence
  Description of Artificial Sequence Synthetic primer
  modified_base (16)..(16): Phosphorothioate-T base
  modified_base (17)..(17): Phosphorothioate-T base
cagatgtgcggcgagttgcgtgactac

SEQ ID NO: 7 - DNA, 24 bp, Artificial Sequence
  Description of Artificial Sequence Synthetic primer
  modified_base (16)..(16): Phosphorothioate-A base
  modified_base (17)..(17): Phosphorothioate-A base
ctcgccgcacatctgaacttcagc

SEQ ID NO: 8 - DNA, 24 bp, Artificial Sequence
  Description of Artificial Sequence Synthetic primer
  modified_base (16)..(16): Phosphorothioate-A base
  modified_base (17)..(17): Phosphorothioate-T base
cgcagtggaagatagatctgattg

SEQ ID NO: 9 - DNA, 22 bp, Artificial Sequence
  Description of Artificial Sequence Synthetic primer
  modified_base (16)..(16): Phosphorothioate-A base
  modified_base (17)..(17): Phosphorothioate-G base
ctatcttccactgcgagttgaa

SEQ ID NO: 10 - DNA, 25 bp, Artificial Sequence
  Description of Artificial Sequence Synthetic primer
  modified_base (16)..(16): Phosphorothioate-G base
  modified_base (17)..(17): Phosphorothioate-T base
agtgcagttggtggagttgttgatg

SEQ ID NO: 11 - DNA, 24 bp, Artificial Sequence
  Description of Artificial Sequence Synthetic primer
  modified_base (16)..(16): Phosphorothioate-A base
  modified_base (17)..(17): Phosphorothioate-G base
tccaccaactgcactaggagattg

SEQ ID NO: 12 - DNA, 27 bp, Artificial Sequence
  Description of Artificial Sequence Synthetic primer
  modified_base (16)..(16): Phosphorothioate-A base
  modified_base (17)..(17): Phosphorothioate-A base
agcaaggtgagattgaaactaggattg

SEQ ID NO: 13 - DNA, 25 bp, Artificial Sequence
  Description of Artificial Sequence Synthetic primer
  modified_base (16)..(16): Phosphorothioate-G base
  modified_base (17)..(17): Phosphorothioate-T base
caatctcaccttgctgtgctttagc

SEQ ID NO: 14 - DNA, 28 bp, Artificial Sequence
  Description of Artificial Sequence Synthetic primer
  modified_base (16)..(16): Phosphorothioate-T base
  modified_base (17)..(17): Phosphorothioate-G base
tcttgccctagcagttggtcataccaac

SEQ ID NO: 15 - DNA, 30 bp, Artificial Sequence
  Description of Artificial Sequence Synthetic primer
  modified_base (16)..(16): Phosphorothioate-A base
  modified_base (17)..(17): Phosphorothioate-C base
actgctagggcaagaaccaccaccaaatag

SEQ ID NO: 16 - DNA, 28 bp, Artificial Sequence
  Description of Artificial Sequence Synthetic primer
  modified_base (16)..(16): Phosphorothioate-C base
  modified_base (17)..(17): Phosphorothioate-A base
ctttagatggtgagacagtttatgcagg

SEQ ID NO: 17 - DNA, 24 bp, Artificial Sequence
  Description of Artificial Sequence Synthetic primer
  modified_base (16)..(16): Phosphorothioate-T base
  modified_base (17)..(17): Phosphorothioate-A base
tctcaccatctaaagtaacgatcc

SEQ ID NO: 18 - DNA, 23 bp, Artificial Sequence
  Description of Artificial Sequence Synthetic primer
  modified_base (16)..(16): Phosphorothioate-A base
  modified_base (17)..(17): Phosphorothioate-A base
ctgttgggttagatcaaatggcg

SEQ ID NO: 19 - DNA, 22 bp, Artificial Sequence
  Description of Artificial Sequence Synthetic primer
  modified_base (16)..(16): Phosphorothioate-T base
  modified_base (17)..(17): Phosphorothioate-A base
gatctaacccaacagtaggttc

SEQ ID NO: 20 - DNA, 28 bp, Artificial Sequence
  Description of Artificial Sequence Synthetic primer
  modified_base (16)..(16): Phosphorothioate-T base
  modified_base (17)..(17): Phosphorothioate-C base
cacatgcctcccttttccacttttattg

SEQ ID NO: 21 - DNA, 24 bp, Artificial Sequence
  Description of Artificial Sequence Synthetic primer
  modified_base (16)..(16): Phosphorothioate-A base
  modified_base (17)..(17): Phosphorothioate-G base
aaagggaggcatgtgagcaaaagg

SEQ ID NO: 22 - DNA, 28 bp, Artificial Sequence
  Description of Artificial Sequence Synthetic primer
  modified_base (16)..(16): Phosphorothioate-C base
  modified_base (17)..(17): Phosphorothioate-G base
gcccagcgttcaggccgcgatatcaccc

SEQ ID NO: 23 - DNA, 34 bp, Artificial Sequence
  Description of Artificial Sequence Synthetic primer
  modified_base (16)..(16): Phosphorothioate-A base
  modified_base (17)..(17): Phosphorothioate-A base
ggcctaaaagactctaacaaaatagcaaatttcg

SEQ ID NO: 24 - DNA, 21 bp, Artificial Sequence
  Description of Artificial Sequence Synthetic primer
  modified_base (16)..(16): Phosphorothioate-C base
  modified_base (17)..(17): Phosphorothioate-A base
cccattaggccatttcagcag

SEQ ID NO: 25 - DNA, 34 bp, Artificial Sequence
  Description of Artificial Sequence Synthetic primer
  modified_base (16)..(16): Phosphorothioate-T base
  modified_base (17)..(17): Phosphorothioate-T base
aaatggcctaatgggttacgatgctttgttcttg

SEQ ID NO: 26 - DNA, 37 bp, Artificial Sequence
  Description of Artificial Sequence Synthetic primer
  modified_base (16)..(16): Phosphorothioate-T base
  modified_base (17)..(17): Phosphorothioate-G base
acctctccaataatttgttccaagtaaccatcttcac

SEQ ID NO: 27 - DNA, 31 bp, Artificial Sequence
  Description of Artificial Sequence Synthetic primer
  modified_base (16)..(16): Phosphorothioate-T base
  modified_base (17)..(17): Phosphorothioate-G base
aattattggagaggttgtgttgctgaaggtg

SEQ ID NO: 28 - DNA, 27 bp, Artificial Sequence
  Description of Artificial Sequence Synthetic primer
  modified_base (16)..(16): Phosphorothioate-C base
  modified_base (17)..(17): Phosphorothioate-C base
gcttcacccacaaagccaatctagcac

SEQ ID NO: 29 - DNA, 29 bp, Artificial Sequence
  Description of Artificial Sequence Synthetic primer
  modified_base (16)..(16): Phosphorothioate-T base
  modified_base (17)..(17): Phosphorothioate-G base
ctttgtgggtgaagctgatagaggtgatg

SEQ ID NO: 30 - DNA, 29 bp, Artificial Sequence
  Description of Artificial Sequence Synthetic primer
  modified_base (16)..(16): Phosphorothioate-A base
  modified_base (17)..(17): Phosphorothioate-C base
tctggtcatctctcaacaacaaatcaccc

SEQ ID NO: 31 - DNA, 33 bp, Artificial Sequence
  Description of Artificial Sequence Synthetic primer
  modified_base (16)..(16): Phosphorothioate-T base
  modified_base (17)..(17): Phosphorothioate-T base
tgagagatgaccagatttgggtgctaaattgcc

SEQ ID NO: 32 - DNA, 29 bp, Artificial Sequence
  Description of Artificial Sequence Synthetic primer
  modified_base (16)..(16): Phosphorothioate-T base
  modified_base (17)..(17): Phosphorothioate-C base
gttcagcagttctcttcttctatcaccag

SEQ ID NO: 33 - DNA, 28 bp, Artificial Sequence
  Description of Artificial Sequence Synthetic primer
  modified_base (16)..(16): Phosphorothioate-A base
  modified_base (17)..(17): Phosphorothioate-A base
agagaactgctgaacaattacaattggc

SEQ ID NO: 34 - DNA, 27 bp, Artificial Sequence
  Description of Artificial Sequence Synthetic primer
  modified_base (16)..(16): Phosphorothioate-C base
  modified_base (17)..(17): Phosphorothioate-A base
ttctagccaaggttccaacatggaggc

SEQ ID NO: 35 - DNA, 33 bp, Artificial Sequence
  Description of Artificial Sequence Synthetic primer
  modified_base (16)..(16): Phosphorothioate-G base
  modified_base (17)..(17): Phosphorothioate-A base
gaaccttggctagaagatgtgaaagattattgg

SEQ ID NO: 36 - DNA, 24 bp, Artificial Sequence
  Description of Artificial Sequence Synthetic primer
  modified_base (16)..(16): Phosphorothioate-C base
  modified_base (17)..(17): Phosphorothioate-A base
aaccagaaaggctctcatagtagg

SEQ ID NO: 37 - DNA, 28 bp, Artificial Sequence
  Description of Artificial Sequence Synthetic primer
  modified_base (16)..(16): Phosphorothioate-C base
  modified_base (17)..(17): Phosphorothioate-T base
agagcctttctggttctccatctttgac

SEQ ID NO: 38 - DNA, 30 bp, Artificial Sequence
  Description of Artificial Sequence Synthetic primer
  modified_base (16)..(16): Phosphorothioate-G base
  modified_base (17)..(17): Phosphorothioate-A base
ctcaaagccgaatctgatggcaataccttg

SEQ ID NO: 39 - DNA, 30 bp, Artificial Sequence
  Description of Artificial Sequence Synthetic primer
  modified_base (16)..(16): Phosphorothioate-A base
  modified_base (17)..(17): Phosphorothioate-G base
agattcggctttgagagataagtgtagatc

SEQ ID NO: 40 - DNA, 28 bp, Artificial Sequence
  Description of Artificial Sequence Synthetic primer
  modified_base (16)..(16): Phosphorothioate-C base
  modified_base (17)..(17): Phosphorothioate-C base
ccaataagacagtaaccagaagtcaatt

SEQ ID NO: 41 - DNA, 29 bp, Artificial Sequence
  Description of Artificial Sequence Synthetic primer
  modified_base (16)..(16): Phosphorothioate-T base
  modified_base (17)..(17): Phosphorothioate-C base
ttactgtcttattggtcaccaatgttgcc

SEQ ID NO: 42 - DNA, 29 bp, Artificial Sequence
  Description of Artificial Sequence Synthetic primer
  modified_base (16)..(16): Phosphorothioate-C base
  modified_base (17)..(17): Phosphorothioate-C base
cacatgctatagaacccgaacgaccgagc

SEQ ID NO: 43 - DNA, 29 bp, Artificial Sequence
  Description of Artificial Sequence Synthetic primer
  modified_base (16)..(16): Phosphorothioate-A base
  modified_base (17)..(17): Phosphorothioate-G base
gttctatagcatgtgagcaaaaggccagc

SEQ ID NO: 44 - DNA, 30 bp, Artificial Sequence
  Description of Artificial Sequence Synthetic primer
  modified_base (16)..(16): Phosphorothioate-G base
  modified_base (17)..(17): Phosphorothioate-C base
agagtcttttaggccgcgatatcaccccta

SEQ ID NO: 45 - DNA, 21 bp, Artificial Sequence
  Description of Artificial Sequence Synthetic primer
  modified_base (13)..(13): Phosphorothioate-C base
  modified_base (14)..(14): Phosphorothioate-C base
cgcggaacctgacccccgaac

SEQ ID NO: 46 - DNA, 22 bp, Artificial Sequence
  Description of Artificial Sequence Synthetic primer
  modified_base (13)..(13): Phosphorothioate-C base
  modified_base (14)..(14): Phosphorothioate-T base
cagtccgtgagcctggcacagc

SEQ ID NO: 47 - DNA, 19 bp, Artificial Sequence
  Description of Artificial Sequence Synthetic primer
  modified_base (13)..(13): Phosphorothioate-A base
  modified_base (14)..(14): Phosphorothioate-C base
gctcacggactgacccccg

SEQ ID NO: 48 - DNA, 20 bp, Artificial Sequence
  Description of Artificial Sequence Synthetic primer
  modified_base (13)..(13): Phosphorothioate-C base
  modified_base (14)..(14): Phosphorothioate-C base
tcagcccgtgagcctggcac

SEQ ID NO: 49 - DNA, 18 bp, Artificial Sequence
  Description of Artificial Sequence Synthetic primer
  modified_base (13)..(13): Phosphorothioate-C base
  modified_base (14)..(14): Phosphorothioate-C base
ctcacgggctgacccccg

SEQ ID NO: 50 - DNA, 22 bp, Artificial Sequence
  Description of Artificial Sequence Synthetic primer
  modified_base (13)..(13): Phosphorothioate-C base
  modified_base (14)..(14): Phosphorothioate-G base
cgggggtcaaaccgtgagcctg

SEQ ID NO: 51 - DNA, 18 bp, Artificial Sequence
  Description of Artificial Sequence Synthetic primer
  modified_base (13)..(13): Phosphorothioate-A base
  modified_base (14)..(14): Phosphorothioate-A base
gtttgacccccgaacagg

SEQ ID NO: 52 - DNA, 20 bp, Artificial Sequence
  Description of Artificial Sequence Synthetic primer
  modified_base (13)..(13): Phosphorothioate-A base
  modified_base (14)..(14): Phosphorothioate-G base
tgtgaggccgtgagcctggc

SEQ ID NO: 53 - DNA, 25 bp, Artificial Sequence
  Description of Artificial Sequence Synthetic primer
  modified_base (13)..(13): Phosphorothioate-T base
  modified_base (14)..(14): Phosphorothioate-G base
cacggcctcacatgtgagcaaaagg

SEQ ID NO: 54 - DNA, 24 bp, Artificial Sequence
  Description of Artificial Sequence Synthetic primer
  modified_base (13)..(13): Phosphorothioate-A base
  modified_base (14)..(14): Phosphorothioate-T base
tcaggttccgcgatatcaccccta

SEQ ID NO: 55 - DNA, 10 bp, Artificial Sequence
  Description of Artificial Sequence Synthetic oligonucleotide
agataaacat

SEQ ID NO: 56 - DNA, 30 bp, Artificial Sequence
  Description of Artificial Sequence Synthetic oligonucleotide
taggccatttcagcagaaatatctggcaag

SEQ ID NO: 57 - DNA, 27 bp, Artificial Sequence
  Description of Artificial Sequence Synthetic oligonucleotide
tcttattggtcaccaatgttgccagac

SEQ ID NO: 58 - DNA, 29 bp, Artificial Sequence
  Description of Artificial Sequence Synthetic oligonucleotide
ttatctcttagcagcaaaaacagcatctg

SEQ ID NO: 59 - DNA, 32 bp, Artificial Sequence
  Description of Artificial Sequence Synthetic oligonucleotide
ccaaagcttcaggggataacgcaggaaagaac

SEQ ID NO: 60 - DNA, 36 bp, Artificial Sequence
  Description of Artificial Sequence Synthetic oligonucleotide
ttgaagctttctgattatcaaccggggtggagcttc

SEQ ID NO: 61 - DNA, 40 bp, Artificial Sequence
  Description of Artificial Sequence Synthetic oligonucleotide
gaaatttgctattttgttagagtcttttacaccatttgtc

SEQ ID NO: 62 - DNA, 24 bp, Artificial Sequence
  Description of Artificial Sequence Synthetic oligonucleotide
aaaaaatgtagaggtcgagtttag

SEQ ID NO: 63 - DNA, 22 bp, Artificial Sequence
  Description of Artificial Sequence Synthetic oligonucleotide
taaaagactctaacaaaatagc

SEQ ID NO: 64 - DNA, 21 bp, Artificial Sequence
  Description of Artificial Sequence Synthetic oligonucleotide
atgggttacgatgctttgttc

SEQ ID NO: 65 - DNA, 22 bp, Artificial Sequence
  Description of Artificial Sequence Synthetic oligonucleotide
taatttgttccaagtaaccatc

SEQ ID NO: 66 - DNA, 20 bp, Artificial Sequence
  Description of Artificial Sequence Synthetic oligonucleotide
ttggagaggttgtgttgctg

SEQ ID NO: 67 - DNA, 20 bp, Artificial Sequence
  Description of Artificial Sequence Synthetic oligonucleotide
ccacaaagccaatctagcac

SEQ ID NO: 68 - DNA, 21 bp, Artificial Sequence
  Description of Artificial Sequence Synthetic oligonucleotide
gtgaagctgatagaggtgatg

SEQ ID NO: 69 - DNA, 20 bp, Artificial Sequence
  Description of Artificial Sequence Synthetic oligonucleotide
tcatctctcaacaacaaatc

SEQ ID NO: 70 - DNA, 21 bp, Artificial Sequence
  Description of Artificial Sequence Synthetic oligonucleotide
ccagatttgggtgctaaattg

SEQ ID NO: 71 - DNA, 22 bp, Artificial Sequence
  Description of Artificial Sequence Synthetic oligonucleotide
tctcttcttctatcaccagaac

SEQ ID NO: 72 - DNA, 21 bp, Artificial Sequence
  Description of Artificial Sequence Synthetic oligonucleotide
actgctgaacaattacaattg

SEQ ID NO: 73 - DNA, 20 bp, Artificial Sequence
  Description of Artificial Sequence Synthetic oligonucleotide
ggttccaacatggaggcttg

SEQ ID NO: 74 - DNA, 20 bp, Artificial Sequence
  Description of Artificial Sequence Synthetic oligonucleotide
ttggctagaagatgtgaaag

SEQ ID NO: 75 - DNA, 20 bp, Artificial Sequence
  Description of Artificial Sequence Synthetic oligonucleotide
aaggctctcatagtaggttc

SEQ ID NO: 76 - DNA, 20 bp, Artificial Sequence
  Description of Artificial Sequence Synthetic oligonucleotide
tctggttctccatctttgac

SEQ ID NO: 77 - DNA, 22 bp, Artificial Sequence
  Description of Artificial Sequence Synthetic oligonucleotide
ctttcgaatctgatggcaatac

SEQ ID NO: 78 - DNA, 20 bp, Artificial Sequence
  Description of Artificial Sequence Synthetic oligonucleotide
gctttgagagataagtgtag

SEQ ID NO: 79 - DNA, 20 bp, Artificial Sequence
  Description of Artificial Sequence Synthetic oligonucleotide
cagtaaccagaagtcaattg

SEQ ID NO: 80 - DNA, 41 bp, Artificial Sequence
  Description of Artificial Sequence Synthetic oligonucleotide
gagtaaacttggtctgacagtcagaagaactcgtcaagaag

SEQ ID NO: 81 - DNA, 41 bp, Artificial Sequence
  Description of Artificial Sequence Synthetic oligonucleotide
cttcttgacgagttcttctgactgtcagaccaagtttactc

SEQ ID NO: 82 - DNA, 19 bp, Artificial Sequence
  Description of Artificial Sequence Synthetic oligonucleotide
gaaaagtgccacctgacgt

SEQ ID NO: 83 - DNA, 19 bp, Artificial Sequence
  Description of Artificial Sequence Synthetic oligonucleotide
tcaagaaggcgatagaagg

SEQ ID NO: 84 - DNA, 20 bp, Artificial Sequence
  Description of Artificial Sequence Synthetic oligonucleotide
ttttttctgcgcgtaatctg

SEQ ID NO: 85 - DNA, 32 bp, Artificial Sequence
  Description of Artificial Sequence Synthetic oligonucleotide
cttgagatcctttttttctgcgcgtaatctgc

SEQ ID NO: 86 - DNA, 37 bp, Artificial Sequence
  Description of Artificial Sequence Synthetic oligonucleotide
tcagaagaactcgtcaagaaggcgatagaaggcgatg

SEQ ID NO: 87 - DNA, 48 bp, Artificial Sequence
  Description of Artificial Sequence Synthetic oligonucleotide
tggtttcttagacgtcaggtggcacttttcaaccggaattgccagctg

SEQ ID NO: 88 - DNA, 64 bp, Artificial Sequence
  Description of Artificial Sequence Synthetic oligonucleotide
ctaaactcgacctctacattttttgaaatttgctattttgttagagtcttttacaccatttgtc

SEQ ID NO: 89 - DNA, 39 bp, Artificial Sequence
  Description of Artificial Sequence Synthetic oligonucleotide
gccttctatcgccttctttgagctcatacacccaaacag

SEQ ID NO: 90 - DNA, 39 bp, Artificial Sequence
  Description of Artificial Sequence Synthetic oligonucleotide
agattacgcgcagaaaaaacaagaattcttactacgcac

SEQ ID NO: 91 - DNA, 71 bp, Artificial Sequence
  Description of Artificial Sequence Synthetic oligonucleotide
gtaaaagactctaacaaaatagcaaatttcgtcaaaaatgctaagaaatagactttagccttgagatgatg

SEQ ID NO: 92 - DNA, 22 bp, Artificial Sequence
  Description of Artificial Sequence Synthetic oligonucleotide
tcgggcaccgaactccccgaag

SEQ ID NO: 93 - DNA, 20 bp, Artificial Sequence
  Description of Artificial Sequence Synthetic oligonucleotide
gttagcgcttcggggagttc

SEQ ID NO: 94 - DNA, 19 bp, Artificial Sequence
  Description of Artificial Sequence Synthetic oligonucleotide
cggctgcacttgcacttgg

SEQ ID NO: 95 - DNA, 22 bp, Artificial Sequence
  Description of Artificial Sequence Synthetic oligonucleotide
tctgggattaaccaagtgcaag

SEQ ID NO: 96 - DNA, 21 bp, Artificial Sequence
  Description of Artificial Sequence Synthetic oligonucleotide
tgcgcatcgccttggaaagtg

SEQ ID NO: 97 - DNA, 20 bp, Artificial Sequence
  Description of Artificial Sequence Synthetic oligonucleotide
cgggcacgccactttccaag

SEQ ID NO: 98 - DNA, 21 bp, Artificial Sequence
  Description of Artificial Sequence Synthetic oligonucleotide
tagatcaccacattgagaaag

SEQ ID NO: 99 - DNA, 22 bp, Artificial Sequence
  Description of Artificial Sequence Synthetic oligonucleotide
tgtcggtagctttctcaatgtg

SEQ ID NO: 100 - DNA, 45 bp, Artificial Sequence
  Description of Artificial Sequence Synthetic oligonucleotide
ccgattggtatcacgcacgtcacgtgcgatcacatcggcatcgac

SEQ ID NO: 101 - DNA, 45 bp, Artificial Sequence
  Description of Artificial Sequence Synthetic oligonucleotide
atcgcacgtgacgtgcgtgataccaatcgggtcaaaaccgtagtg

SEQ ID NO: 102 - DNA, 23 bp, Artificial Sequence
  Description of Artificial Sequence Synthetic oligonucleotide
tggacgtcgatacgcacggctag

SEQ ID NO: 103 - DNA, 22 bp, Artificial Sequence
  Description of Artificial Sequence Synthetic oligonucleotide
caactcgctagccgtgcgtatc

SEQ ID NO: 104 - DNA, 22 bp, Artificial Sequence
  Description of Artificial Sequence Synthetic oligonucleotide
tggggttaaaaattggaaggag

SEQ ID NO: 105 - DNA, 26 bp, Artificial Sequence
  Description of Artificial Sequence Synthetic oligonucleotide
tttaaagtctccttccaatttttaac

SEQ ID NO: 106 - DNA, 21 bp, Artificial Sequence
  Description of Artificial Sequence Synthetic oligonucleotide
gccttaaccttgaccacactc

SEQ ID NO: 107 - DNA, 22 bp, Artificial Sequence
  Description of Artificial Sequence Synthetic oligonucleotide
gccggcagggagtgtggtcaag

SEQ ID NO: 108 - DNA, 21 bp, Artificial Sequence
  Description of Artificial Sequence Synthetic oligonucleotide
tcagacaacatgttcacgttg

SEQ ID NO: 109 - DNA, 22 bp, Artificial Sequence
  Description of Artificial Sequence Synthetic oligonucleotide
cggcggtgaaggcaacgtgaac

SEQ ID NO: 110 - DNA, 60 bp, Artificial Sequence
  Description of Artificial Sequence Synthetic oligonucleotide
ttgcatctaaactcgacctctacattttttatgtttatctttcctcaccgatatttcgtg

SEQ ID NO: 111 - DNA, 51 bp, Artificial Sequence
  Description of Artificial Sequence Synthetic oligonucleotide
cagattacgcgcagaaaaaaaggatctcaagactttagccttgagatgatg

SEQ ID NO: 112 - DNA, 53 bp, Artificial Sequence
  Description of Artificial Sequence Synthetic oligonucleotide
gccttctatcgccttcttgacgagttcttctgattcctcaccgatatttcgtg

SEQ ID NO: 113 - DNA, 56 bp, Artificial Sequence
  Description of Artificial Sequence Synthetic oligonucleotide
cagattacgcgcagaaaaaaaggatctcaagctaaattgtaagcgttaatattttg

SEQ ID NO: 114 - DNA, 20 bp, Artificial Sequence
  Description of Artificial Sequence Synthetic oligonucleotide
caggctaaaacgcgcacctg

SEQ ID NO: 115 - DNA, 20 bp, Artificial Sequence
  Description of Artificial Sequence Synthetic oligonucleotide
caggtgcgcgttttagcctg

SEQ ID NO: 116 - DNA, 20 bp, Artificial Sequence
  Description of Artificial Sequence Synthetic oligonucleotide
cagaccgtcaccacgatccg

SEQ ID NO: 117 - DNA, 20 bp, Artificial Sequence
  Description of Artificial Sequence Synthetic oligonucleotide
cggatcgtggtgacggtctg

SEQ ID NO: 118 - DNA, 21 bp, Artificial Sequence
  Description of Artificial Sequence Synthetic oligonucleotide
ctcgatacgatgcgggatatc

SEQ ID NO: 119 - DNA, 21 bp, Artificial Sequence
  Description of Artificial Sequence Synthetic oligonucleotide
gatatcccgcatcgtatcgag

SEQ ID NO: 120 - DNA, 20 bp, Artificial Sequence
  Description of Artificial Sequence Synthetic oligonucleotide
cacggttggtcagctcattc

SEQ ID NO: 121 - DNA, 20 bp, Artificial Sequence
  Description of Artificial Sequence Synthetic oligonucleotide
gaatgagctgaccaaccgtg

SEQ ID NO: 122 - DNA, 19 bp, Artificial Sequence
  Description of Artificial Sequence Synthetic oligonucleotide
cagcggacggaaatcctcc

SEQ ID NO: 123 - DNA, 19 bp, Artificial Sequence
  Description of Artificial Sequence Synthetic oligonucleotide
ggaggatttccgtccgctg

SEQ ID NO: 124 - DNA, 20 bp, Artificial Sequence
  Description of Artificial Sequence Synthetic oligonucleotide
ttacctccttaaagatcttc

SEQ ID NO: 125 - DNA, 20 bp, Artificial Sequence
  Description of Artificial Sequence Synthetic oligonucleotide
gaagatctttaaggaggtaa

SEQ ID NO: 126 - DNA, 19 bp, Artificial Sequence
  Description of Artificial Sequence Synthetic oligonucleotide
cgacggtttcgaaccaaac

SEQ ID NO: 127 - DNA, 19 bp, Artificial Sequence
  Description of Artificial Sequence Synthetic oligonucleotide
gtttggttcgaaaccgtcg

SEQ ID NO: 128 - DNA, 54 bp, Artificial Sequence
  Description of Artificial Sequence Synthetic oligonucleotide
gccttctatcgccttcttgacgagttcttctgattagcgcttggccgcgaaaac

SEQ ID NO: 129 - DNA, 24 bp, Artificial Sequence
  Description of Artificial Sequence Synthetic oligonucleotide
  modified_base (16)..(16): Phosphorothioate-A base
  modified_base (17)..(17): Phosphorothioate-A base
tcagaagaactcgtcaagaaggcg

SEQ ID NO: 130 - DNA, 24 bp, Artificial Sequence
  Description of Artificial Sequence Synthetic oligonucleotide
  modified_base (16)..(16): Phosphorothioate-T base
  modified_base (17)..(17): Phosphorothioate-T base
cttgagatcctttttttctgcgcg

SEQ ID NO: 131 - DNA, 24 bp, Artificial Sequence
  Description of Artificial Sequence Synthetic oligonucleotide
  modified_base (16)..(16): Phosphorothioate-A base
  modified_base (17)..(17): Phosphorothioate-A base
tcaagaaggcgatagaaggcgatg

SEQ ID NO: 132 - DNA, 28 bp, Artificial Sequence
  Description of Artificial Sequence Synthetic oligonucleotide
  modified_base (16)..(16): Phosphorothioate-A base
  modified_base (17)..(17): Phosphorothioate-T base
ttttttctgcgcgtaatctgctgcttgc

SEQ ID NO: 133 - DNA, 40 bp, Artificial Sequence
  Description of Artificial Sequence Synthetic oligonucleotide
  modified_base (16)..(16): Phosphorothioate-A base
  modified_base (17)..(17): Phosphorothioate-G base
tacgcgcagaaaaaaaggatctcaagactttagccttgag

SEQ ID NO: 134 - DNA, 29 bp, Artificial Sequence
  Description of Artificial Sequence Synthetic oligonucleotide
  modified_base (16)..(16): Phosphorothioate-C base
  modified_base (17)..(17): Phosphorothioate-C base
tcgggcaccgaactccccgaagcgctaac

SEQ ID NO: 135 - DNA, 28 bp, Artificial Sequence
  Description of Artificial Sequence Synthetic oligonucleotide
  modified_base (16)..(16): Phosphorothioate-G base
  modified_base (17)..(17): Phosphorothioate-G base
gagttcggtgcccgaggcgctgcttgag

SEQ ID NO: 136 - DNA, 29 bp, Artificial Sequence
  Description of Artificial Sequence Synthetic oligonucleotide
  modified_base (16)..(16): Phosphorothioate-T base
  modified_base (17)..(17): Phosphorothioate-T base
cggctgcacttgcacttggttaatcccag

SEQ ID NO: 137 - DNA, 34 bp, Artificial Sequence
  Description of Artificial Sequence Synthetic oligonucleotide
  modified_base (16)..(16): Phosphorothioate-A base
  modified_base (17)..(17): Phosphorothioate-A base
gtgcaagtgcagccgaaaatcggctttggctttg

SEQ ID NO: 138 - DNA, 30 bp, Artificial Sequence
  Description of Artificial Sequence Synthetic oligonucleotide
  modified_base (16)..(16): Phosphorothioate-A base
  modified_base (17)..(17): Phosphorothioate-A base
tgcgcatcgccttggaaagtggcgtgcccg

SEQ ID NO: 139 - DNA, 33 bp, Artificial Sequence
  Description of Artificial Sequence Synthetic oligonucleotide
  modified_base (16)..(16): Phosphorothioate-C base
  modified_base (17)..(17): Phosphorothioate-C base
ccaaggcgatgcgcaccgccagcgcccattttg

SEQ ID NO: 140 - DNA, 29 bp, Artificial Sequence
  Description of Artificial Sequence Synthetic oligonucleotide
  modified_base (16)..(16): Phosphorothioate-A base
  modified_base (17)..(17): Phosphorothioate-G base
tagatcaccacattgagaaagctaccgac

SEQ ID NO: 141 - DNA, 34 bp, Artificial Sequence
  Description of Artificial Sequence Synthetic oligonucleotide
  modified_base (16)..(16): Phosphorothioate-T base
  modified_base (17)..(17): Phosphorothioate-C base
caatgtggtgatctatcgcttacccaaaatcatg

SEQ ID NO: 142 - DNA, 33 bp, Artificial Sequence
  Description of Artificial Sequence Synthetic oligonucleotide
  modified_base (16)..(16): Phosphorothioate-C base
  modified_base (17)..(17): Phosphorothioate-A base
ccgattggtatcacgcacgtcacgtgcgatcac

SEQ ID NO: 143 - DNA, 30 bp, Artificial Sequence
  Description of Artificial Sequence Synthetic oligonucleotide
  modified_base (16)..(16): Phosphorothioate-G base
  modified_base (17)..(17): Phosphorothioate-T base
cgtgataccaatcgggtcaaaaccgtagtg

SEQ ID NO: 144 - DNA, 30 bp, Artificial Sequence
  Description of Artificial Sequence Synthetic oligonucleotide
  modified_base (16)..(16): Phosphorothioate-A base
  modified_base (17)..(17): Phosphorothioate-C base
tggacgtcgatacgcacggctagcgagttg

SEQ ID NO: 145 - DNA, 37 bp, Artificial Sequence
  Description of Artificial Sequence Synthetic oligonucleotide
  modified_base (16)..(16): Phosphorothioate-G base
  modified_base (17)..(17): Phosphorothioate-A base
gcgtatcgacgtccagactggatgttggtggatattg

SEQ ID NO: 146 - DNA, 31 bp, Artificial Sequence
  Description of Artificial Sequence Synthetic oligonucleotide
  modified_base (16)..(16): Phosphorothioate-G base
  modified_base (17)..(17): Phosphorothioate-A base
tggggttaaaaattggaaggagactttaaag

SEQ ID NO: 147 - DNA, 33 bp, Artificial Sequence
  Description of Artificial Sequence Synthetic oligonucleotide
  modified_base (16)..(16): Phosphorothioate-G base
  modified_base (17)..(17): Phosphorothioate-C base
caatttttaaccccagcctctaacccgcaagag

SEQ ID NO: 148 - DNA, 35 bp, Artificial Sequence
  Description of Artificial Sequence Synthetic oligonucleotide
  modified_base (16)..(16): Phosphorothioate-A base
  modified_base (17)..(17): Phosphorothioate-C base
gccttaaccttgaccacactccctgccggcgtttg

SEQ ID NO: 149 - DNA, 34 bp, Artificial Sequence
  Description of Artificial Sequence Synthetic oligonucleotide
  modified_base (16)..(16): Phosphorothioate-G base
  modified_base (17)..(17): Phosphorothioate-G base
ggtcaaggttaaggcgggtcaatatgtcggaatc

SEQ ID NO: 150 - DNA, 36 bp, Artificial Sequence
  Description of Artificial Sequence Synthetic oligonucleotide
  modified_base (16)..(16): Phosphorothioate-A base
  modified_base (17)..(17): Phosphorothioate-C base
tcagacaacatgttcacgttgccttcaccgccgatc

SEQ ID NO: 151 - DNA, 34 bp, Artificial Sequence
  Description of Artificial Sequence Synthetic oligonucleotide
  modified_base (16)..(16): Phosphorothioate-A base
  modified_base (17)..(17): Phosphorothioate-C base
gaacatgttgtctgaacgaggttcgatcagcatc

SEQ ID NO: 152 - DNA, 31 bp, Artificial Sequence
  Description of Artificial Sequence Synthetic oligonucleotide
  modified_base (16)..(16): Phosphorothioate-C base
  modified_base (17)..(17): Phosphorothioate-G base
ctatcgccttcttgacgagttcttctgattc

SEQ ID NO: 153 - DNA, 2848 bp, Artificial Sequence
  Description of Artificial Sequence Synthetic polynucleotide
aaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaagcttcaaccaaagcttcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcacggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttc

SEQ ID NO: 154 - DNA, 4255 bp, Artificial Sequence
  Description of Artificial Sequence Synthetic polynucleotide
tatttaagtattgtttgtgcacttgccctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaagcttcaaccaaagcttcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcacggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatt

SEQ ID NO: 155 - DNA, 2764 bp, Artificial Sequence
  Description of Artificial Sequence Synthetic polynucleotide
atgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaacaagaattcttactacgcaccaccctgccactcgtcgcaatactgttgcagttcattcagcatacgaccaacgtggaaaccatcgcaaaccgcgtggtgaacctggatcgccagcggcatcagaactttgtcaccctgggtgtagtatttacccatggtgaagaccggtgcgaaaaagttgtccatgttcgctacattcaggtcgaagctagtgaaggatacccaagggttcgcagatacgaagaacatattttcgatgaagccttttgggaaatacgcgagattctcaccgtaacacgcaacgtcctgagagtagatgtgcaggaactgacggaagtcgtcgtggtattcgctccacaggctagagaaggtttcggtctgttcgtgaaaaacggtgtagcacgggtgaacagagtcccagataaccagttcgccgtctttcatcgccatacgaaattccggatgggcgttcatgagacgcgccaggatgtggatgaaggccgggtagaatttgtgcttgtttttcttgacagtcttgaggaatgcggtgatgtcgagctgaacagtttggttgtaggtgcactgcgcaacggactggaacgcttcaaagtgctctttacgatgccactgagagatgtcaacggtcgtgtagccggtgatctttttttccattttagcttccttagctcctgaaaatctcgataactcaaaaaatacgccctcatactagatatctagatccggcccgatgcgtccggcgtagaggatctgaagatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttcagtgacacaggaacacttaacggctgacatgggaattctactgtttgggtgtatgagctcaaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttgaaaagtgccacctgacgtc

SEQ ID NO: 156 - DNA, 1604 bp, Artificial Sequence
  Description of Artificial Sequence Synthetic polynucleotide
tcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagagcttgatcccctgcgccatcagatccttggcggcgagaaagccatccagtttactttgcagggcttcccaaccttaccagagggcgccccagctggcaattccggttgaaaagtgccacctgacgtcatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaag

SEQ ID NO: 157 - DNA, 742 bp, Artificial Sequence
  Description of Artificial Sequence Synthetic polynucleotide
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaatggcaa

SEQ ID NO: 158 - DNA, 897 bp, Artificial Sequence
  Description of Artificial Sequence Synthetic polynucleotide
tgcagatgcagcttgaagcaaatgcagatacttcagtggaagaagaaagctttggcccacaacccatttcacggttagagcagtgtggcataaatgccaacgatgtgaagaaattggaagaagctggattccatactgtggaggctgttgcctatgcgccaaagaaggagctaataaatattaagggaattagtgaagccaaagctgataaaattctggctgaggcagctaaattagttccaatgggtttcaccactgcaactgaattccaccaaaggcggtcagagatcatacagattactactggctccaaagagcttgacaaactacttcaaggtggaattgagactggatctatcacagaaatgtttggagaattccgaactgggaagacccagatctgtcatacgctagctgtcacctgccagcttcccattgaccggggtggaggtgaaggaaaggccatgtacattgacactgagggtacctttaggccagaacggctgctggcagtggctgagaggtatggtctctctggcagtgatgtcctggataatgtagcatatgctcgagcgttcaacacagaccaccagacccagctcctttatcaagcatcagccatgatggtagaatctaggtatgcactgcttattgtagacagtgccaccgccctttacagaacagactactcgggtcgaggtgagctttcagccaggcagatgcacttggccaggtttctgcggatgcttctgcgactcgctgatgagtttggtgtagcagtggtaatcactaatcaggtggtagctcaagtggatggagcagcgatgtttgctgctgatcccaaaaaacctattggaggaaatatcatcgcccatgcatcaacaaccagattgtatc

SEQ ID NO: 159 - DNA, 908 bp, Artificial Sequence
  Description of Artificial Sequence Synthetic polynucleotide
tgaggaaaggaagaggggaaaccagaatctgcaaaatctacgactctccctgtcttcctgaagctgaagctatgttcgccattaatgcagatggagtgggagatgccaaagacggaagcggagctactaacttcagcctgctgaagcaggctggagacgtggaggagaaccctggacctatggctgctgttcctcaaaataatctacaggagcaactagaacgtcactcagccagaacacttaataataaattaagtctttcaaaaccaaaattttcaggtttcacttttaaaaagaaaacatcttcagataacaatgtatctgtaactaatgtgtcagtagcaaaaacacctgtattaagaaataaagatgttaatgttaccgaagacttttccttcagtgaacctctacccaacaccacaaatcagcaaagggtcaaggacttctttaaaaatgctccagcaggacaggaaacacagagaggtggatcaaaatcattattgccagatttcttgcagactccgaaggaagttgtatgcactacccaaaacacaccaactgtaaagaaatcccgggatactgctctcaagaaattagaatttagttcttcaccagattctttaagtaccatcaatgattgggatgatatggatgactttgatacttctgagacttcaaaatcatttgttacaccaccccaaagtcactttgtaagagtaagcactgctcagaaatcaaaaaagggtaagagaaacttttttaaagcacagctttatacaacaaacacagtaaagactgatttgcctccaccctcctctgaaagcgagcaaatagatttgactgaggaacagaaggatgactcagaatggttaagcagcgatgtgatttgcatcgatgatggccccatt

SEQ ID NO: 160 - DNA, 3520 bp, Artificial Sequence
  Description of Artificial Sequence Synthetic polynucleotide
gctgaagtgcatataaatgaagatgctcaggaaagtgactctctgaaaactcatttggaagatgaaagagataatagcgaaaagaagaagaatttggaagaagctgaattacattcaactgagaaagttccatgtattgaatttgatgatgatgattatgatacggattttgttccaccttctccagaagaaattatttctgcttcttcttcctcttcaaaatgccttagtacgttaaaggaccttgacacatctgacagaaaagaggatgttcttagcacatcaaaagatcttttgtcaaaacctgagaaaatgagtatgcaggagctgaatccagaaaccagcacagactgtgacgctagacagataagtttacagcagcagcttattcatgtgatggagcacatctgtaaattaattgatactattcctgatgataaactgaaacttttggattgtgggaacgaactgcttcagcagcggaacataagaaggaaacttctaacggaagtagattttaataaaagtgatgccagtcttcttggctcattgtggagatacaggcctgattcacttgatggccctatggagggtgattcctgccctacagggaattctatgaaggagttaaatttttcacaccttccctcaaattctgtttctcctggggactgtttactgactaccaccctaggaaagacaggattctctgccaccaggaagaatctttttgaaaggcctttattcaatacccatttacagaagtcctttgtaagtagcaactgggctgaaacaccaagactaggaaaaaaaaatgaaagctcttatttcccaggaaatgttctcacaagcactgctgtgaaagatcagaataaacatactgcttcaataaatgacttagaaagagaaacccaaccttcctatgatattgataattttgacatagatgactttgatgatgatgatgactgggaagacataatgcataatttagcagccagcaaatcttccacagctgcctatcaacccatcaaggaaggtcggccaattaaatcagtatcagaaagactttcctcagccaagacagactgtcttccagtgtcatctactgctcaaaatataaacttctcagagtcaattcagaattatactgacaagtcagcacaaaatttagcatccagaaatctgaaacatgagcgtttccaaagtcttagttttcctcatacaaaggaaatgatgaagatttttcataaaaaatttggcctgcataattttagaactaatcagctagaggcgatcaatgctgcactgcttggtgaagactgttttatcctgatgccgactggaggtggtaagagtttgtgttaccagctccctgcctgtgtttctcctggggtcactgttgtcatttctcccttgagatcacttatcgtagatcaagtccaaaagctgacttccttggatattccagctacatatctgacaggtgataagactgactcagaagctacaaatatttacctccagttatcaaaaaaagacccaatcataaaacttctatatgtcactccagaaaagatctgtgcaagtaacagactcatttctactctggagaatctctatgagaggaagctcttggcacgttttgttattgatgaagcacattgtgtcagtcagtggggacatgattttcgtcaagattacaaaagaatgaatatgcttcgccagaagtttccttctgttccggtgatggctcttacggccacagctaatcccagggtacagaaggacatcctgactcagctgaagattctcagacctcaggtgtttagcatgagctttaacagacataatctgaaatactatgtattaccgaaaaagcctaaaaaggtggcatttgattgcctagaatggatcagaaagcaccacccatatgattcagggataatttactgcctctccaggcgagaatgtgacaccatggctgacacgttacagagagatgggctcgctgctcttgcttaccatgctggcctcagtgattctgccagagatgaagtgcagcagaagtggattaatcaggatggctgtcaggttatctgtgctacaattgcatttggaatggggattgacaaaccggacgtgcgatttgtgattcatgcatctctccctaaatctgtggagggttactaccaagaatctggcagagctggaagagatggggaaatatctcactgcctgcttttctatacctatcatgatgtgaccagactgaaaagacttataatgatggaaaaagatggaaaccatcatacaagagaaactcacttcaataatttgtatagcatggtacattactgtgaaaatataacggaatgcaggagaatacagcttttggcctactttggtgaaaatggatttaatcctgatttttgtaagaaacacccagatgtttcttgtgataattgctgtaaaacaaaggattataaaacaagagatgtgactgacgatgtgaaaagtattgtaagatttgttcaagaacatagttcatcacaaggaatgagaaatataaaacatgtaggtccttctggaagatttactatgaatatgctggtcgacattttcttggggagtaagagtgcaaaaatccagtcaggtatatttggaaaaggatctgcttattcacgacacaatgccgaaagactttttaaaaagctgatacttgacaagattttggatgaagacttatatatcaatgccaatgaccaggcgatcgcttatgtgatgctcggaaataaagcccaaactgtactaaatggcaatttaaaggtagactttatggaaacagaaaattccagcagtgtgaaaaaacaaaaagcgttagtagcaaaagtgtctcagagggaagagatggttaaaaaatgtcttggagaacttacagaagtctgcaaatctctggggaaagtttttggtgtccattacttcaatatttttaataccgtcactctcaagaagcttgcagaatctttatcttctgatcctgaggttttgcttcaaattgatggtgttactgaagacaaactggaaaaatatggtgcggaagtgatttcagtattacagaaatactctgaatggacatcgccagctgaagacagttccccagggataagcctgtccagcagcagaggccccggaagaagtgccgctgaggagcttgacgaggaaatacccgtatcttcccactactttgcaagtaaaaccagaaatgaaaggaagaggaaaaagatgccagcctcccaaaggtctaagaggagaaaaactgcttccagtggttccaaggcaaagggggggtctgccacatgtagaaagatatcttccaaaacgaaatcctccagcatcattggatccagttcagcctcacatacttctcaagcgacatcaggagccaatagcaaattggggattatggctccaccgaagcctataaatagaccgtttcttaagccttcatatgcattct

SEQ ID NO: 161 - DNA, 1954 bp, Artificial Sequence
  Description of Artificial Sequence Synthetic polynucleotide
cataagggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatggggata

SEQ ID NO: 162 - DNA, 5082 bp, Artificial Sequence
  Description of Artificial Sequence Synthetic polynucleotide
cagggattgctacaatttatcaaagaagcttcagaacccatccatgtgaggaagtataaagggcaggtagtagctgtggatacatattgctggcttcacaaaggagctattgcttgtgctgaaaaactagccaaaggtgaacctactgataggtatgtaggattttgtatgaaatttgtaaatatgttactatctcatgggatcaagcctattctcgtatttgatggatgtactttaccttctaaaaaggaagtagagagatctagaagagaaagacgacaagccaatcttcttaagggaaagcaacttcttcgtgaggggaaagtctcggaagctcgagagtgtttcacccggtctatcaatatcacacatgccatggcccacaaagtaattaaagctgcccggtctcagggggtagattgcctcgtggctccctatgaagctgatgcgcagttggcctatcttaacaaagcgggaattgtgcaagccataattacagaggactcggatctcctagcttttggctgtaaaaaggtaattttaaagatggaccagtttggaaatggacttgaaattgatcaagctcggctaggaatgtgcagacagcttggggatgtattcacggaagagaagtttcgttacatgtgtattctttcaggttgtgactacctgtcatcactgcgtgggattggattagcaaaggcatgcaaagtcctaagactagccaataatccagatatagtaaaggttatcaagaaaattggacattatctcaagatgaatatcacggtaccagaggattacatcaacgggtttattcgggccaacaataccttcctctatcagctagtttttgatcccatcaaaaggaaacttattcctctgaacgcctatgaagatgatgttgatcctgaaacactaagctacgctgggcaatatgttgatgattccatagctcttcaaatagcacttggaaataaagatataaatacttttgaacagatcgatgactacaatccagacactgctatgcctgcccattcaagaagtcgtagttgggatgacaaaacatgtcaaaagtcagctaatgttagcagcatttggcataggaattactctcccagaccagagtcgggtactgtttcagatgccccacaattgaaggaaaatccaagtactgtgggagtggaacgagtgattagtactaaagggttaaatctcccaaggaaatcatccattgtgaaaagaccaagaagtgcagagctgtcagaagatgacctgttgagtcagtattctctttcatttacgaagaagaccaagaaaaatagctctgaaggcaataaatcattgagcttttctgaagtgtttgtgcctgacctggtaaatggacctactaacaaaaagagtgtaagcactccacctaggacgagaaataaatttgcaacatttttacaaaggaaaaatgaagaaagtggtgcagttgtggttccagggaccagaagcaggtttttttgcagttcagattctactgactgtgtatcaaacaaagtgagcatccagcctctggatgaaactgctgtcacagataaagagaacaatctgcatgaatcagagtatggagaccaagaaggcaagagactggttgacacagatgtagcacgtaattcaagtgatgacattccgaataatcatattccaggtgatcatattccagacaaggcaacagtgtttacagatgaagagtcctactcttttaagagcagcaaatttacaaggaccatttcaccacccactttgggaacactaagaagttgttttagttggtctggaggtcttggagatttttcaagaacgccgagcccctctccaagcacagcattgcagcagttccgaagaaagagcgattcccccacctctttgcctgagaataatatgtctgatgtgtcgcagttaaagagcgaggagtccagtgacgatgagtctcatcccttacgagaaggggcatgttcttcacagtcccaggaaagtggagaattctcactgcagagttcaaatgcatcaaagctttctcagtgctctagtaaggactctgattcagaggaatctgattgcaatattaagttacttgacagtcaaagtgaccagacctccaagctatgtttatctcatttctcaaaaaaagacacacctctaaggaacaaggttcctgggctatataagtccagttctgcagactctctttctacaaccaagatcaaacctctaggacctgccagagccagtgggctgagcaagaagccggcaagcatccagaagagaaagcatcataatgccgagaacaagccggggttacagatcaaactcaatggagctctggaaaaactttggatttaagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

(19) United States
(12) Patent Application Publication
(10) Pub. No.: US 2018/0245116 A1
(43) Pub. Date: Aug. 30, 2018

(54) COMPOSITIONS AND METHODS FOR NUCLEIC ACID ASSEMBLY

(71) Applicant: LIFE TECHNOLOGIES CORPORATION, Carlsbad, CA (US)

(72) Inventors: Xiquan LIANG, Escondido, CA (US); Chang-Ho BAEK, San Diego, CA (US); Lansha PENG, Poway, CA (US); Federico KATZEN, San Marcos, CA (US)

(21) Appl. No.: 15/873,477

(22) Filed: Jan. 17, 2018

Related U.S. Application Data
(62) Division of application No. 14/800,384, filed on Jul. 15, 2015, now abandoned.
(60) Provisional application No. 62/024,650, filed on Jul. 15, 2014.

(51) Int. Cl. C12P 19/34; C12N 15/66; C12N 15/64; C12N 15/10

(52) U.S. Cl. CPC C12P 19/34; C12N 15/66; C12N 15/64; C12N 15/1031; C12N 15/10

(57) ABSTRACT

The present disclosure generally relates to compositions and methods for the assembly of nucleic acid molecules into larger nucleic acid molecules. Also provided are compositions and methods for seamlessly connection of nucleic acid molecules with high sequence fidelity.

### CROSS-REFERENCE TO RELATED APPLICATIONS

The application is a divisional of U.S. application Ser. No. 14/800,384, filed Jul. 15, 2015, now pending, which claims the benefit of U.S. Provisional Application No. 62/024,650, filed Jul. 15, 2014, whose disclosure is incorporated by reference in its entirety.

### SEQUENCE LISTING

The instant application contains a Sequence Listing which has been submitted electronically in ASCII format and is hereby incorporated by reference in its entirety. Said ASCII copy, created on Jul. 13, 2015, is named LT00899_SL.txt and is 82,197 bytes in size.

### FIELD

The present disclosure generally relates to compositions and methods for the assembly of nucleic acid molecules into larger nucleic acid molecules. Provided are compositions and methods for seamless connection of nucleic acid molecules, in many instances, with high sequence fidelity.

### BACKGROUND

As genetic engineering has developed, a need for the generation of larger and larger nucleic acid molecules has also developed. In many instances, nucleic acid assembly methods involve the production of sub-assemblies (e.g., chemically synthesized oligonucleotides), followed by the generation of larger (e.g., annealing of oligonucleotides to form double-stranded nucleic acid molecules) and larger assemblies (e.g., ligation of double-stranded nucleic acid molecules).

The present disclosure generally relates to compositions and methods for efficient assembly of nucleic acid molecules.

### SUMMARY

The present disclosure relates, in part, to compositions and methods for efficient assembly of nucleic acid molecules. Three aspects of the invention, that may be used in combination or separately, are as follows:

1. The use of nuclease resistant regions near the termini (e.g., within 12, 15, 20, 30, 40, or 50 base pairs) of nucleic acid segments to limit digestion of these nucleic acid segments during the formation of single-stranded regions (e.g., single-stranded regions designed for hybridization to other nucleic acid segments).

2. The reconstitution of functional nucleic acid elements (e.g., selectable marker, origins or replication, etc.) for the purpose of selecting for correctly assembled nucleic acid molecules.

3. The stopping/inhibition of assembly reaction processes that can affect the stability of nucleic acid molecules prepared during the assembly process.

In some aspects, the invention relates to compositions and methods for covalently linking two nucleic acid segments, these method comprising: (a) incubating the two nucleic acid segments with one or more nuclease (e.g., exonuclease) under conditions that allow for digestion of termini of the two nucleic acid segments to form complementary single-stranded regions on each nucleic acid segment and hybridization of the complementary single-stranded regions, wherein each of the two nucleic acid segments comprises a nuclease resistant region within 30 nucleotides of the end of the complementary terminus, and (b) covalently connecting at least one strand of the hybridized termini formed in (a) resulting in the linkage of the two nucleic acid segments.

Steps (a) and (b), referred to above, may be performed in the same tube and/or at the same time. Further, the two or more nucleic acid segments may be simultaneously contacted with one or more nuclease (e.g., exonuclease) and one or more molecule with ligase activity (e.g., ligase, topoisomerase, etc.) in step (a). In such instances, the two or more nucleic acid segments may be contacted with the one or more nuclease first, followed by contacting with the one or more molecule ligase activity or the two or more nucleic acid segments with the one or more nuclease and the one or more molecule ligase activity at the same time.

The invention also includes compositions and methods in which three or more (e.g., four, five, eight, ten twelve, fifteen, etc.) nucleic acid segments are covalently linked to each other. Further, some of these nucleic acid segments may not contain a nuclease (e.g., exonuclease) resistant region, some may contain a single nuclease resistant region and some may contain two nuclease resistant regions. In most cases, nucleases resistant regions, when present will be within 30 base pairs of a terminus of the nucleic acid segment in which they are present.

In many instances, nucleic acid molecules prepared by methods of the invention will be replicable. Further, many of these replicable nucleic acid molecules will be circular (e.g., plasmids). Replicable nucleic acid molecules, regardless of whether they are circular, will generally be formed from the assembly of two or more (e.g., three, four, five, eight, ten, twelve, etc.) nucleic acid segments. In some instances, methods of the invention employ selection based upon the reconstitution of one or more (e.g., two, three, four, etc.) selection marker or one or more (e.g., two, three, four, etc.) origin of replication resulting from the linking of different nucleic acid segments. Further selection may result from the formation of a circular nucleic acid molecule, in instances where circularity is required for replication.

The invention also relates, in part, to compositions and methods for storing assembled nucleic acid molecules (e.g., nucleic acid molecules assembled by method disclosed herein). Stabilization of nucleic acid molecules is often facilitated by the inhibition of nucleic acid assembly activities (e.g., nuclease activities). Thus, the invention includes methods for the stabilization of nucleic acid molecules associated with the inhibition or elimination of activities (e.g., enzymatic activities) associated with the assembly process. One example is that methods of the invention include those involving the partial or full inactivated one or more enzyme contacting assembled nucleic acid molecules. This may be accomplished by the use of enzymatic inhibitors, pH changes, as well as other means.

In some instances, inhibition of enzymatic activity will be mediated by heating. While the temperatures required to inactivate enzymes differ with the particular enzyme or enzymes in the mixture, typically, heating will be to a temperature greater than 65° C. (e.g., 70° C., 75° C., 80° C., or 85° C.) for at least 10 minutes (e.g., 15 minutes, 20 minutes, 25 minutes, 30 minutes, etc.).

In many instances, after the partial or full inactivated one or more enzyme contacting assembled nucleic acid molecules, the assembled nucleic acid molecules will be stored at a temperature equal to or below 4° C. (e.g., −20° C., −30° C., −60° C., or −70° C). for at least 24 hours (e.g., 36 hours, two days, five days, seven days, two weeks, three weeks, one month, three months, six months, nine months, one year).

The invention also includes methods for assembling nucleic acid molecules, these methods comprising: (a) incubating a first nucleic acid segment with a nuclease (e.g., an exonuclease) under conditions that allow for partial digestion of at least one terminus of the first nucleic acid segment to form a single-stranded region, wherein the first nucleic acid segment contains a nuclease resistant region within 30 nucleotides of the at least one terminus, (b) preparing a reaction mixture containing the digested first nucleic acid segment formed in (a) with an undigested second nucleic acid segment under conditions that allow for the hybridization of termini with sequence complementarity, and (c) covalently connecting at least one strand of the hybridized termini formed in (b). The second nucleic acid segment of (b) may or may not contain a nuclease resistant region. In many instances, the at least one terminus of the second nucleic acid segment of (b) will contain a single-stranded region with sequence complementarity to the single-stranded region of the first nucleic acid molecules formed in step (a). Further, the nuclease of (a) may be an exonuclease and, more specifically, a 5′ to 3′ exonuclease or 3″ to 5′ exonuclease. Additionally, two or more nucleases are present in step (a). Further, the nuclease(s) present may retain partial or full functionality in step (b) or may be partially or fully inactivated.

The invention also includes methods for assembling nucleic acid molecules, these methods comprising: (a) incubating two or more nucleic acid segments with a nuclease (e.g., an exonuclease) under conditions that allow for partial digestion of at least one terminus of each of the two or more nucleic acid segments to generate single-stranded termini, wherein at least two of the two or more nucleic acid segments contain a nuclease resistant region within 30 nucleotides of at least one of their termini, (b) preparing a reaction mixture containing the digested nucleic acid segments prepared in (a) with one or more undigested nucleic acid segment under conditions that allow for the hybridization of termini with sequence complementarity, wherein at least one of the one or more undigested nucleic acid segment has region of sequence complementarity with at least one single-stranded terminus formed in (a), and (c) covalently connecting at least one strand of the hybridized termini formed in (b).

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram that shows some components of exemplary work flows related to the invention. Error correction may be performed at multiple steps within work flows.

FIG. 2 is a schematic showing two double stranded nucleic acid segments (NA1 and NA2), represented as A-B-C and C-B-D. Region B (a nuclease resistant region), as shown in the diagram, contains two phosphorothioate bonds. Region C in both nucleic acid segments, as shown in the diagram, is fifteen base pairs in length and share sequence complementarity with each other.

FIG. 3 shows variations of Region B (a nuclease resistant region). R represents a resistant base and S represents a sensitive base. Four variations of Region B are also shown with R and S bases on different strands and having a length of between two and four base pairs.

FIG. 4 shows the joining of two nucleic acid segments. One nucleic acid segment (NA1) has no nuclease resistant bases. The other nucleic acid segment (NA2) has a nuclease resistant region (Region B) that contains two phosphorothioate bonds. NA2, but not NA1, is treated with an exonuclease under conditions designed to generate a 15 base pair single-stranded region with sequence complementarity to one terminus of NA1. The result is that, with many of the connected nucleic acid segments, a “flap forms with one strand.

FIG. 5 is a schematic showing six double stranded nucleic acid segments. The two nucleic acid segments shown in black and grey each contain a marker (Marker 1 and Marker 2). The other four nucleic acid segments (numbered 1 through 4) have termini similar to those represented in FIG. 2. “X” designations mark regions of sequence homology.

FIG. 6 is a schematic showing the assembly of 10 DNA fragments for violacein synthesis genes (8-kb total insert size) using the positive-selection vector pYES8D in yeast. Panel A: Test complete assembly sets using three different types of insert fragments: pre-cloned, PCR-amplified, and synthetic DNA fragments. Panel B: Control assembly sets: missing one insert fragment (white downward arrows) at different positions, pYES8D with no insert, complete set but no positive selection, and pYES8 alone. Complement element 1 (CE1) for the TRP1-TR was added to the forward primer for the Vio-1 fragment. CE2 for the 2μ ori-TR was added to the reverse primer for the Vio-10 fragment. Results for colony number and cloning efficiency are summarized in the table at right panel. NA, not applicable.

FIG. 7 shows a schematic of an assembly of ten DNA fragments for V. cholerae pilABCD/pilMNOPQ region (9.9-kb total insert size) using the positive-selection vector pYES8D in yeast. An assembly set missing one insert fragment at pilMQ-1 position was tested as negative control (white downward arrow). Complement element 1 (CE1) for the TRP1-TR was added to the forward primer for the pilAD-1 fragment. CE2 for the 2μ ori-TR was added to the reverse primer for the piIMQ-5 fragment. Results for colony number and cloning efficiency are summarized in the table. NA, not applicable.

FIG. 8 shows a schematic of a gene assembly using the positive-selection vector pASE101 in E. coli using three reporter genes. In particular, the assembly of three reporter DNA fragments (2-kb total insert size) using the positive-selection vector pASE101L in E. coli is shown. An assembly set missing one insert fragment at lacZ-α position was tested as negative control (white downward arrow). Complement element 1 (CE1) for the truncated pUC ori (pUC ori-TR) was added to the forward primer for the gfp gene fragment. CE2 for the truncated Km^{R} (Km^{R}-TR) was added to the reverse primer for the cat gene fragment. Results for colony number and cloning efficiency are summarized in the table. NA, not applicable.

FIG. 9 shows the construction of a positive-selection vector pASE101 for nucleic acid assembly in E. coli. Panel A: PCR-amplified pUC-Km derivatives to identify complement elements (CE) for the truncated pUC ori (pUC ori-TR). Panel B: PCR•amplified pUC•Km derivative to identify CE for the truncated Km^{R}(Km^{R}•TR). Panel C: PCR-amplified positive-selection vector pASE101L. Panel D: Construction of pASE101 to propagate pASE101L in E. coli. PCR products were self-ligated and introduced into E. coli strain TOP10 or DH10B-T1 by transformation to test phenotype. Phenotypes of the constructs are summarized in the table. The forward/reverse primer set for each construct are shown as the numbered half arrows.

FIG. 10 is a flow chart of an exemplary process for synthesis of error-minimized nucleic acids.

FIG. 11 is a vector map of pYES8D.

FIG. 12 is a vector map of pYES8.

FIG. 13 shows an assembly of 10 DNA fragments for V. cholerae pilABCD/pilMNOPQ region (9.9-kb total insert size) using the positive-selection vector pYES8D in yeast. Two assembly sets, missing one insert fragment at pilMQ-1 position (white downward arrow) and no inserts, were tested as control experiments. The complementing sequences for the TRP1-TR (CE1) were added to the forward primer for the pilAD-1 fragment. The complementing sequences for the 2μ ori-TR (CE2) were added to the reverse primer for the pilMQ-5 fragment. Results for colony number and cloning efficiency are summarized in the table. NA, not applicable.

FIG. 14 is a vector map of pASE101.

FIG. 15 is a vector map of pASE_cont.

FIG. 16 shows ten fragment assembly into pASE101 and pASE_cont.

FIGS. 17A-17B show vector maps for pcDNA Rad51 BLM Exo1. The is vector contains 13,103 base pairs and was assembled from six fragments/segments using methods of the invention. The nucleotide sequence of this vector is set out in Table 14. Phosphorothioate bonds were located in the termini of the fragments along the lines of FIGS. 2-5.

### DETAILED DESCRIPTION

Definitions:

As used herein the term “sequence fidelity” refers to the level of sequence identity of a nucleic acid molecule as compared to a reference sequence. Full identity being 100% identical over the full length of the nucleic acid molecules being scored for sequence identity. Sequence fidelity can be measure in a number of ways, for example, by the comparison of the actual nucleotide sequence of a nucleic acid molecule to a desired nucleotide sequence (e.g., a nucleotide sequence that one wishes to be used to generate a nucleic acid molecule). Another way sequence fidelity can be measured is by comparison of sequences of two nucleic acid molecules in a reaction mixture. In many instances, the difference on a per base basis will be, on average, the same.

As used herein the term “exonuclease” refers to enzymes that cleaves nucleotides one from the end (exo) of a polynucleotide chain. Typically, their enzymatic mechanism involves hydrolyzing reactions that breaks phosphodiester bonds at either the 3′ or the 5′ end occurs. Exemplary exonucleases include Escherichia coli exonuclease I, Escherichia coli exonuclease III (3′ to 5′), Escherichia coli exonuclease VII, Escherichia coli exonuclease VIII, bacteriophage lambda exonuclease (5′ to 3′), exonuclease T (3′ to 5′), bacteriophage T5 Exonuclease, and bacteriophage T7 exonuclease (5′ to 3′).

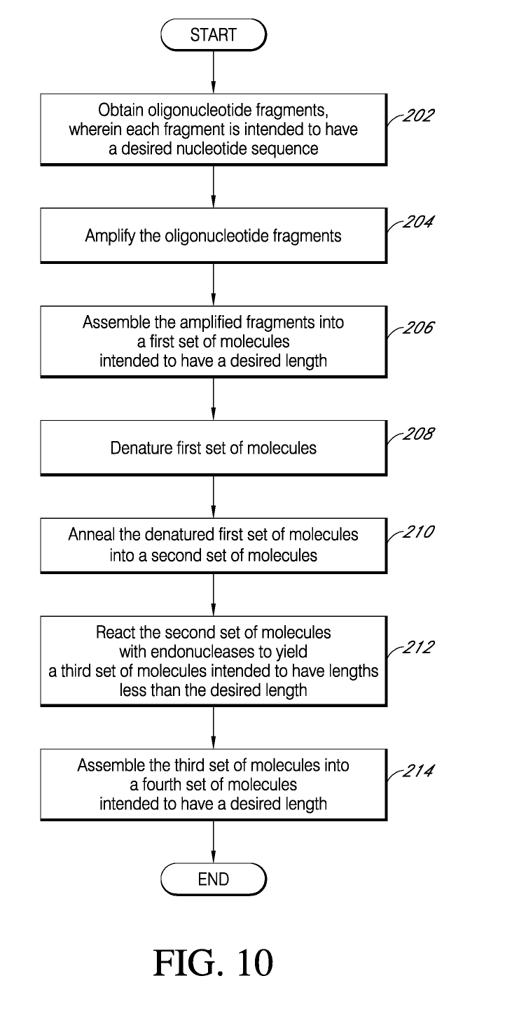

As used herein the term “error correction” refers to changes is the nucleotide sequence of a nucleic acid molecule to alter a defect. These defects can be mis-matches, insertions, and/or substitutions. Defects can occur when a nucleic acid molecule that is being generated (e.g., by chemical or enzymatic synthesis) is intended to contain a particular base at a location but a different base is present at that location. One error correction workflow is set out in FIG. 10.

As used herein the term “selectable marker” refers to a nucleic acid segment that allows one to select for or against a nucleic acid molecule or a cell that contains it, often under particular conditions. Examples of selectable markers include but are not limited to: (1) nucleic acid segments that encode products which provide resistance against otherwise toxic compounds (e.g., antibiotics); (2) nucleic acid segments that encode products which are otherwise lacking in the recipient cell (e.g., tRNA genes, auxotrophic markers); (3) nucleic acid segments that encode products which suppress the activity of a gene product; (4) nucleic acid segments that encode products which can be readily identified (e.g., phenotypic markers such as (P-galactosidase, green fluorescent protein (GFP), yellow flourescent protein (YFP), red fluorescent protein (RFP), cyan fluorescent protein (CFP), and cell surface proteins); (5) nucleic acid segments that bind products which are otherwise detrimental to cell survival and/or function; (6) nucleic acid segments that bind products that modify a substrate (e.g., restriction endonucleases); (7) nucleic acid segments that can be used to isolate or identify a desired molecule (e.g., specific protein binding sites); (8) nucleic acid segments, which when absent, directly or indirectly confer resistance or sensitivity to particular compounds; and/or (9) nucleic acid segments that encode products which either are toxic (e.g., Diphtheria toxin) or convert a relatively non-toxic compound to a toxic compound (e.g., Herpes simplex thymidine kinase, cytosine deaminase) in recipient cells.

A “counter selectable” marker (also referred to herein a “negative selectable marker”) or marker gene as used herein refers to any gene or functional variant thereof that allows for selection of wanted vectors, clones, cells or organisms by eliminating unwanted elements. These markers are often toxic or otherwise inhibitory to replication under certain conditions which often involve exposure to a specific substrates or shift in growth conditions. Counter selectable marker genes are often incorporated into genetic modification schemes in order to select for rare recombination or cloning events that require the removal of the marker or to selectively eliminate plasmids or cells from a given population. One example of a negative selectable marker system widely used in bacterial cloning methods is the CcdA/CCdB Type II Toxin-antitoxin system.

Overview:

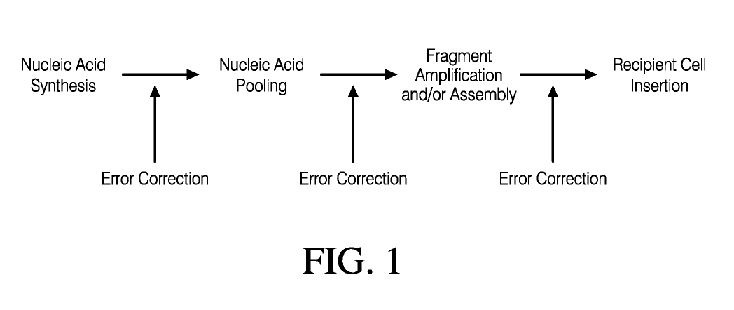

The invention relates, in part, to compositions and methods for the preparation of nucleic acid molecules. While the invention has numerous aspects and variations associated with it, some of these aspects and variations of applicability of the technology may be represented with the exemplary work flow shown in FIG. 1.

FIG. 1 shows a work flow including nucleic acid synthesis (e.g., chemical or enzymatic synthesis), pooling of synthesized nucleic acid molecules, amplification of pooled nucleic acid molecules, assembly of nucleic acid molecules (amplified and non-amplified nucleic acid molecules), and insertion of assembled nucleic acid molecules into recipient cells. Further indicated are locations in the work flow where error correction may be employed. As one skilled in the art would understand, error correction, when performed, may be employed at one or more locations in the work flow and multiple rounds of error correction may be employed at each point in the work flow where it is performed.

Multiple variations of the work flow represented in FIG. 1 may be used. For example, in instances where nucleic acid molecules are generated for in vitro transcription, recipient cell insertion may be omitted. As another example, sequencing of pre-assembly components of and/or assembled nucleic acid molecules may be used instead of or in additional to error correction of assembled nucleic acid molecules. Further, nucleic acid molecules determined to have the desired nucleotide sequence may be selected for, for example, insertion into recipient cells.

In one aspect, methods are provided for the production of nucleic acid molecules having high “sequence fidelity”. This high sequence fidelity can be achieved by, for example, one two or all three of the following: accurate nucleic acid synthesis, error correction, and sequence verification.

Described herein are a number of technologies with applicability to work flows such as those shown in FIG. 1, as well as other work flows. In one aspect, the invention is directed to method for the generation for nucleic acid molecules with high sequence fidelity as compared to nucleic acid molecules which are sought.

Nucleic Acid Assembly:

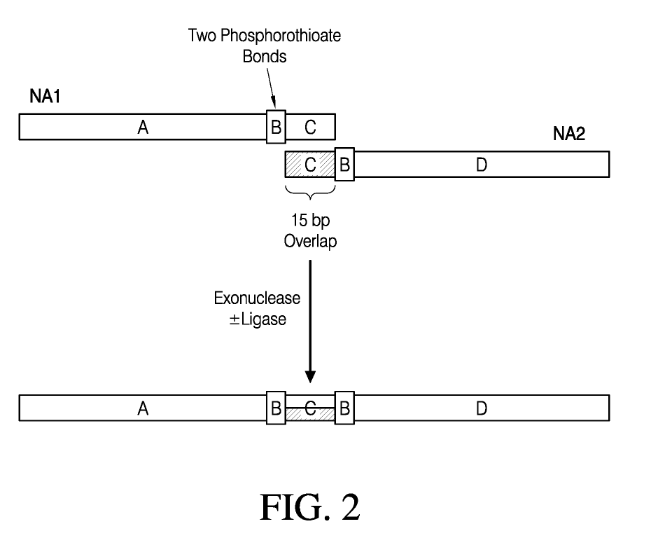

One exemplary embodiment of assembly technology described herein is set out in FIG. 2. FIG. 2 schematically shows exemplary assembly methods through which two nucleic acid segments (NA1 and NA2) are connected. In this exemplification, each nucleic acid segment has a region of sequence complementarity (Region C) and a region containing two phosphorothioate bonds (Region B) on the same strand or different strands but typically on different strands (e.g., within from about 4 to about 40 nucleotides of either from 3′ or 5′ terminus). When exposed to an exonuclease (e.g., a 5′ or 3′ exonuclease), one strand of Region C is “chewed back” up to Region B, generating termini capable of hybridizing with each other under suitable conditions (e.g., temperature, pH, ionic strength, etc.). Upon hybridization, a ligase (or a ligase activity) seals the nicks in each strand resulting in each strand resulting in the formation of a ligated nucleic acid molecule containing no nicks.

Nucleic acid segments such as those used in the work flow of FIG. 2 will typically contain a chemical modification that renders termini of nucleic acid strands resistant to nuclease activity (e.g., endonuclease digestion). One example of such a chemical modification, phosphorothioate bonds, is shown in FIG. 2. Other chemical modifications include methylphosphonates, 2′ methoxy ribonucleotides, locked nucleotides (LNAs), and 3′ terminal phosphoroamidates.

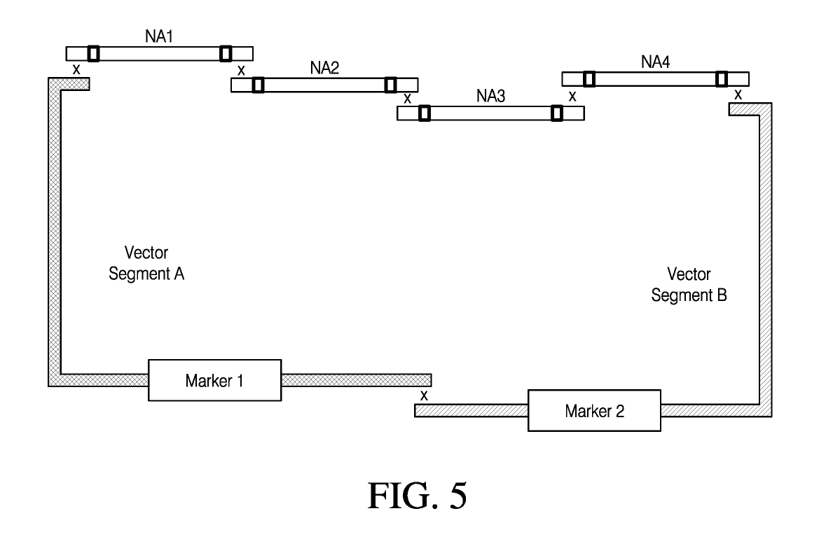

Only one terminus of each nucleic acid segment represented in FIG. 2 is shown as containing chemical modifications. In many instances, both termini will be chemically modified (similar to as shown for nucleic acid segments 1 through 4 in FIG. 5).

Numerous parameters may be designed and chosen to assemble, for example, different numbers of segments and nucleic acid segments of different length. Parameters may also be altered that result in increased efficiency of nucleic acid assembly for particular applications.

Using the schematic representation of FIG. 2 for reference, physical parameters such as the total lengths of NA1 and/or NA2, the lengths of Regions A and/or D, the lengths of one or both Region C, and the number of bases in one or both Region B may be varied. One chemical parameter that may be varied is the type of types of nuclease resistant bases incorporated into Region B. Other parameters that may be altered are the concentration of nucleic acid segments for assembly, the units of activity of enzymes (e.g., exonuclease, ligase, etc.) in the reactions mixture(s), pH, salt concentration, temperature, etc.

With respect to lengths of Regions A and/or D, when a nucleic acid molecule is longer than a certain length, the termini act as though they are, for purposes of association with other nucleic acid molecules, in effect different molecules. This, and other factors associated with long nucleic acid molecules (e.g., fragility), means that nucleic acid segment length is one factor for optimization with respect to assembly efficiency.

In some aspects of the invention, nucleic acid segment length will vary from about 20 base pairs to about 5,000 base pairs, from about 100 base pairs to about 5,000 base pairs, from about 150 base pairs to about 5,000 base pairs, from about 200 base pairs to about 5,000 base pairs, from about 250 base pairs to about 5,000 base pairs, from about 300 base pairs to about 5,000 base pairs, from about 350 base pairs to about 5,000 base pairs, from about 400 base pairs to about 5,000 base pairs, from about 500 base pairs to about 5,000 base pairs, from about 700 base pairs to about 5,000 base pairs, from about 800 base pairs to about 5,000 base pairs, from about 1,000 base pairs to about 5,000 base pairs, from about 100 base pairs to about 4,000 base pairs, from about 150 base pairs to about 4,000 base pairs, from about 200 base pairs to about 4,000 base pairs, from about 300 base pairs to about 4,000 base pairs, from about 500 base pairs to about 4,000 base pairs, from about 50 base pairs to about 3,000 base pairs, from about 100 base pairs to about 3,000 base pairs, from about 200 base pairs to about 3,000 base pairs, from about 250 base pairs to about 3,000 base pairs, from about 300 base pairs to about 3,000 base pairs, from about 400 base pairs to about 3,000 base pairs, from about 600 base pairs to about 3,000 base pairs, from about 800 base pairs to about 3,000 base pairs, from about 100 base pairs to about 2,000 base pairs, from about 200 base pairs to about 2,000 base pairs, from about 300 base pairs to about 1,500 base pairs, etc.

Nucleic acid segments used for assembly may be derived from a number of sources, for example, they may be cloned, derived from polymerase chain reactions, or chemically synthesized. Chemically synthesized nucleic acids tend to be of less than 100 nucleotides in length. PCR and cloning can be used to generate much longer nucleic acids. Further, the percentage of erroneous bases present in nucleic acids (e.g., nucleic acid segment) is, to some extent, tied to the method by which it is made. Typically, chemically synthesized nucleic acids have the highest error rate.

The length of the “hybridization” region, Region C, may also vary. The lengths of Region C may vary on each nucleic acid segment. FIG. 2 shows Region C being 15 base pairs in length on each nucleic acid segment. The lengths of Region C on each nucleic acid segment may vary with factor such as AT/CG content (due to A:T having two hydrogen bonds and C:G having three hydrogen bonds), the number of nucleic acid segments being assembled, the lengths of the nucleic acid segments, and the incubation conditions (e.g., salt concentration, pH, temperature, etc.).

Typically, Region C will be, independently, on one or both segments in ranges of from about 1 to about 100 base pairs, from about 2 to about 100 base pairs, from about 10 to about 100 base pairs, from about 15 to about 100 base pairs, from about 20 to about 100 base pairs, from about 5 to about 80 base pairs, from about 10 to about 80 base pairs, from about 20 to about 80 base pairs, from about 30 to about 80 base pairs, from about 40 to about 80 base pairs, from about 25 to about 65 base pairs, from about 35 to about 65 base pairs, from about 1 to about 50 base pairs, from about 2 to about 50 base pairs, from about 3 to about 50 base pairs, from about 5 to about 50 base pairs, from about 6 to about 50 base pairs, from about 7 to about 50 base pairs, from about 8 to about 50 base pairs, from about 10 to about 50 base pairs, from about 12 to about 50 base pairs, from about 13 to about 50 base pairs, from about 14 to about 50 base pairs, from about 15 to about 50 base pairs, from about 18 to about 50 base pairs, from about 20 to about 50 base pairs, from about 1 to about 35 base pairs, from about 5 to about 30 base pairs, from about 5 to about 25 base pairs, from about 5 to about 20 base pairs, from about 5 to about 18 base pairs, from about 8 to about 50 base pairs, from about 8 to about 35 base pairs, from about 8 to about 30 base pairs, from about 8 to about 25 base pairs, from about 8 to about 20 base pairs, from about 10 to about 40 base pairs, from about 10 to about 35 base pairs, from about 10 to about 30 base pairs, from about 10 to about 25 base pairs, from about 10 to about 20 base pairs, etc.

The invention includes compositions and methods for nucleic acid assembly where the length or Region C varies with the sequence of this region. In particular, the invention includes reaction mixtures where nucleic acid segments with higher amount of As and Ts in Region C have a longer Region C than nucleic acid segments with a higher amount of Cs and Gs. As an example, Region C of a nucleic acid segment with 60% C and G and 40% A and T may be 12 base pairs in length. Region C of a nucleic acid segment with 60% A and T and 40% C and G may be 18 base pairs in length. Further, both of these nucleic acid segments may be assembled in the same reaction mixture.

Table 1 shows an exemplary relationship between the A/T:C/G content and length of Region C. Region C may also be of different lengths when present at both termini of a nucleic acid segment.

TABLE 1
Exemplary Region C (Hybridization Region)
Lengths and A/T:C/G Content

| Number of A/T Base | | Length of Region C | % Δ |
|---|---|---|---|
| Pairs | % A/T | (Base Pairs) | in Length |
| 5 | 33.3% | 9 | 40% |
| 6 | 40% | 12 | 20% |
| 7 or 8 | 46.7%/53.3% | 15 | NA |
| 9 | 60% | 18 | 20% |
| 10 | 66.7% | 21 | 40% |

The invention thus includes methods for assembling two or more nucleic acid segments, wherein one nucleic acid segment comprises at least one terminus with sequence homology to a second nucleic acid segment (e.g., Region C), wherein the region of homology varies in length as a function of the A/T:C/G ratio, with longer regions of sequence homology being present where the termini have higher A/T:C/G ratios. In some instances, one or both nucleic acid segment with sequence homology at their termini will contain an exonuclease resistant region (e.g., Region B).

In many instances, Regions C will be designed such that the two regions share 100% sequence complementarity after nuclease digestion. In some instances, sequence complementarity will be below 100% (e.g., greater than 75%, greater than 80%, greater than 85%, greater than 90%, greater than 95%, between 75% and 99%, between 75% and 95%, between 75% and 90%, between 75% and 85%, between 80% and 99%, between 80% and 95%, between 85% and 99%, between 85% and 95%, etc.).

Further, incubation conditions may be adjusted such that there is, on average, partial or complete nuclease digestion of one strand of Region C. Also, conditions may be adjusted such that either the 3′ strand or the 5′ strand is digested. This may be determined by the choice of nuclease used (e.g., exonuclease). In particular, one or more 3′-exonuclease or 5′ exonuclease may be used. For example, two or more exonucleases may be used to digest termini of nucleic acid segments.

The length, number and spacing of nuclease resistant bases in Region B may also vary. In some instances, Region B will be bounded by nuclease resistant bases. In other instances, Region B will contain non-resistant bases abutting Region C. This may be useful instances where one seeks to add one or more bases (e.g., restriction sites) to final assembly products that may or may not be translated. With reference to FIG. 2, the junction between

Regions B and C will generally be determined by the overlap region (Region C) between nucleic acid 1 (NA1) and nucleic acid 2 (NA2).

Nuclease resistant bases will normally be in only one strand of nucleic acid segments to be joined but may be present in both strands.

The length of Region B may be as short as one base pair or substantially longer than one base pair. In some instances, the length of Region B will be from about one to about twenty base pairs, from about one to about fifteen base pairs, from about one to about ten base pairs, from about one to about six base pairs, from about one to about four base pairs, from about one to about two base pairs, from about two to about twenty base pairs, from about two to about ten base pairs, from about two to about five base pairs, from about three to about twenty base pairs, from about three to about ten base pairs, from about three to about five base pairs, etc.

The number of nuclease resistant bases in Region B may also vary. For example, the number of bases may be from about one to about ten, from about two to about ten, from about three to about ten, from about four to about ten, from about five to about ten, from about two to about five, from about two to about four, etc.

Other parameters that may be varied include the concentration of nucleic acid segments present and the ratio of these segments. In many instances, the nucleic acid segment concentration will be adjusted in combination with the concentration of nuclease and enzyme with ligase activity. Further, the ratio of nucleic acid segments to each other will often be essentially 1:1 but ratios may vary for particular applications. For example, when hybridization termini are AT rich (e.g., greater than 50%, 55%, 60%, 65% AT), these nucleic acid segments may be present in a higher ratio than nucleic acid segments with non-AT rich hybridization termini.

Nucleic acid segments such as those represented in FIG. 2 may be generated by polymerase chain reaction (PCR) using primers containing nuclease resistant modifications. Such nucleic acid segments may also be generated by other methods such as chemical synthesis.

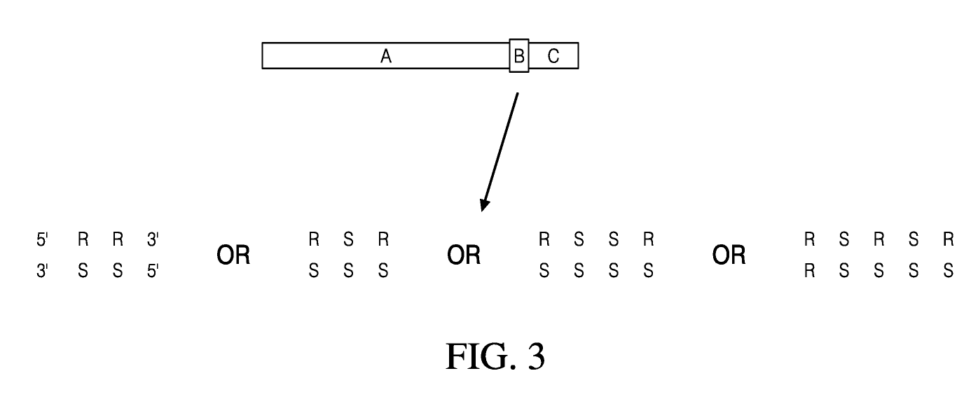

FIG. 3 shows some exemplary spacing of nuclease resistant bases in Region B. The far left shows two nuclease resistant bases (R) in one strand and two nuclease sensitive bases (S) in the other strand. The far left shows a Region B that is five base pairs in lengths with interspersed nuclease resistant bases in one strand. A single nuclease resistant base in the other strand and this base is located in Region B abutting Region A. One advantage of having a nuclease resistant base at this location is that provides nuclease resistance for the inhibition of digestion of Region B into Region A by exonucleases.

In some embodiments, two or more nucleic acid segments may be digested with exonucleases together or separately, then combined for assembly. In such instances, the same or different exonuclease may be used to digest termini or each fragment. Similarly, digestion reaction conditions may be the same or different the nucleic acid segments.

If desired, amplification of these nucleic acid molecules (e.g., polymerase chain reaction) may also be employed to generate nucleic acid molecules without phosphorothioate bonds.

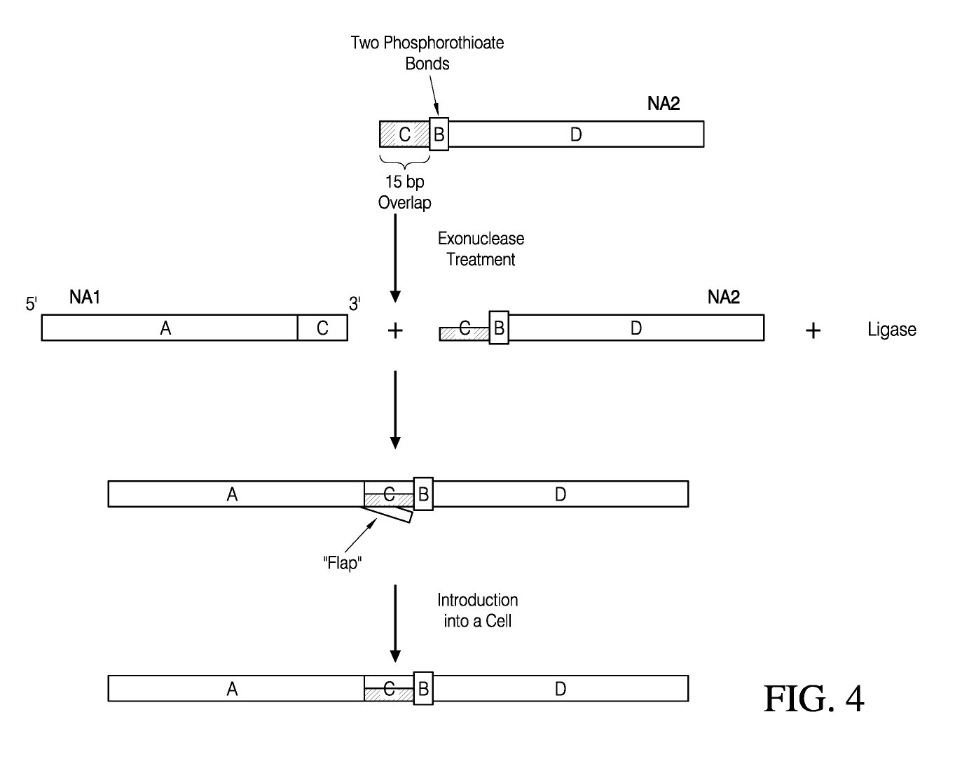

FIG. 4 shows a variation of the process shown in FIG. 2, where only one of the two nucleic acid segments to be joined at their termini is susceptible to nuclease action. In such instances, a blunt end may be joined to a “sticky” end through “strand invasion”. Strand invasion results in the formation of a “flap”, which is a single stranded region that protrudes from the connected nucleic acid segments. Strand invasion mechanisms are set out in U.S. Pat. No. 7,528, 241, the entire disclosure of which is incorporated herein by reference. A ligase or a molecule with ligase activity (e.g., a topoisomerase) may be used to connect the strand the recessed strand of the “invading” terminus to the 3′ strand of the blunt terminus of NA1.

In many instances of embodiments shown in FIG. 2, elimination of the “flap” will be performed after introduction into a cell by cellular nucleic acid repair mechanisms. Also, ligation of both strands may also occur intracellularly. In such instances, the two nucleic acid segments would not be covalently bound to each other until after introduction into a cell.

FIG. 5 is a schematic showing the assembly of six nucleic acid segments using methods of the invention. In this representation, two vector segments (Vector Segment A and Vector Segment B) are joined to four nucleic acid segments numbered 1 through 4. While FIG. 5 is directed to the assembly of a closed, circular vector, assemblies may be linear nucleic acid molecules. Compositions and methods are also provide for the preparation of linear nucleic acid molecules (e.g., linear vectors, sub-components of a larger nucleic acid molecule, nucleic acid molecules suitable for homologous recombination, etc.).

When a replicable, circular vector is generated, two types of selection are employed in the workflow of FIG. 5. One selection is based upon the formation of a circular nucleic acid molecule. An origin of replication, for example, may be used that allows for replication of a nucleic acid molecule when that molecule is circular. Thus, vector replication only occurs when circular nucleic acid molecules are formed. The assembly scheme in FIG. 5 is designed to result in the assembly of a circular nucleic acid molecule only when suitable termini are joined, resulting in the formation of a nucleic acid molecule containing Vector Segment A, Vector Segment B and nucleic acid segments 1 through 4. Of course, other combinations of the six nucleic acid segments can form from spurious connections between nucleic acid segments. In such cases, replicable nucleic acid molecules may be screened by methods such as gel electrophoresis and nucleotide sequencing to identify correct assemblies.

A second type of selection involves the use of selectable markers. Vector Segment A and Vector Segment B shown in FIG. 5 each contain a selectable marker. Any number of selectable markers and/or vector segments may be used. If two selective agents are used (e.g., ampicillin and puromycin), then only nucleic acid molecules containing both selectable markers (e.g., ampicillin resistance and puromycin resistance) will confer a resistant phenotype on cells. Thus, compositions and method of the invention include the presence and use of multiple selectable markers and resistance cassettes. These selectable markers may be present in assembled constructs produced using methods of the invention.

The invention further includes methods involving multiple selection methods for obtaining assembled nucleic acid molecules containing desired nucleic acid segments. In one embodiment, the invention includes methods for selecting assembled nucleic acid molecules through a combination of the generation of replicable vectors (e.g., recircularized vectors) and one or more selectable marker.

In some instances, vector segments may be distinguished from other nucleic acid segments in that they contain components in that they will generally contain components (e.g., functional components) normally found on. Examples of such components include origins or replication, long terminal repeats, selectable markers, promoters and antidote coding sequences (e.g., ccdA coding sequences for counter-acting toxic effects of ccdB). However, all nucleic acid segments assembled by methods described herein may contain such components. For example, when nucleic acid segments are assembled to form an operon, the assembled nucleic acid segments will often contain promoter and terminator sequences. Further, in some instances when a vector is assembled, the only segments that will be assembled will be vector segments.

The invention thus includes methods for the assembly of nucleic acid segments where some of the nucleic acid segments contain selectable markers or have functionalities that are otherwise required for replication (e.g., contain an origin of replication). As noted above, the number of nucleic acid segments assembled by methods of the invention may vary greatly. For example, the number of nucleic acid fragments/segments that may be assembled by methods of the invention include from about two to about fifty, from about three to about fifty, from about four to about fifty, from about two to about five, from about two to about ten, from about two to about fifteen, from about two to about twenty, from about three to about five, from about three to about ten, from about three to about twenty, from about four to about six, from about four to about ten, from about four to about fifteen, from about four to about twenty, from about five to about ten, from about five to about twenty, from about five to about thirty, from about five to about forty, from about eight to about fifteen, etc.

Further, the number of nucleic acid segments that do not contain components that confer selective or other replication related functionality may also vary. In general, the number of “non-selective” assembly components will be greater than the number of “selective” assembly components and the ratio of these two components may vary from about 2:1 to about 1:1, from about 2:1 to about 1.1:1, from about 3:1 to about 1.1:1, from about 5:1 to about 1.1:1, from about 6:1 to about 1.1:1, from about 7:1 to about 1.1:1, from about 8:1 to about 1.1:1, from about 10:1 to about 1.1:1, from about 15:1 to about 1.1:1, from about 20:1 to about 1.1:1, from about 10:1 to about 2:1, from about 10:1 to about 3:1, from about 10:1 to about 4:1, from about 10:1 to about 5:1, from about 10:1 to about 6:1, etc.

In the representation of FIG. 5, the two vector segments contain no nuclease resistant bases at either of their termini and nucleic acid segments 1 through 4 contain nuclease resistant bases at both of their termini. As one skilled in the art would understand, some termini may contain nuclease resistant bases and some may not. Some factors that will determine will often determine which termini contain nuclease resistant bases include ease of or difficulty in making of chemically modified nucleic acid molecules, assembly efficiency of the particular system, and “downstream” work-flow issues associated with the inclusion of modified bases in products nucleic acid molecules.

The six nucleic acid segments represented in FIG. 5 may be exposed to one or more nuclease prior to contact with each other or while in contact with each other. Further, groups of the nucleic acid segments may be exposed to one or more nuclease or some of the nucleic acid segments may be exposed to one or more nuclease followed by incubation of undigested nucleic acid segments and nuclease digested nucleic acid segments in the presence of one or more nuclease. Thus, the invention includes, for example, workflows in which nucleic acid segments containing one or more nuclease resistant bases near one or both termini are contacted with one or more nuclease, then contacted with undigested nucleic acid segments. The undigested nucleic acid segments may have 5′ or 3′ overhangs at one or both termini or may be blunt ended at both termini.

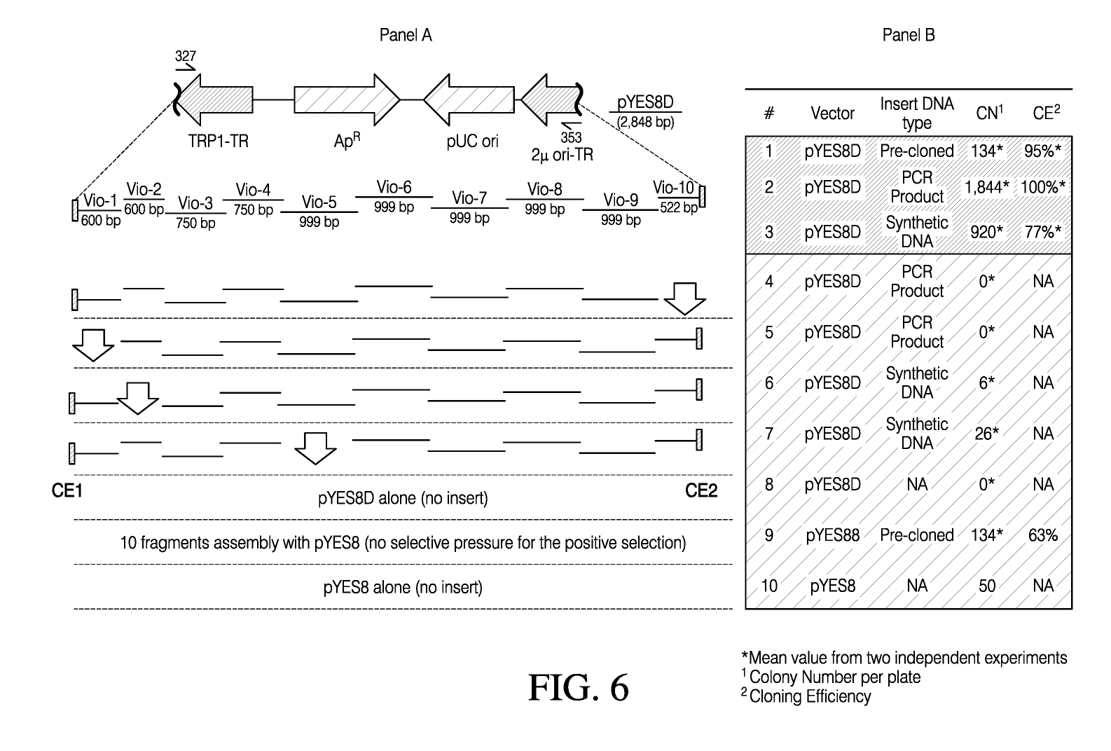

FIG. 6 shows a nucleic acid assembly scheme for the assembly of ten nucleic acid segments and a pYES8D vector segment. In this experiment eleven fragments with a total size of almost 13,000 base pairs were assembled. Correct assembly of these eleven nucleic acid segments results in the reconstitution of two vector components: TRP1 (yeast tryptophan auxotrophic marker) and the 2μ origin of replication. Also present on the vector back bone shown in FIG. 6 are a full length pUC origin of replication and a full length ampicillin resistance marker.

The right hand side of FIG. 6 shows data associated with assembly experiments. As can be seen the highest cloning efficiency was seen with PCR amplified nucleic acid segments. PCR generated and pre-cloned nucleic acids tend to be of higher purity than chemically synthesized ones. This may be one reason for the high cloning efficiency seen PCR amplified nucleic acid segments. FIG. 6A shows the experimental assembly and FIG. 6B shows a series of control assemblies. Assemblies missing one fragment at different positions (white blank arrow) gave zero or very low colony output indicating that every single fragment is important for successful assembly. Assembly of the truncated vector pYES8D with no insert showed no colony output, whereas assembly of the pYES8 (no positive selection) vector alone gave some false-positive background colonies. Thus this zero background from pYES8D-based DNA assembly may contribute to the higher cloning efficiency (95%) for pre-cloned DNA than pYES8-based assembly (63%).

Correctly assembled nucleic acid molecules resulting from the work flow shown in FIG. 6 are capable of replicating in both Escherichia coli and Saccharomyces cerevisiae. Thus, initial cloning may be done in E. coli in the presence of ampicillin, followed by transfer to S. cerevisiae for selection of full length, correctly assembled vectors. Alternatively, initial cloning may be done in S. cerevisiae for selection of full length, correctly assembled vectors, followed by transfer to E. coli if desired.

The invention thus provides compositions and methods for the preparation of shuttle vectors. These shuttle vectors may be screened for full length, correctly assembly in one organism (e.g., a eukaryotic cell), followed by transfer to another organism (e.g., a prokaryotic cell).

The invention also provides compositions and methods for the assembly of nucleic acid segments involving the reconstitution of one or more selectable markers and/or one or more origin of replication. In many instances, two functional components required for cell survival will be reconstituted in methods of the invention.

Compositions and methods of the invention are also useful for the preparation of nucleic acid molecules that encode counter-selectable markers (e.g., ccdB). Such vectors may be generated in a number of different ways. Vectors with counter-selectable markers may be generated by introducing assembled nucleic acid molecules into a cell that is not susceptible to the marker. Two types of such cells are ones that are not naturally susceptible to the marker (e.g., introduction of a ccdB counter-selectable marker into a yeast cell) or one that encodes an antidote or is otherwise resistant to the counter-selectable marker product (e.g., ccdA and ccdB).

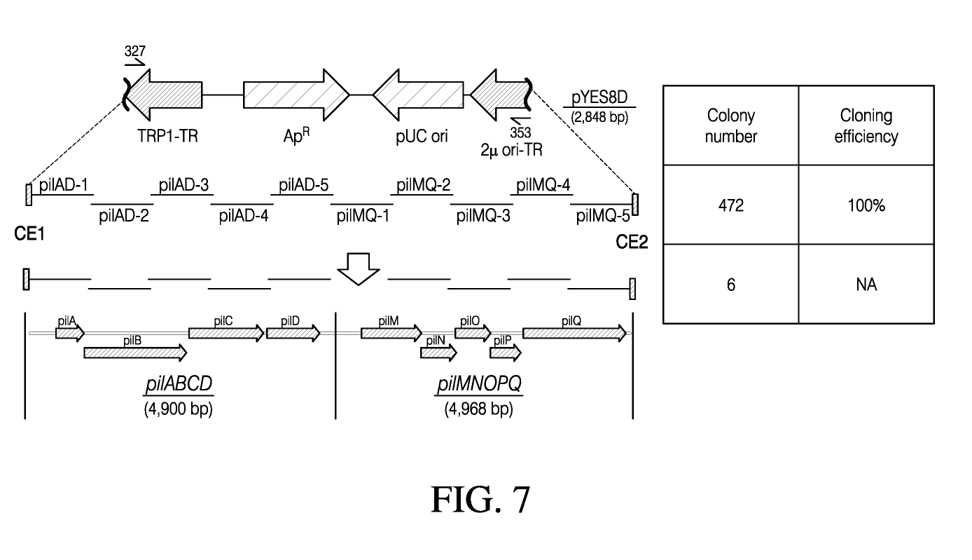

FIG. 7 shows a work flow using compositions and methods of the invention. pYES8D is employed to assemble a 9.9 kb region of the Vibrio cholera genome. As can be seen from the numerical data on the right, high efficiency assembly and cloning were achieved.

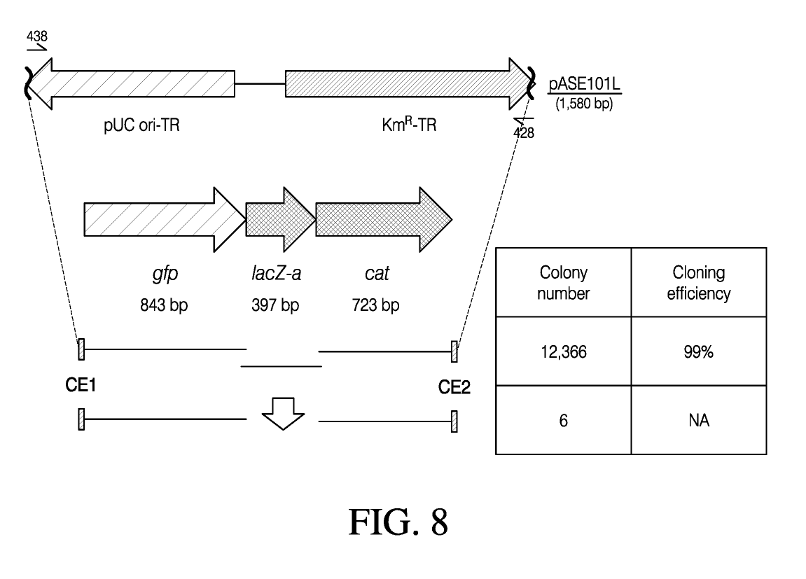

FIG. 8 shows a workflow for the assembly of and E. coli vector containing (1) an origin of replication, (2) a kanamycin resistance marker, (3) a green fluorescent protein gene fragment, (4) a lacZ-α fragment, and (5) a chloramphenicol resistance marker fragment.

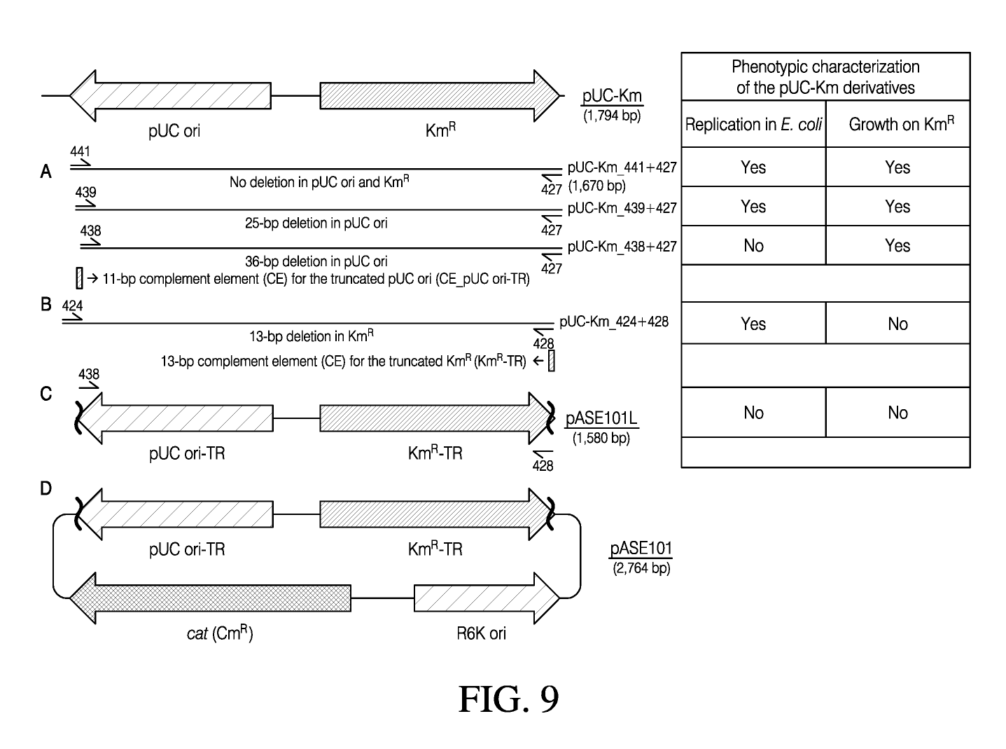

FIG. 9 shows a work flow for full assembly of an E. coli vector containing two origins of replication, one functional and the other truncated. Also present are two selectable markers, one function and the other truncated. Vectors such as this may be used to produce vector fragments suitable for use in assembly reactions. The invention thus includes compositions and methods for: (1) generating vectors suitable for use in assembly reactions and (2) vectors containing truncated functional elements for use in assembly reactions. The second item relates to method for producing truncated vector fragments for use in assembly reaction.

Error Identification and Correction:

Errors may find their way into nucleic acid molecules in a number of ways. Examples of such ways include chemical synthesis errors, amplification/polymerase mediated errors (especially when non-proof reading polymerases are used), and assembly mediated errors (usually occurring at nucleic acid segment junctions).

Two ways to lower the number of errors in assembled nucleic acid molecules is by (1) selection of nucleic acid segments for assembly with corrects sequences and (2) correction of errors in nucleic acid segments, partially assembled sub-assemblies nucleic acid molecules, or fully assembled nucleic acid molecules.

In many instances, errors are incorporated into nucleic acid molecules regardless of the method by which the nucleic acid molecules are generated. Even when nucleic acid segments known to have correct sequences are used for assembly, errors can find their way into the final assembly products. Thus, in many instances, error reduction will be desirable. Error correction can be achieved by any number of means.

One method is by individually sequencing nucleic acid segments (e.g., chemically synthesized nucleic acid segments), followed by assembly of only nucleic acid segments determined to have correct sequences. This may be done by the selection of a single nucleic acid segment for amplification, then sequencing of the amplification products to determine if any errors are present. Thus, the invention also includes selection methods for the reduction of sequence errors. Methods for amplifying and sequence verifying nucleic acid molecules are set out in U.S. Pat. No. 8,173,368, the disclosure of which is incorporated herein by reference. Similar methods are set out in Matzas et al., Nature Biotechnology, 28:1291-1294 (2010).

Another way to reduce the number of sequence errors is by error correction. An exemplary error correction workflow is set out in FIG. 10, which shows a process for synthesis of error-minimized nucleic acid molecules. In the first step, nucleic acid molecules of a length smaller than that of the full-length desired nucleotide sequence (i.e., “nucleic acid molecule fragments” of the full-length desired nucleotide sequence) are obtained. Each nucleic acid molecule is intended to have a desired nucleotide sequence that comprises a part of the full length desired nucleotide sequence. Each nucleic acid molecule may also be intended to have a desired nucleotide sequence that comprises an adapter primer for PCR amplification of the nucleic acid molecule, a tethering sequence for attachment of the nucleic acid molecule to a DNA microchip, or any other nucleotide sequence determined by any experimental purpose or other intention. The nucleic acid molecules may be obtained in any of one or more ways, for example, through synthesis, purchase, etc.

In the optional second step, the nucleic acid molecules are amplified to obtain more of each nucleic acid molecule. The amplification may be accomplished by any method, for example, by PCR. Introduction of additional errors into the nucleotide sequences of any of the nucleic acid molecules may occur during amplification.

In the third step, the amplified nucleic acid molecules are assembled into a first set of molecules intended to have a desired length, which may be the intended full length of the desired nucleotide sequence. Assembly of amplified nucleic acid molecules into full-length molecules may be accomplished in any way, for example, by using a PCR-based method.

In the fourth step, the first set of full-length molecules is denatured. Denaturation renders single-stranded molecules from double-stranded molecules. Denaturation may be accomplished by any means. In some embodiments, denaturation is accomplished by heating the molecules.

In the fifth step, the denatured molecules are annealed. Annealing renders a second set of full-length, double-stranded molecules from single-stranded molecules. Annealing may be accomplished by any means. In some embodiments, annealing is accomplished by cooling the molecules.

In the sixth step, the second set of full-length molecules are reacted with one or more endonucleases to yield a third set of molecules intended to have lengths less than the length of the complete desired gene sequence. The endonucleases cut one or more of the molecules in the second set into shorter molecules. The cuts may be accomplished by any means. Cuts at the sites of any nucleotide sequence errors are particularly desirable, in that assembly of pieces of one or more molecules that have been cut at error sites offers the possibility of removal of the cut errors in the final step of the process. In an exemplary embodiment, the molecules are cut with T7 endonuclease I, E. coli endonuclease V, and Mung Bean endonuclease in the presence of manganese. In this embodiment, the endonucleases are intended to introduce blunt cuts in the molecules at the sites of any sequence errors, as well as at random sites where there is no sequence error.

In the last step, the third set of molecules is assembled into a fourth set of molecules, whose length is intended to be the full length of the desired nucleotide sequence. Because of the late-stage error correction enabled by the provided method, the set of molecules is expected to have many fewer nucleotide sequence errors than can be provided by methods in the prior art.

The process set out above and in FIG. 10 is also set out in U.S. Pat. No. 7,704,690, the disclosure of which is incorporated herein by reference.

Another process for effectuating error correction in chemically synthesized nucleic acid molecules is by a commercial process referred to as ERRASETM (Novici Biotech). Error correction methods and reagent suitable for use in error correction processes are set out in U.S. Pat. Nos. 7,838,210 and 7,833,759, U.S. Patent Publication No. 2008/0145913 A1 (mismatch endonucleases), and PCT Publication WO 2011/102802 A1, the disclosures of which are incorporated herein by reference.

Exemplary mismatch endonucleases include endonuclease VII (encoded by the T4 gene 49), RES I endonuclease, CEL I endonuclease, and SP endonuclease or methyl-directed endonucleases such as MutH, MutS or MutL. The skilled person will recognize that other methods of error correction may be practiced in certain embodiments of the invention such as those described, for example, in U.S. Patent Publication Nos. 2006/0127920 AA, 2007/0231805 AA, 2010/0216648 A1, 2011/0124049 A1 or U.S. Pat. No. 7,820,412, the disclosures of which are incorporated herein by reference.

One error correction methods involves the following steps. The first step is to denature DNA contained in a reaction buffer (e.g., 200 mM Tris-HCl (pH 8.3), 250 mM KCl, 100 mM MgCl₂, 5 mM NAD, and 0.1% TRITON® X-100) at 98° C. for 2 minutes, followed by cooling to 4° C. for 5 minutes, then warming the solution to 37° C. for 5 minutes, followed by storage at 4° C. At a later time, T7endonuclease I and DNA ligase are added the solution 37° C. for 1 hour. The reaction is stopped by the addition EDTA. A similar process is set out in Huang et al., Electrophoresis 33:788 796 (2012).

Once nucleic acid segments are assembled, their sequences may be confirmed by sequence analysis. Sequence analysis may be used to confirm that “junction” sequences are correct and that no other nucleotide sequence “errors” are located within assembled nucleic acid molecules.

A number of nucleic acid sequences methods are known in the art and include Maxam-Gilbert sequencing, chain-termination sequencing (e.g., Sanger sequencing), pyrosequencing, sequencing by synthesis and sequencing by ligation.

The invention thus includes compositions and methods for the assembly of nucleic acid molecules with high sequence fidelity. High sequence fidelity can be achieved by several means, including sequencing of nucleic acid segments prior to assembly or partially assembled nucleic acid molecules, sequencing of fully assembled nucleic acid molecules to identify ones with correct sequences, and/or error correction.

High Order Assembly:

Large nucleic acid molecules are relatively fragile and, thus, shear readily. One method for stabilizing such molecules is by maintaining them intracellularly. Thus, in some aspects, the invention involves the assembly and/or maintenance of large nucleic acid molecules in host cells. Large nucleic acid molecules will typically be 20 kb or larger (e.g., larger than 25 kb, larger than 35 kb, larger than 50 kb, larger than 70 kb, larger than 85 kb, larger than 100 kb, larger than 200 kb, larger than 500 kb, larger than 700 kb, larger than 900 kb, etc.).

Methods for producing and even analyzing large nucleic acid molecules are known in the art. For example, Karas et al., “Assembly of eukaryotic algal chromosomes in yeast, Journal of Biological Engineering 7:30 (2013) shows the assembly of an algal chromosome in yeast and pulse-field gel analysis of such large nucleic acid molecules.

As suggested above, one group of organisms known to perform homologous recombination fairly efficient is yeasts. Thus, host cells used in the practice of the invention may be yeast cells (e.g., Saccharomyces cerevisiae, Schizosaccharomyces pombe, Pichia, pastoris, etc.).

Yeast hosts are particularly suitable for manipulation of donor genomic material because of their unique set of genetic manipulation tools. The natural capacities of yeast cells, and decades of research have created a rich set of tools for manipulating DNA in yeast. These advantages are well known in the art. For example, yeast, with their rich genetic systems, can assemble and re-assemble nucleotide sequences by homologous recombination, a capability not shared by many readily available organisms. Yeast cells can be used to clone larger pieces of DNA, for example, entire cellular, organelle, and viral genomes that are not able to be cloned in other organisms. Thus, in some embodiments, the invention employs the enormous capacity of yeast genetics generate large nucleic acid molecules (e.g., synthetic genomics) by using yeast as host cells for assembly and maintenance.

Exemplary of the yeast host cells are yeast strain VL6-48N, developed for high transformation efficiency parent strain: VL6-48 (ATCC Number MYA-3666TM)), the W303a strain, the MaV203 strain (Thermo Fisher Scientific, cat. no. 11281-011), and recombination-deficient yeast strains, such as the RAD54 gene-deficient strain, VL6-48-Δ54G (MATαhis3-Δ200 trp1-Δ1 ura3-52 lys2 ade2-101 met14 rad54-Δ1::kanMX), which can decrease the occurrence of a variety of recombination events in yeast artificial chromosomes (YACs).

Sample Preparation and Storage:

In some instances, enzymes associated with nucleic acid assembly reactions interfere with nucleic acid molecule stability. As a result, some assembly protocols call for the transformation of cells within a short time period (e.g., less than one hour) after assembly has been performed. This is not always convenient and, in some cases (e.g., high-throughput applications), may not be practical. The invention thus provides compositions and methods for stabilizing partially and/or fully assembled nucleic acid molecules.

This aspect of methods of the invention involves the use of conditions for inhibiting enzymatic reactions employed in the assembly of nucleic acid segments. One enzyme that may be inhibited is exonuclease. Exonucleases, as well as other enzymes (e.g., polymerases and ligases), may be inhibited by (1) the addition of an inhibitor, a proteinase, and/or an antibody with binding affinity for a reaction component (e.g., an exonuclease) and/or (2) physical means such as alteration of pH, metal ion concentration, heating, or salt concentration. Also, compositions and methods of the invention may involve a combination of inhibition methods. One goal of such methods is to reduce the activity of enzymatic function to a desired level, including essentially complete inactivation (i.e., unidentifiable levels of activity).

In terms of reduction of exonuclease activity, the level of inhibition will typically be measured under conditions and at a temperature (e.g., 37° C.) where the particular enzyme exhibits high levels of activity. This provides a benchmark for comparison. Exemplary reaction conditions include 67 mM glycine-KOH, 2.5 mM MgCl₂, 50 μg/ml BSA, pH 9.4, 37° C. (Lambda Exonuclease); and 67 mM glycine-KOH, 6.7 mM MgCl₂, 10 mM 3-mercaptoethanol, pH 9.5, 37° C. (E. coli Exonuclease I). Typically, the goal will be to achieve a reduction in enzymatic activity of at least 80% (e.g., at least 85%, at least 90%, at least 95%, at least 97%, at least 98%, from about 80% to about 99%, from about 80% to about 98%, from about 80% to about 97%, from about 80% to about 95%, from about 80% to about 93%, from about 85% to about 99%, from about 85% to about 98%, from about 85% to about 97%, from about 85% to about 95%, from about 90% to about 99%, from about 90% to about 98%, from about 90% to about 97%, from about 90% to about 96%, from about 90% to about 95%, from about 90% to about 94%, from about 90% to about 93%, etc.) as compared to benchmark conditions.

Methods for identifying degradation of nucleic acid molecules include transformation efficiency and gel electrophoresis. With gel electrophoresis, a portion of a reaction mixture may be run on a gel and the amount of “smearing” may be determined. The level of smearing may then be used to calculate the amount (e.g., percentage) of the nucleic acids present that have been damaged. Thus, in some aspects, assays that may be used for determining whether a sample has been stabilized by methods for the invention involve the measurement of degradation of nucleic acid molecules in reaction mixtures maintained under defined storage conditions (e.g., −20° C. for 2 weeks, −20° C. for 4 weeks, −20° C. for 8 weeks, −20° C. for 12 weeks, −20° C. for 20 weeks, −20° C. for 24 weeks, −20° C. for 30 weeks, −20° C. for 36 weeks, −20° C. for 40 weeks, −20° C. for 48 weeks, −20° C. for 52 weeks, −70° C. for 2 weeks, −70° C. for 4 weeks, −70° C. for 8 weeks, −70° C. for 12 weeks, −70° C. for 20 weeks, −70° C. for 24 weeks, −70° C. for 30 weeks, −70° C. for 36 weeks, -70° C. for 40 weeks, −70° C. for 48 weeks, −70° C. for 52 weeks, etc.).

Enzymatic reactions normally follow a trend of decreasing as the temperature decreases from the optimum temperature for the particular enzyme catalyzing the reaction. Further, enzymatic reactions continue to occur even when reactions mixtures are frozen. Also, the lower the temperature after a sample is frozen, the lower the enzymatic reaction rate. Thus, enzymatic reaction rates are expected to be lower at −70° C. than at −20° C. The benchmark temperature referenced above is used for convenience because assaying of enzymatic activity under common laboratory sample storage conditions (e.g., −20° C.) is generally more difficult than under optimal reaction conditions. Also, high levels of enzymatic activities typical associated with optimal reaction conditions (or reactions conditions close thereto) provide sufficient activity to accurately measure the effects of inhibitory conditions.

Exonuclease inhibitors that may be used in the practice of the invention include 8-oxoguanine, mononucleotides, nucleoside 5′-monophsophates, 6-mercaptopurine ribonucleoside 5′-monophsophate, sodium fluoride, fludarabine (9-β-D-arabinofuranosyl-2-fluoroadenine 5′-monophosphate)-terminated DNA, and nucleic acid binding proteins (e.g., poly(U)-binding protein. Exonuclease inhibitors may inhibit specific exonucleases, groups of exonucleases (e.g., 3′ to 5′ exonucleases, 5′ to 3′ exonucleases, etc.), or essentially all exonucleases.

As noted above, pH may also be altered to inhibit enzymatic activities (e.g., exonuclease activity). Many exonucleases, for example, exhibit significant nuclease activity at pHs in ranges of 7.5 to 9.5. A shifting of the pH away from the optimum for the particular exonuclease or exonucleases used will generally decrease enzymatic activities. Further, the farther the pH is shifted from the optimum pH, the less enzymatic activity is expected. Also, pH may be shifted higher or lower. In instances, where the removal of RNA is desired pH may be shifted higher because RNA, but generally not DNA, is hydrolyzed under basic conditions.

In many instances, pH shifts will be greater than one pH unit from the optimum pH of at least one of the exonucleases present in a nucleic acid segments assembly reaction mixture. Thus, if the optimum pH for a particular enzyme is 7.5, then the pH would be shifted to at least either pH 6.5 or 8.5. pH shifts will typically be in the ranges of from about 1 to about 7 pH units, from about 1.5 to about 7 pH units, from about 2 to about 7 pH units, from about 2.5 to about 7 pH units, from about 3 to about 7 pH units, from about 3.5 to about 7 pH units, from about 4 to about 7 pH units, from about 4.5 to about 7 pH units, from about 5 to about 7 pH units, from about 1 to about 6 pH units, from about 1.5 to about 6 pH units, from about 2 to about 6 pH units, from about 2.5 to about 6 pH units, from about 3 to about 6 pH units, from about 3.5 to about 6 pH units, from about 4 to about 6 pH units, from about 4.5 to about 6 pH units, from about 5 to about 6 pH units, from about 1 to about 5 pH units, from about 1.5 to about 5 pH units, from about 2 to about 5 pH units, from about 2.5 to about 5 pH units, from about 3 to about 5 pH units, from about 3.5 to about 5 pH units, from about 4 to about 5 pH units, from about 4.5 to about 5 pH units, etc.

Many enzymes, including exonucleases, require divalent metal ions (e.g., magnesium, manganese, and calcium) for enzymatic activity. Removal or sequestration of divalent metal ions may also be used to inhibit enzymatic activities. For example, divalent metal ion sequestration may occur by the addition of a chelating agent such as EDTA, EGTA, 1,2-bis(o-aminophenoxy)ethane-N,N,N′,N′-tetraacetic acid (BAPTA). Many chelating agents have higher affinity for some metal ions than other metal ions. For example, EGTA is more selective for calcium ions than magnesium ions.

Final divalent metal ion concentrations in exonuclease reaction mixtures, for example, tend to be in the range of 2 to 7 mM. Sequestration agents, when used, will typically be present in an amount to binding greater than 95% of the total amount of divalent metal ion present. The stoichiometry will often be determined by the affinity of the sequestration agent for the divalent metal ion, the amount of divalent metal ion present, the amount of sequestration agent present, the amount of ions present that compete for the sequestration agent, and other reaction mixture conditions. Typically, sequestration agents will be present in an amount that is at least equal to the divalent metal ion (1:1) but may be present in a greater amount (e.g., from about 5:1 to about 1:1, from about 4:1 to about 1:1, from about 3:1 to about 1:1, from about 5:1 to about 1:1, from about 5:1 to about 1:1, from about 5:1 to about 1:1, from about 2:1 to about 1:1, from about 1.5:1 to about 1:1, from about 1.25:1 to about 1:1, from about 5:1 to about 1.1:1, from about 2.5:1 to about 1.1:1, from about 5:1 to about 1.5:1, from about 2.5:1 to about 1.5:1, from about 5:1 to about 2:1, from about 4:1 to about 2:1, from about 5:1 to about 1.5:1, etc.). In many instances, the amount of sequestration agent will be adjusted to achieve a reduction in enzymatic activity of at least 80% under the selected benchmark conditions.

One method of inhibiting thermolabile enzymes (e.g., exonucleases, ligases and polymerases) is by heating aqueous reaction mixtures (e.g., aqueous reaction mixtures) containing these enzymes for a sufficient period of time to allow for enzymatic inactivation. In most instances, this will result in irreversible inactivation by denaturation of enzyme(s) present in the reaction mixtures. Suitable heating conditions will vary with the thermal properties of particular enzymes present but will generally be greater than 60° C. (e.g., from about 60° C. to about 95° C., from about 65° C. to about 95° C., from about 70° C. to about 95° C., from about 75° C. to about 95° C., from about 80° C. to about 95° C., from about 60° C. to about 90° C., from about 60° C. to about 85° C., from about 60° C. to about 80° C., from about 60° C. to about 75° C., from about 65° C. to about 90° C., from about 60° C. to about 95° C., from about 65° C. to about 85° C., from about 70° C. to about 95° C., from about 70° C. to about 90° C., etc.) for at least 5 minutes (e.g., from about 5 min. to about 30 min., from about 5 min. to about 20 min., from about 5 min. to about 15 min., from about 5 min. to about 10 min., from about 10 min. to about 30 min., from about 10 min. to about 25 min., from about 10 min. to about 20 min., etc.).

One advantage of heating to inactivate exonucleases is that, in many instances, it will not be necessary to open containers (e.g., tubes) or add reagents as part of the inactivation step. This is especially useful when high-throughput methods are used.

Another way in which assembly reactions may be inhibited is through degradation of one or more assembly reaction components (e.g., an exonuclease). This may be done, for example, using a one or more proteinase. Exemplary proteinases include serine endopeptidases (e.g., Proteinase K of Tritirachium album limber) and aspartate proteinases (e.g., pepsin and cathepsin D), threonine proteases, cysteine proteases, glutamic acid proteases, and metalloproteases. Thus, the invention includes methods in which assembled nucleic acid molecules are exposed to one or more proteinase for a time sufficient to inhibit assembly reaction components.

Inhibition of assembly reaction components may be measure in a number of ways. One way is by measure the reduction in one or more assembly reaction activity (e.g., exonuclease or ligase activity). For example, when inhibition of exonuclease activity is measured, the amount of reduction of activity is discussed above but will often be greater than 75%. Further, this reduction in activity may be measured in units, with, for example, a decrease in activity of at least 75 units as compared to a control.

Exonuclease units may be defined as the amount of enzyme that will catalyze the release of 10 nanomole of acid-soluble nucleotide in 30 minutes at 37° C. in a total reaction volume of 50 μl, with the reaction mixture containing 67 mM Glycine-KOH, 6.7 mM MgCl₂, 10 mM (3-ME, pH 9.5 at 25° C. and 0.17 mg/ml single-stranded [³H]-DNA.

Methods for assessing exonuclease activity based on the preferential binding of single-stranded DNA over double-stranded DNA to graphene oxide are set out, for example, in Lee et al., “A simple fluorometric assay for DNA exonuclease activity based on graphene oxide,” Analyst 137:2024-2026 (2012).

Another way in which assembly reactions can be inhibited is through the use of antibodies with binding affinity for assembly reaction components (e.g., ligase and exonuclease). A number of antibodies with binding affinity for, for examples, ligases and exonucleases are commercially available from companies such as abcam (1 Kendall Square, Suite B2304, Cambridge, Mass. 02139), including Anti-DNA Ligase III antibody [6G9] (ab587), Anti-DNA Ligase I antibody [10H5] (ab615), Anti-DNA Ligase IV antibody (ab26039), and Anti-Exonuclease 1 antibody (ab106303).

More than one (e.g., two, three or four) enzyme (e.g., exonuclease) inhibition method may be used in the practice of the invention. For example, a pH shift may be use in conjunction with heating. When a thermostable enzyme is used, heat based inactivation will generally not be used.

The invention thus provides compositions and methods for stabilizing assembled nucleic acid molecules present in reaction mixtures. These reaction mixtures will generally contain components (e.g., enzymes) that can cause damage to the nucleic acid molecules present therein. Nucleic acid molecules in reaction mixtures prepared using methods of the invention will typically show little (less than 5% of the total nucleic acid molecules present) or no degradation upon storage at −20° C. for 8 weeks, −20° C. for 12 weeks, −20° C. for 24 weeks, −70° C. for 12 weeks, −70° C. for 24 weeks, −70° C. for 36 weeks, or -70° C. for 52 weeks.

Kits:

The invention also provides kits for the assembly and storage of nucleic acid molecules. As part of these kits, materials and instruction are provided for both the assembly of nucleic acid molecules and the preparation of reaction mixtures for storage.

Kits of the invention will often contain one or more of the following components:

1. One or more exonuclease,

2. One or more polymerase,

3. One or more ligase,

4. One or more partial vector (e.g., one or more nucleic acid segment containing an origin of replication and/or a selectable marker) or complete vector,

5. One or more enzymatic (e.g., an exonuclease) inhibitor (e.g., a solution with a pH above 9 or below 6.5, a sequestration agent, and, optionally, one or more of the following

6. One or more non-vector nucleic acid segments in may

7. Instructions for how to prepare and store samples (e.g., direction the addition of one or more inhibitory compound and/or heating of the sample, followed by storage at low temperature (e.g., −20° C. or below).

### EXAMPLES

### Example 1

### Seamless Cloning Using Phosphorothioate Chemistry

There is increasing demand for large, high-fidelity, synthetic DNA constructs. However, the most commonly synthesized genes range in size from 600 to 1,200 bp. Further seamless assembly is required to obtain large nucleic acid (e.g., DNA) constructs. A seamless, sequence-independent nucleic acid assembly method, based on phosphorothioate chemistry, is set out in this example. Some features of methods set out in this example are:

1. The use of phosphorothioate chemistry stops the “chew back” reaction of exonuclease at a specified location, allowing the generation of controllable overhangs and correct assembly.

2. Synthetic DNA fragments are generated by PCR using a pair of phosphorothioate end primers, followed by one-step reaction using, for example, the GeneArt® Seamless Cloning and Assembly Kit (Life Technologies Corporation, now part of Thermo Fisher Scientific, cat. no. A13288).

3. Data indicate that the efficiency of cloning ten 1 kb PCR fragments is around 98%, with about 2000 colonies, although the efficiency of cloning ten synthetic strings reduces to about 64%.

4. DNA sequencing analysis confirms the integrity of the DNA conjunctions.

5. Optimization of assembly reactions can be achieved by the alteration of factors such as PCR conditions, length of overhangs, amount of DNAs, and incubation times. In brief, these are highly efficient in vitro assembly methods applicable, for example, to gene synthesis.

Introduction: Long synthetic DNA fragments (e.g., >10 kb), commonly used for the construction of large genes and multi gene pathways, are often challenging to assemble. Traditional restriction-based ligation methods are sequence-specific and often generate “scars”.

Homologous recombination-based methods, such as those employed by the GeneArt® Seamless Cloning and Assembly Kit (Life Technologies Corporation, now part of Thermo Fisher Scientific, cat. no. A13288), utilize exonuclease to generate single-stranded DNA overhangs for joining of overlapping fragments. However, the “chew back” reaction is often difficult to control, which leads to non-specific annealing amongst DNA fragments and decreases the efficiency of large DNA assembly.

In this example, a highly efficiency DNA assembly methods is described, which utilizes phosphorothioate chemistry in conjunction with GeneArt® Seamless Cloning and Assembly Enzyme Mix (cat. no. A14606). These methods allow for one-step assembly of, for example, ten 1 kb PCR fragments, as well as repetitive DNA fragments.

Material and Methods.

Materials: Phusion DNA polymerase (NEB), GeneArt® Seamless Cloning and Assembly Kit (Life Technologies Corporation, now part of Thermo Fisher Scientific, cat. no. A13288), AccuPrime™ Pfx DNA polymerase (Thermo Fisher, cat. no. 12344-032), T4 DNA ligase (Thermo Fisher, cat. no. 15224-090), PureLink™ Quick PCR purification kit (Thermo Fisher, cat. no. K3100-1), pType-IIs recipient vector (vector map can be viewed at www.lifetechnologies.com), One-Shot TOP10 Chemically Competent Cells (Thermo Fisher, cat. no. C4040-10), BigDye terminator v3.1 cycle sequencing kit (Thermo Fisher, cat. no. 4337457), E-gel (Thermo Fisher cat. no. G5018-8), synthetic DNAs and the trimers of Tal assembly repeats are synthesized by GeneArt® (Thermo Fisher).

Methods:

Oligo Design: Two adjacent PCR fragments share 15 bases of homology at each end (FIG. 2). Two consecutive oligonucleotides modified by a phosphorothioate (PS) linkage were added to positions 16 and 17 accounting from the 5′ end. Typically, the phosphorothioate primer is approximately 20-30 nucleotides long. For assembly of repetitive DNA fragments, the adjacent DNA fragments can have a 12-bp overlap at their ends, in which the two PS bonds are positioned at nucleotides 13 and 14, counting from the 5′ end.

The following phosphorothioate primers were used for DNA amplification and assembly:

TABLE 2
Mycoplasma genitalium

Frag1-F2-5kb:
TGC TGG AGT GAA CGC ZEG GCC GAG CGC AAA G (SEQ ID
NO: 1)
Frag1-R-5kb:
GCA AGA AAA CTA TCC OEA CCG CC (SEQ ID NO: 2)
Frag2-F-5kb:
GGA TAG TTT TCT TGC EEC CCT AAT C (SEQ ID NO: 3)
Frag2-R-5kb:
CGT CTG GGA CTG GGT EEA TCA G (SEQ ID NO: 4)
Frag3-F-5kb:
ACC CAG TCC CAG ACG FFG CCG C (SEQ ID NO: 5)
Frag3-R-5kb:
CAG ATG TGC GGC GAG ZZG CGT GAC TAC (SEQ ID NO: 6)
Frag4-F-5kb:
CTC GCC GCA CAT CTG FFC TTC AGC (SEQ ID NO: 7)
Frag4-R-5kb:
CGC AGT GGA AGA TAG FZC TGA TTG (SEQ ID NO: 8)
Frag5-F-5kb:
CTA TCT TCC ACT GCG FET TGA A (SEQ ID NO: 9)
Frag5-R-10kb:
AGT GCA GTT GGT GGA EZT GTT GAT G (SEQ ID NO: 10)
Frag6-F-10kb:
TCC ACC AAC TGC ACT FEG AGA TTG (SEQ ID NO: 11)
Frag6-R-10kb:
AGC AAG GTG AGA TTG FFA CTA GGA TTG (SEQ ID NO: 12)
Frag7-F-10kb: CAA TCT CAC CTT GCT EZG CTT TAG C
(SEQ ID NO: 13)
Frag7-R-10kb:
TCT TGC CCT AGC AGT ZEG TCA TAC CAA C (SEQ ID NO: 14)
Frag8-F-10kb:
ACT GCT AGG GCA AGA FOC ACC ACC AAA TAG (SEQ ID
NO: 15)
Frag8-R-10kb:
CTT TAG ATG GTG AGA OFG TTT ATG CAG G (SEQ ID NO: 16)
Frag9-F-10kb:
TCT CAC CAT CTA AAG ZFA CGA TCC (SEQ ID NO: 17)
Frag9-R-10kb:
CTG TTG GGT TAG ATC FFA TGG CG (SEQ ID NO: 18)
Frag10-F-10kb:
GAT CTA ACC CAA CAG ZFG GTT C (SEQ ID NO: 19)
Frag10-R-10kb:
CAC ATG CCT CCC TTT ZOC ACT TTT ATT G (SEQ ID NO: 20)
pLP-F-10kb:
AAA GGG AGG CAT GTG FEC AAA AGG (SEQ ID NO: 21)
pLP-R2:
GCC CAG CGT TCA GGC OEC GAT ATC ACC C (SEQ ID NO: 22)
DNA IUPAC 1-Letter Codes:
F = phosphorothioate-A base; O = phosphorothioate-C base; E = phosphorothioate-G base; Z = phosphorothioate-T base.

TABLE 3
Synthetic Strings

String1-F2: GGC CTA AAA GAC TCT FFC AAA ATA GCA AAT TTC G (SEQ ID NO: 23)
String1-R: CCC ATT AGG CCA TTT OFG CAG (SEQ ID NO: 24)
String2-F: AAA TGG CCT AAT GGG ZZA CGA TGC TTT GTT CTT G (SEQ ID NO: 25)
String2-R: ACC TCT CCA ATA ATT ZET TCC AAG TAA CCA TCT TCA C (SEQ ID NO: 26)
String3-F: AAT TAT TGG AGA GGT ZET GTT GCT GAA GGT G (SEQ ID NO: 27)
String3-R: GCT TCA CCC ACA AAG OOA ATC TAG CAC (SEQ ID NO: 28)
String4-F: CTT TGT GGG TGA AGC ZEA TAG AGG TGA TG (SEQ ID NO: 29)
String4-R: TCT GGT CAT CTC TCA FOA ACA AAT CAC CC (SEQ ID NO: 30)
String5-F: TGA GAG ATG ACC AGA ZZT GGG TGC TAA ATT GCC (SEQ ID NO: 31)
String5-R: GTT CAG CAG TTC TCT ZOT TCT ATC ACC AG (SEQ ID NO: 32)
String6-F: AGA GAA CTG CTG AAC FFT TAC AAT TGG C (SEQ ID NO: 33)
String6-R: TTC TAG CCA AGG TTC OFA CAT GGA GGC (SEQ ID NO: 34)
String7-F: GAA CCT TGG CTA GAA EFT GTG AAA GAT TAT TGG (SEQ ID NO: 35)
String7-R: AAC CAG AAA GGC TCT OFT AGT AGG (SEQ ID NO: 36)
String8-F: AGA GCC TTT CTG GTT OZC CAT CTT TGA C (SEQ ID NO: 37)
String8-R: CTC AAA GCC GAA TCT EFT GGC AAT ACC TTG (SEQ ID NO: 38)
String9-F: AGA TTC GGC TTT GAG FEA TAA GTG TAG ATC (SEQ ID NO: 39)
String9-R: CCA ATA AGA CAG TAA OOA GAA GTC AAT T (SEQ ID NO: 40)
String10-F: TTA CTG TCT TAT TGG ZOA CCA ATG TTG CC (SEQ ID NO: 41)
String10-R: CAC ATG CTA TAG AAC OOG AAC GAC CGA GC (SEQ ID NO: 42)
pLP-F-string: GTT CTA TAG CAT GTG FEC AAA AGG CCA GC (SEQ ID NO: 43)
pLP-R2-string: AGA GTC TTT TAG GCC EOG ATA TCA CCC CTA (SEQ ID NO: 44)

TABLE 4
Tal Trimers

Tal-F1f: CGC GGA ACC TGA OOC CCG AAC (SEQ ID NO: 45)
Tal-F1r: CAG TCC GTG AGC OZG GCA CAG C (SEQ ID NO: 46)
Tal-F2f: gctcacggactgFOccccg (SEQ ID NO: 47)
Tal-F2r: TCA GCC CGT GAG OOT GGC AC (SEQ ID NO: 48)
Tal-F3f: ctcacgggctgaOOcccg (SEQ ID NO: 49)
Tal-F3r: CGG GGG TCA AAC OET GAG CCT G (SEQ ID NO: 50)
Tal-F4f: gtttgacccccgFFcagg (SEQ ID NO: 51)
Tal-F4r1plus4: TGT GAG GCC GTG FEC CTG GC (SEQ ID NO: 52)
V-Tal-F1plus4: CAC GGC CTC ACA ZET GAG CAA AAG G (SEQ ID NO: 53)
V-Tal-R: TCA GGT TCC GCG FZA TCA CCC CTA (SEQ ID NO: 54)

Assembly Method: Ten 1 kb DNA fragments from either M. genitalium, V. cholerae or C. violaceum were PCR-amplified using phosphorothioate primers in the presence of either Phusion® DNA polymerase or AccuPrime™ Pfx DNA polymerase. To assemble synthetic DNA strings, synthetic DNAs were PCR-amplified using phosphorothioate primers. Linearized of pType IIs vector was also prepared by PCR amplification using phosphorothioate primers accordingly. PCR fragments were purified using standard PCR column. If the DNA concentration is too low (below 50 ng/μl), the DNA fragments can be mixed and concentrated using a Speed Vac. The DNA fragments were resuspended in 7 μl water. In a 10 μl assembly reaction, 75 ng of linear vector, 75 ng each of 10 PCR fragments, 2 μl of 5× reaction buffer, and 1 μl of 10×enzyme mix were added. The reaction was initiated by the addition of enzyme mix, followed by incubation at room temperature for 1 hour. 3 μl of reaction mix was transformed into TOP10 competent cells and then incubated on ice for 30 minutes, followed by heat shock at 42° C. for 30 seconds. Upon incubation on ice for 2 minutes, 250 μl of SOC medium was added to the transform reaction and incubated at 37° C. for 1 hour. One hundred μl of cell suspension was spread on LB+Amp plates and incubated at 37° C. overnight. Colonies were randomly picked and subjected to plasmid DNA isolation, followed by analysis of both restriction enzyme digestion and sequencing.

Results and Discussion: Because the phosphorothioate bonds stop the chew back reaction catalyzed by exonucleases at a specified location and generate perfect overhangs for homologous recombination, it was expected that the efficiency for DNA assembly would be higher than for assembly reactions using molecules not having phosphorothioate bonds, especially for large fragment assembly. To examine this, two sets of ten 1 kb fragments were designed that are PCR-amplified from either M. genitalium and V. cholerae using phosphorothioate primers, respectively. The DNA fragments share 15 bp homology at their ends. The assembly of ten 1 kb fragments plus linear vector was performed in triplicate as described above. The DNA fragments of M. genitalium also harbor a functional LacZ gene which was intentionally split into two adjacent fragments so that blue colonies were produced on X-gal plates once the DNA fragments were assembled correctly. As depicted in Table 5, about 2000 colonies per transformation were obtained. The cloning efficiency was more than 98% based on the calculation of percentage of blue colonies. To confirm the identity of the construct, 11 blue colonies were picked. Plasmid DNA was isolated from each of these colonies for restriction digestion analysis and sequencing analysis. Digestion of the 11 plasmids with BglII all generated three expected sizes of DNA fragments, which are 640 bp, 2003 bp and 8743 bp (data not shown), respectively. Sequencing of three individual plasmids reveals that all three constructs had the correct sequences at the 11 junctions connecting the fragments and vector. Similar results were observed with the second set of ten 1 kb DNA fragments that were amplified from V. cholerae. As shown in Table 6, around 2000 CFU were obtained in two individual experiments. Ten colonies were randomly picked and subjected to restriction analysis with NcoI. Upon digestion, all ten clonal isolates showed the expected sizes of DNA fragments, which are 1263 bp and 10396 bp (data not shown), respectively.

TABLE 5
One step assembly of 10 × 1 kb plus vector
using phosphorothioate primers

| | | | |
|---|---|---|---|
| No. of White Colonies | 30 | 18 | 54 |
| No. of Blue Colonies | 1884 | 2172 | 1962 |
| % of Blue Colonies | 98.4% | 99.2% | 97.3% |

| | |
|---|---|
| AVG | 98.3% ± 0.9 |

TABLE 6
Assembly of ten 1 kb fragments from V. cholerae

Exp#

| | 1 | 2 |
|---|---|---|
| CFU/rxn | 2280 | 1980 |
| CE | 100% | 100% |

Next this method was evaluated on the assembly of ten synthetic DNA fragments (strings). The synthetic strings were produced by GeneArt (Thermo Fisher) and PCR amplified using phosphorothioate primers. The quality of the PCR products was fair as some of the DNA fragments had minor truncated products. Average of 248 colonies was observed in the triplicate experiments (Table 7). Restriction digestion analysis with XmnI produced three expected sizes of DNA fragments of 1563 bp, 2317 bp and 6 kb (data not shown), suggesting that the efficiency of assembly is around 60%.

TABLE 7
Assembly of ten synthetic strings using PS primers

| | Synthetic Strings | | |
|---|---|---|---|
| | 1 | 2 | 3 |
| CFU/rxn | 225 | 186 | 333 |

| | |
|---|---|
| Avg | 248 ± 130 |
| CE | 60% |

The feasibility of using this PS approach for assembly of repetitive DNA fragments was also examiner. Tal repeat trimers having more than 90% homology were obtained from GeneArt® (Thermo Fisher). To minimize the cross-reactivity, the length of overlap was reduced from 15 bp to 12 bp. Four trimers of Tal repeats were PCR amplified using phosphorothioate primers and assembly simultaneously to produce a Tal effector containing 12 repeats. Around 28,000 colonies were observed. Five colonies were randomly picked for DNA sequencing. The results indicated that 4 out of 5 contained all four trimers of Tal repeats.

In conclusion, a robust assembly method was developed using phosphorothioate chemistry. Since T7exo hydrolyzes double stranded DNA from 5′ to 3′, it generates a 5′ phosphate at a specified phosphorothioate nucleotide. Upon annealing to a complimentary strand, the double stranded DNA contains a nick bounded by 3′-OH and 5 ′-P termini. Ligase may be used to seal the gaps.

### Example 2

### Positive Selection Assembly and Cloning

Summary: Here, a technique based on positive-selection vectors is presented. The strategy relies on vectors with a truncated and inactive replication origin and selection marker, whose short missing sequences are provided in trans during the cloning procedure. The approach i) provides selective survivability on the assembly products that have correct assembled outermost fragments and ii) reduces background colony growth due to recircularized vectors.

Materials and Methods

Strains: Chemically or electro competent Escherichia coli strains, DH10B-T1 and TOP10, were obtained from Thermo Fisher Scientific. E. coli strain S17-1::λ-pir (de Lorenzo and Timmis, Analysis and construction of stable phenotypes in gram-negative bacteria with Tn5- and Tn10-derived minitransposons, Methods Enzymol. 235:386-405 (1994)) was used to maintain the positive-selection vector pASE101. Chemically competent yeast MaV203 strain (a part of the GeneArt® High-Order Genetic Assembly System kit) was obtained from Thermo Fisher Scientific. E. coli strains were grown in LB medium appropriate antibiotics: ampicillin (Ap, 50 μg/ml), kanamycin (Km, 25 μg/ml), and chloramphenicol (Cm, 20 μg/ml). Yeast MaV203 transformants were grown on CSM-Trp medium.

Oligonucleotides, synthetic DNAs, and plasmids: Oligonucleotides used in this study are listed in Table 8. Synthetic DNA strings were obtained from Thermo Fisher Scientific (GeneArt, Germany). A subset of these synthetic DNA fragments were cloned into pCR®-Blunt II-TOPO® (Thermo Fisher Scientific) Vector as indicated below. These pre-cloned DNA fragments were used as templates to produce PCR-amplified inserts. Then those three different types of DNA, synthetic, pre-cloned, and PCR-amplified, were used for DNA assembly tests. All DNA fragments for assembly test were listed in Table 9.

A 4,255-bp DNA fragment was amplified from pYES3/CT (Thermo Fisher Scientific) using a primer set (CH316 & CH371) and circularized by self-ligation to generate pYES8. A 2,848-bp linear positive-selection vector pYES8D for in vivo DNA assembly in yeast was PCR amplified from pYES8 using a primer set (CH327 and CH353), and was also circularized by self-ligation to maintain in E. coli. Three DNA fragments, 2micron ori-TR_pUC ori (1045 bp, CH353 & CH397) and TRP1-TR (871 bp, CH399 & CH401) from pYES8D and Km^{R} (1006 bp, CH396 & CH400) from pCR®-Blunt II-TOPO® Vector (ThermoFisher), were assembled using GeneArt® Seamless PLUS Cloning and Assembly Kit (ThermoFisher) to generate pYES10. A 1815 bp DNA fragment harboring pUC ori and ApR gene from pYES8 was amplified by PCR (CH423 & CH418) and self-ligated to produce pUC-Ap. A 1794 bp DNA fragment harboring pUC ori and ApR gene from pYES10 was amplified by PCR using a primer set (CH423 & CH418) and self-ligated to produce pUC-Km. A 1581 bp DNA fragment harboring truncated pUC ori (pUC ori-TR) and Km^{R} (Km^{R}-TR) was amplified from pUC-Km by PCR using a primer set (CH428 & CH438) and assembled with a 1223 bp PCR-amplified (CH450 & CH451) synthetic DNA fragment using GeneArt® Seamless PLUS Cloning and Assembly Kit (ThermoFisher) to generate pASE101. This vector can be maintained only in an E. coli strain harboring pir gene such as S17-1::λ-pir. A linear 1581 bp positive selection vector pASE101L was amplified from pASE101 by PCR using a primer set (CH428 & CH438). A linear 1603 bp control vector pASE_cont harboring functional pUC ori and Km^{R} was amplified from pASE101 by PCR using a primer set (CH476 & CH477). Phosphorothiate version of pASE101L and pASE_cont were amplified using phosphorothioate primer sets, CHPT1 & CHPT2 and CHPT3 & CHPT4.

DNA assembly: For in vivo assembly in yeast, the protocol for GeneArt® High-Order Genetic Assembly System (ThermoFisher) was followed using a modified amount of vector (50 ng) and inserts (50 ng each). For in vitro assembly and cloning in E. coli, both GeneArt® Seamless Cloning and Assembly Kit and GeneArt® Seamless PLUS Cloning and Assembly Kit (ThermoFisher) were used following the manufacturer's protocol using vector (75 ng) and inserts (75 ng each).

Results and Conclusions

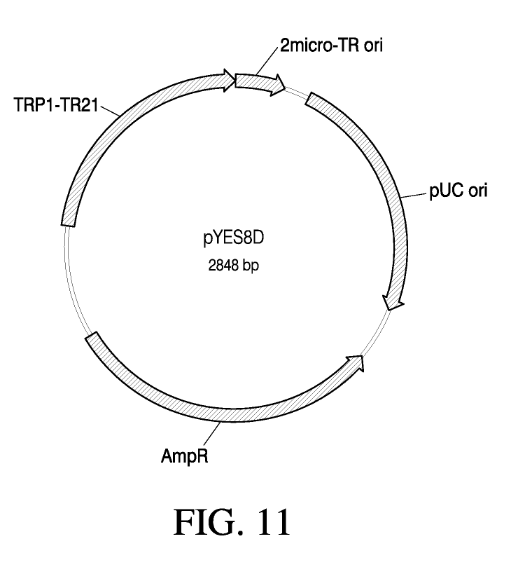

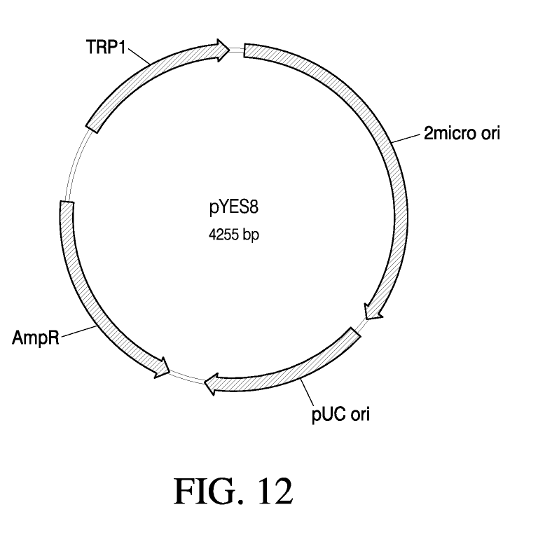

Positive selection in Saccharomyces cerevisiae: A map and sequence of the 2848 bp vector pYES8D is shown in FIG. 11 and Table 10. The plasmid encodes i) the β-lactamase gene and the replication origin (ori) from pUC19, ii) an inactive S. cerevisiae trpl gene (Braus et al., The role of the TRP1 gene in yeast tryptophan biosynthesis, J. Biol. Chem. 263:7868-75 (1988)), and iii) a truncated ori from the yeast 2μ episome (Ludwig and Bruschi, The 2-micron plasmid as a nonselectable, stable, high copy number yeast vector, Plasmid 25:81-95 (1991)). Whereas the trp1 gene misses the last 21 bp of the otherwise active wild type open reading frame, the truncated 2μ ori lacks 10 bp (AGATAAACAT) (SEQ ID NO: 55) sufficient to provide full functionality (positions 1358 to 1367 of nucleotide sequence of pYES8, the wild-type counterpart (FIG. 12 and Table 11). The plasmid is PCR amplified with divergent oligonucleotides that anneal in between the inactive elements described above (position 2684 of its nucleotide sequence, Table 10) resulting in a linear vector ready to be used for cloning in yeast.

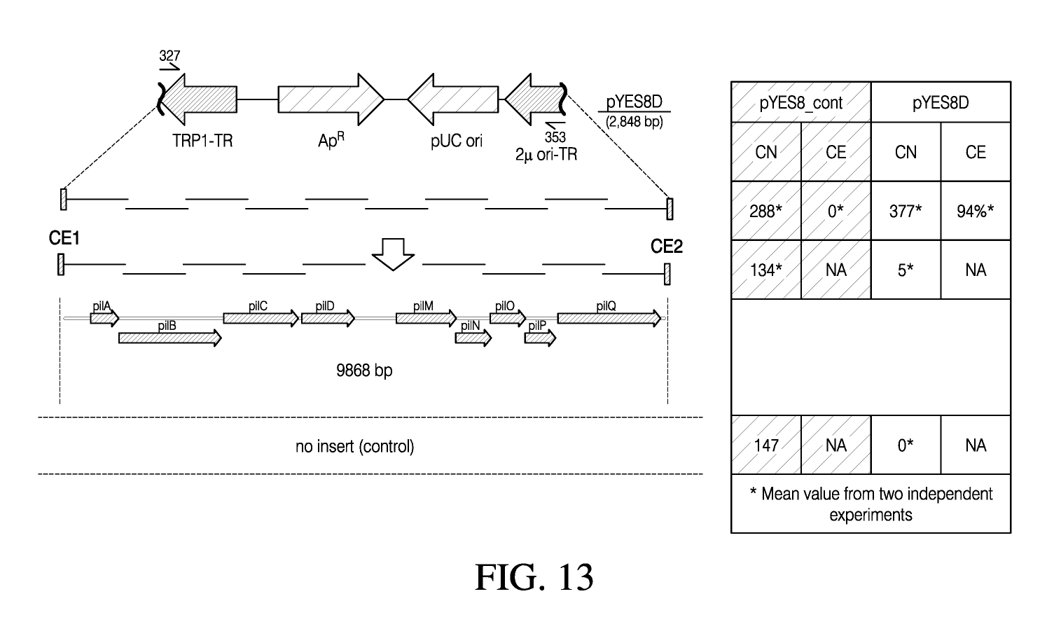

In a first cloning example 10 different fragments accounting for a total of 9868 bp were PCR amplified from Vibrio cholerae's genomic DNA and mixed with the linearized vector above (FIGS. 13 and 6). Adjacent fragments share 30 bp of homology at their corresponding ends for recombination. It is important to note that the first and 10th fragment contain the missing sequences of the truncated trp1 and 2μ ori at their 5′ and 3′ ends respectively plus 30 additional nucleotides required for recombination into the linearized vector. These additional sequences were added to the corresponding PCR primers resulting in 71 and 60 mer oligonucleotides respectively (pilAD-1 and PilMQ-5 in Table 8).

The fragments and vector were transformed into competent MaV203 yeast cells, which were subsequently plated onto CSM-Trp agar plates as indicated in materials and methods. The cells are unable to grow on media lacking tryptophan, unless they are complemented by a plasmid harboring an active trpl gene.

A series of control experiments were performed. First, a linear plasmid with intact and functional trpl and 2μ ori elements, pYES8 (FIG. 12) was used instead of pYES8D. This plasmid is not subjected to positive selection, as it does not require complementing sequences for selection, replication and maintenance. Second, a DNA array lacking fragment number 6 was used instead of the otherwise complete 10-fragment set. In this case, a construct could not be assembled, as the necessary co-linearity for homologous recombination is broken. Finally, a vector only control was included for background growth assessment.

The results showed that the positive selection vector pYES8D promoted the recombination of the expected construct with an efficiency of 94%. In other words, 94 out of 100 colonies contained the right clone. In the absence of the positive selection feature, no correct clone could be obtained, despite the fact that a comparable number of colonies appeared on the plates. Lastly, the negative control experiments (no fragment number 6 and no insert controls) produced a significantly reduced number of colonies only if the positive control vector was employed.

In a second example, 10 synthetic DNA fragments were in vitro synthesized employing standard gene synthesis procedures (FIG. 6). In this case, the homology between adjacent fragments and between the outermost fragments and the vector was introduced during the gene synthesis procedure. Three different DNA sources were employed. First, the fragments were used as they were received from the DNA synthesis provider (FIG. 6, synthetic DNA). Second, the fragments were individually cloned into a carrier vector and then released by restriction endonuclease digestion procedures (FIG. 6, pre-cloned). And third the fragments were PCR amplified from the pre-cloned constructs above (FIG. 6, PCR product). Again, a series of negative controls were used where either the first, second, fourth, or tenth fragment was excluded from the assembly procedure. Additional experiments included the pYES8 vector as described above, and two no-insert control reactions.

The results showed that with the use of the positive selection vector pYES8D, the expected final construct could be obtained with cloning efficiencies ranging from 77 to 100%. Without positive selection, the expected clone was obtained at a significantly lower rate (compare assemblies 1 and 9 in FIG. 6). Negative controls showed considerably lower colony counts.

In conclusion, the positive selection vector approach in yeast significantly reduces the downstream screening effort compared with standard selection procedures, shortening the hands-on and overall time required to obtain the expected clone.

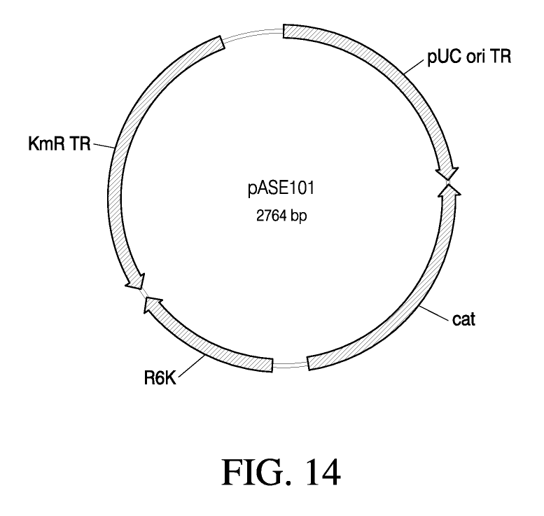

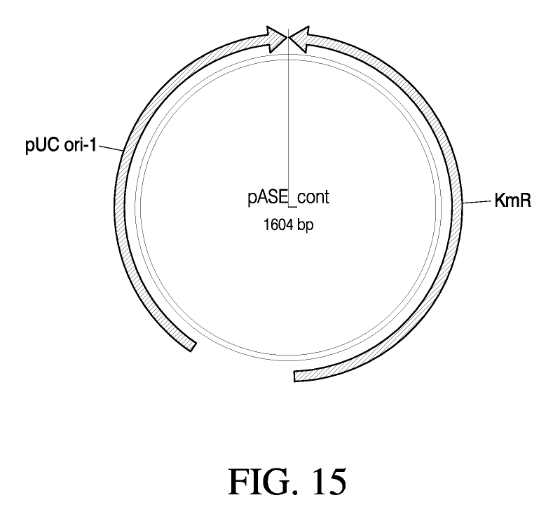

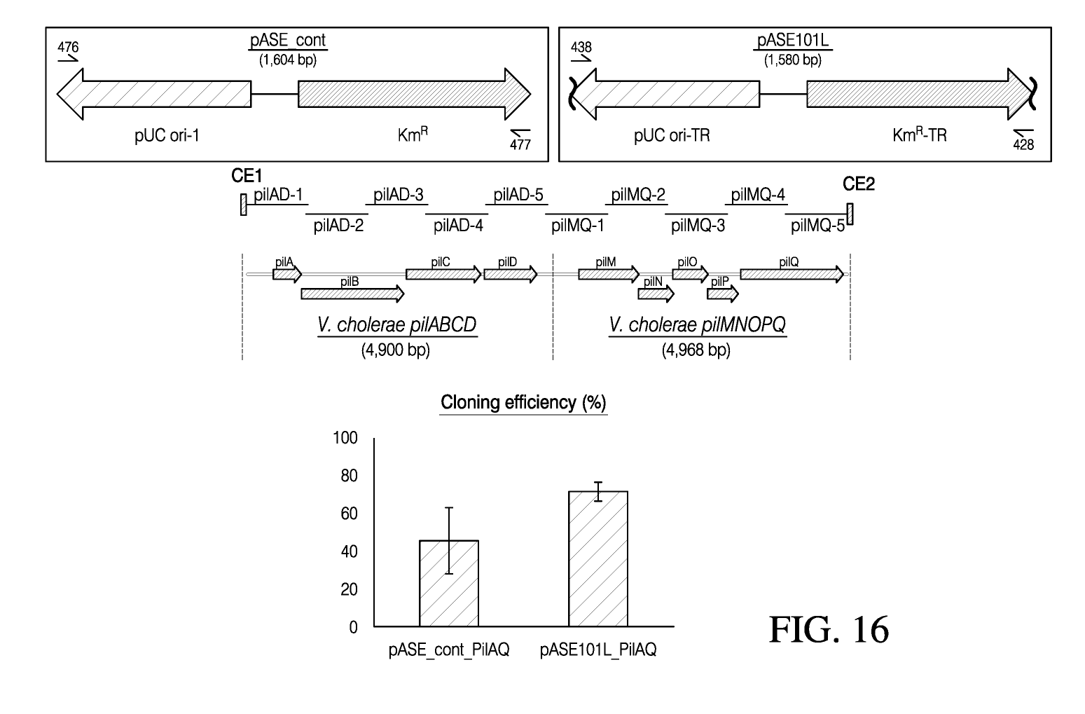

Positive selection in Escherichia coli: In this second example, the performance of the positive selection approach is shown in the context of E. coli cloning. In this case the vector, pASE101 (FIG. 14 and Table 12) harbors truncated non-functional pUC ori (pUC ori-TR) and kanamycin resistance elements (Km^{R}-TR), with 11-bp and 13-bp deletions respectively (compare sequences in FIG. 15 and Table 13). The vector pASE101 harbors a functional chloramphenicol resistance gene and the R6K ori, which restrict its propagation in pir+E. coli strains such as S17-1::λ-pir. Therefore, standard E. coli K12, W, or B strains are non-viable in the presence of selection pressure. A subfragment of the vector encompassing only pUC ori-TR and Km^{R}-TR was PCR amplified using phosphorothioated oligonucleotides, as described in the materials and method section (FIGS. 9 and 16). This fragment was used as an acceptor for the cloning reactions described below.

A similar 10-fragment array as that one described in the previous section was employed as a source of inserts. In this particular case the fragments were PCR amplified using oligonucleotides harboring phosphorothioate bonds as described in materials and methods. The construct was assembled using the GeneART Seamless Assembly kit (Thermo Fisher Scientific), and transformed into TOP10 cells (Thermo Fisher Scientific). As a control, a similar construct was assembled using the vector pASE_cont (FIG. 16), which encodes functional pUC ori and kanamycin resistant markers (no positive selection).

The results show that the positive control vector strategy significantly increases the cloning efficiency compared with the approach where no positive selection is employed (cloning efficiencies of 71 and 45% respectively).

In conclusion, the positive selection approach can be applied to the most common E. coli-based cloning complementing and boosting the performance of otherwise standard cloning methodologies.

TABLE 8
Oligonucleotides used in this study.

| | | Relevant | |
|---|---|---|---|
| | | DNA | |
| | | fragment or | SEQ ID |
| Name | Sequence (5′ to 3′) | construct | NO: |
| CH312 | TAGGCCATTTCAGCAGAAATATCTGGCAAG | Vio-1 | 56 |
| CH313 | TCTTATTGGTCACCAATGTTGCCAGAC | Vio-10 | 57 |
| CH314 | TTATCTCTTAGCAGCAAAAACAGCATCTG | Vio-10 | 58 |
| CH316 | CCAAAGCTTCAGGGGATAACGCAGGAAAGAAC | pYES8 | 59 |
| CH317 | TTGAAGCTTTCTGATTATCAACCGGGGTGGAGCTTC | pYES8 | 60 |
| CH327 | GAAATTTGCTATTTTGTTAGAGTCTTTTACACCATTT | pYES8D | 61 |
| | GTC | | |
| CH353 | AAAAAATGTAGAGGTCGAGTTTAG | pYES8D, | 62 |
| | | pYES10 | |
| CH361 | TAAAAGACTCTAACAAAATAGC | Vio-1 | 63 |
| CH362 | ATGGGTTACGATGCTTTGTTC | Vio-2 | 64 |
| CH363 | TAATTTGTTCCAAGTAACCATC | Vio-2 | 65 |
| CH364 | TTGGAGAGGTTGTGTTGCTG | Vio-3 | 66 |
| CH365 | CCACAAAGCCAATCTAGCAC | Vio-3 | 67 |
| CH366 | GTGAAGCTGATAGAGGTGATG | Vio-4 | 68 |
| CH367 | TCATCTCTCAACAACAAATC | Vio-4 | 69 |
| CH368 | CCAGATTTGGGTGCTAAATTG | Vio-5 | 70 |
| CH369 | TCTCTTCTTCTATCACCAGAAC | Vio-5 | 71 |
| CH370 | ACTGCTGAACAATTACAATTG | Vio-6 | 72 |
| CH371 | GGTTCCAACATGGAGGCTTG | Vio-6 | 73 |
| CH372 | TTGGCTAGAAGATGTGAAAG | Vio-7 | 74 |
| CH373 | AAGGCTCTCATAGTAGGTTC | Vio-7 | 75 |
| CH374 | TCTGGTTCTCCATCTTTGAC | Vio-8 | 76 |
| CH375 | CTTTCGAATCTGATGGCAATAC | Vio-8 | 77 |
| CH376 | GCTTTGAGAGATAAGTGTAG | Vio-9 | 78 |
| CH377 | CAGTAACCAGAAGTCAATTG | Vio-9 | 79 |
| CH396 | GAGTAAACTTGGTCTGACAGTCAGAAGAACTCGTC | pYES10 | 80 |
| | AAGAAG | | |
| CH397 | CTTCTTGACGAGTTCTTCTGACTGTCAGACCAAGTT | pYES10 | 81 |
| | TACTC | | |
| CH399 | GAAAAGTGCCACCTGACGT | pYES10 | 82 |
| CH428 | TCAAGAAGGCGATAGAAGG | pASE101 | 83 |
| CH438 | TTTTTTCTGCGCGTAATCTG | pASE101 | 84 |
| CH476 | CTTGAGATCCTTTTTTTCTGCGCGTAATCTGC | pASE_cont | 85 |
| CH477 | TCAGAAGAACTCGTCAAGAAGGCGATAGAAGGCGA | pASE_cont | 86 |
| | TG | | |
| CH400 | TGGTTTCTTAGACGTCAGGTGGCACTTTTCAACCGG | pYES10 | 87 |
| | AATTGCCAGCTG | | |
| CH401 | CTAAACTCGACCTCTACATTTTTTGAAATTTGCTATT | pYES10 | 88 |
| | TTGTTAGAGTCTTTTACACCATTTGTC | | |
| CH450 | GCCTTCTATCGCCTTCTTTGAGCTCATACACCCAAA | pASE101 | 89 |
| | CAG | | |
| CH451 | AGATTACGCGCAGAAAAAACAAGAATTCTTACTAC | pASE101 | 90 |
| | GCAC | | |
| CH452 | GTAAAAGACTCTAACAAAATAGCAAATTTCGTCAA | PilAD-1 | 91 |
| | AAATGCTAAGAAATAGACTTTAGCCTTGAGATGAT | | |
| | G | | |
| CH453 | TCGGGCACCGAACTCCCCGAAG | PilAD-1 | 92 |
| CH454 | GTTAGCGCTTCGGGGAGTTC | PilAD-2 | 93 |
| CH455 | CGGCTGCACTTGCACTTGG | PilAD-2 | 94 |
| CH456 | TCTGGGATTAACCAAGTGCAAG | PilAD-3 | 95 |
| CH457 | TGCGCATCGCCTTGGAAAGTG | PilAD-3 | 96 |
| CH458 | CGGGCACGCCACTTTCCAAG | PilAD-4 | 97 |
| CH459 | TAGATCACCACATTGAGAAAG | PilAD-4 | 98 |
| CH460 | TGTCGGTAGCTTTCTCAATGTG | PilAD-5 | 99 |
| CH461 | CCGATTGGTATCACGCACGTCACGTGCGATCACATC | PilAD-5 | 100 |
| | GGCATCGAC | | |
| CH462 | ATCGCACGTGACGTGCGTGATACCAATCGGGTCAA | PilMQ-1 | 101 |
| | AACCGTAGTG | | |
| CH463 | TGGACGTCGATACGCACGGCTAG | PilMQ-1 | 102 |
| CH464 | CAACTCGCTAGCCGTGCGTATC | PilMQ-2 | 103 |
| CH465 | TGGGGTTAAAAATTGGAAGGAG | PilMQ-2 | 104 |
| CH466 | TTTAAAGTCTCCTTCCAATTTTTAAC | PilMQ-3 | 105 |
| CH467 | GCCTTAACCTTGACCACACTC | PilMQ-3 | 106 |
| CH468 | GCCGGCAGGGAGTGTGGTCAAG | PilMQ-4 | 107 |
| CH469 | TCAGACAACATGTTCACGTTG | PilMQ-4 | 108 |
| CH470 | CGGCGGTGAAGGCAACGTGAAC | PilMQ-5 | 109 |
| CH471 | TTGCATCTAAACTCGACCTCTACATTTTTTATGTTTA | PilMQ-5 | 110 |
| | TCTTTCCTCACCGATATTTCGTG | | |
| CH472 | CAGATTACGCGCAGAAAAAAAGGATCTCAAGACTT | PilAD-1 | 111 |
| | TAGCCTTGAGATGATG | (E.coli) | |
| CH473 | GCCTTCTATCGCCTTCTTGACGAGTTCTTCTGATTCC | PilMQ-5 | 112 |
| | TCACCGATATTTCGTG | (E.coli) | |
| CH478 | CAGATTACGCGCAGAAAAAAAGGATCTCAAGCTAA | VioAE-1 | 113 |
| | ATTGTAAGCGTTAATATTTTG | | |
| CH479 | CAGGCTAAAACGCGCACCTG | VioAE-1 | 114 |
| CH480 | CAGGTGCGCGTTTTAGCCTG | VioAE-2 | 115 |
| CH481 | CAGACCGTCACCACGATCCG | VioAE-2 | 116 |
| CH482 | CGGATCGTGGTGACGGTCTG | VioAE-3 | 117 |
| CH483 | CTCGATACGATGCGGGATATC | VioAE-3 | 118 |
| CH484 | GATATCCCGCATCGTATCGAG | VioAE-4 | 119 |
| CH485 | CACGGTTGGTCAGCTCATTC | VioAE-4 | 120 |
| CH486 | GAATGAGCTGACCAACCGTG | VioAE-5 | 121 |
| CH487 | CAGCGGACGGAAATCCTCC | VioAE-5 | 122 |
| CH488 | GGAGGATTTCCGTCCGCTG | VioAE-6 | 123 |
| CH489 | TTACCTCCTTAAAGATCTTC | VioAE-6 | 124 |
| CH490 | GAAGATCTTTAAGGAGGTAA | VioAE-7 | 125 |
| CH491 | CGACGGTTTCGAACCAAAC | VioAE-7 | 126 |
| CH492 | GTTTGGTTCGAAACCGTCG | VioAE-8 | 127 |
| CH493 | GCCTTCTATCGCCTTCTTGACGAGTTCTTCTGATTAG | VioAE-8 | 128 |
| | CGCTTGGCCGCGAAAAC | | |
| CHPT1 | TCA GAA GAA CTC GTC FFG AAG GCG | pASE_cont | 129 |
| | | (PT) | |
| CHPT2 | CTT GAG ATC CTT TTT ZZC TGC GCG | pASE_cont | 130 |
| | | (PT) | |
| CHPT3 | TCA AGA AGG CGA TAG FFG GCG ATG | pASE101L | 131 |
| | | (PT) | |
| CHPT4 | TTT TTT CTG CGC GTA FZC TGC TGC TTG C | pASE101L | 132 |
| | | (PT) | |
| CHPT5 | TACGCGCAGAAAAAAFEGATCTCAAGACTTTAGCCT | PilAD-1 (PT) | 133 |
| | TGAG | | |
| CHPT6 | TCGGGCACCGAACTCOOCGAAGCGCTAAC | PilAD-1 (PT) | 134 |
| CHPT7 | GAGTTCGGTGCCCGAEECGCTGCTTGAG | PilAD-2 (PT) | 135 |
| CHPT8 | CGGCTGCACTTGCACZZGGTTAATCCCAG | PilAD-2 (PT) | 136 |
| CHPT9 | GTGCAAGTGCAGCCGFFAATCGGCTTTGGCTTTG | PilAD-3 (PT) | 137 |
| CHPT10 | TGCGCATCGCCTTGGFFAGTGGCGTGCCCG | PilAD-3 (PT) | 138 |
| CHPT11 | CCAAGGCGATGCGCAOOGCCAGCGCCCATTTTG | PilAD-4 (PT) | 139 |
| CHPT12 | TAGATCACCACATTGFEAAAGCTACCGAC | PilAD-4 (PT) | 140 |
| CHPT13 | CAATGTGGTGATCTAZOGCTTACCCAAAATCATG | PilAD-5 (PT) | 141 |
| CHPT14 | CCGATTGGTATCACGOFCGTCACGTGCGATCAC | PilAD-5 (PT) | 142 |
| CHPT15 | CGTGATACCAATCGGEZCAAAACCGTAGTG | PilMQ-1 (PT) | 143 |
| CHPT16 | TGGACGTCGATACGCFOGGCTAGCGAGTTG | PilMQ-1 (PT) | 144 |
| CHPT17 | GCGTATCGACGTCCAEFCTGGATGTTGGTGGATATT | PilMQ-2 (PT) | 145 |
| | G | | |
| CHPT18 | TGGGGTTAAAAATTGEFAGGAGACTTTAAAG | PilMQ-2 (PT) | 146 |
| CHPT19 | CAATTTTTAACCCCAEOCTCTAACCCGCAAGAG | PilMQ-3 (PT) | 147 |
| CHPT20 | GCCTTAACCTTGACCFOACTCCCTGCCGGCGTTTG | PilMQ-3 (PT) | 148 |
| CHPT21 | GGTCAAGGTTAAGGCEEGTCAATATGTCGGAATC | PilMQ-4 (PT) | 149 |
| CHPT22 | TCAGACAACATGTTCFOGTTGCCTTCACCGCCGATC | PilMQ-4 (PT) | 150 |
| CHPT23 | GAACATGTTGTCTGAFOGAGGTTCGATCAGCATC | PilMQ-5 (PT) | 151 |
| CHPT24 | CTATCGCCTTCTTGAOEAGTTCTTCTGATTC | PilAD-1 (PT) | 152 |

PT, phosphorothioate; F, PT-deoxyadenine; O, PT-deoxycytosine; E, PT-deoxyguanidine; Z, PT-deoxythymidine

TABLE 9
DNA fragments for assembly test in this study.

| DNA Fragment | Host | Size | DNA type | Primer set | Source |
|---|---|---|---|---|---|
| Vio-1 | Yeast | 600 bp | Synthetic | NA | C. violaceum |
| Vio-2 | Yeast | 600 bp | Synthetic | NA | C. violaceum |
| Vio-3 | Yeast | 750 bp | Synthetic | NA | C. violaceum |
| Vio-4 | Yeasti | 750 bp | Synthetic | NA | C. violaceum |
| Vio-5 | Yeast | 999 bp | Synthetic | NA | C. violaceum |
| Vio-6 | Yeast | 999 bp | Synthetic | NA | C. violaceum |
| Vio-7 | Yeast | 999 bp | Synthetic | NA | C. violaceum |
| Vio-8 | Yeast | 999 bp | Synthetic | NA | C. violaceum |
| Vio-9 | Yeast | 999 bp | Synthetic | NA | C. violaceum |
| Vio-10 | Yeast | 588 bp | Synthetic | NA | C. violaceum |
| Vio-1 | Yeast | 589 bp | Pre-cloned | NA | C. violaceum |
| (BamHI/XhoI) | | | | | |
| Vio-2 | Yeast | 589 bp | Pre-cloned | NA | C. violaceum |
| (BamHI/XhoI) | | | | | |
| Vio-3 | Yeast | 739 bp | Pre-cloned | NA | C. violaceum |
| (BamHI/XhoI) | | | | | |
| Vio-4 | Yeasti | 988 bp | Pre-cloned | NA | C. violaceum |
| (BamHI/XhoI) | | | | | |
| Vio-5 | Yeast | 989 bp | Pre-cloned | NA | C. violaceum |
| (BamHI/XhoI) | | | | | |
| Vio-6 | Yeast | 989 bp | Pre-cloned | NA | C. violaceum |
| (BamHI/XhoI) | | | | | |
| Vio-7 | Yeast | 989 bp | Pre-cloned | NA | C. violaceum |
| (BamHI/XhoI) | | | | | |
| Vio-8 | Yeast | 989 bp | Pre-cloned | NA | C. violaceum |
| (BamHI/XhoI) | | | | | |
| Vio-9 | Yeast | 989 bp | Pre-cloned | NA | C. violaceum |
| (BamHI/XhoI) | | | | | |
| Vio-10 | Yeast | 577 bp | Pre-cloned | NA | C. violaceum |
| (BamHI/XhoI) | | | | | |
| Vio-1 | Yeast | 580 bp | PCR | CH312 & CH361 | C. violaceum |
| | | | amplified | | |
| Vio-2 | Yeast | 680 bp | PCR | CH362 & CH363 | C. violaceum |
| | | | amplified | | |
| Vio-3 | Yeast | 730 bp | PCR | CH364 & CH365 | C. violaceum |
| | | | amplified | | |
| Vio-4 | Yeast | 730 bp | PCR | CH366 & CH367 | C. violaceum |
| | | | amplified | | |
| Vio-5 | Yeast | 980 bp | PCR | CH368 & CH369 | C. violaceum |
| | | | amplified | | |
| Vio-6 | Yeast | 980 bp | PCR | CH370 & CH371 | C. violaceum |
| | | | amplified | | |
| Vio-7 | Yeast | 980 bp | PCR | CH372 & CH373 | C. violaceum |
| | | | amplified | | |
| Vio-8 | Yeast | 980 bp | PCR | CH374 & CH375 | C. violaceum |
| | | | amplified | | |
| Vio-9 | Yeast | 980 bp | PCR | CH376 & CH377 | C. violaceum |
| | | | amplified | | |
| Vio-10 | Yeast | 568 bp | PCR | CH313 & CH357 | C. violaceum |
| | | | amplified | | |
| PilAD-1 | Yeast | 1051 bp | PCR | CH452& CH453 | V. cholera |
| | | | amplified | | |
| PilAD-2 | Yeast/E. coli | 1029 bp | PCR | CH454 & CH455 | V. cholerae |
| | | | amplified | | |
| PilAD-3 | Yeast/E. coli | 1030 bp | PCR | CH456 & CH457 | V. cholerae |
| | | | amplified | | |
| PilAD-4 | Yeast/E. coli | 1030 bp | PCR | CH458 & CH459 | V. cholerae |
| | | | amplified | | |
| PilAD-5 | Yeast/E. coli | 945 bp | PCR | CH460 & CH461 | V. cholerae |
| | | | amplified | | |
| PilMQ-1 | Yeast/E. coli | 1015 bp | PCR | CH462 & CH463 | V. cholerae |
| | | | amplified | | |
| PilMQ-2 | Yeast/E. coli | 1030 bp | PCR | CH464 & CH465 | V. cholerae |
| | | | amplified | | |
| PilMQ-3 | Yeast/E. coli | 1030 bp | PCR | CH466 & CH467 | V. cholerae |
| | | | amplified | | |
| PilMQ-4 | Yeast/E. coli | 1030 bp | PCR | CH468 & CH469 | V. cholerae |
| | | | amplified | | |
| PilMQ-5 | Yeast | 1041 bp | PCR | CH470 & CH471 | V. cholerae |
| | | | amplified | | |
| PilAD-1 | E. coli | 1026 bp | PCR | CH472 & CH453 | V. cholerae |
| (normal) | | | amplified | | |
| PilMQ-5 | E. coli | 1011 bp | PCR | CH470 & CH473 | V. cholerae |
| (normal) | | | amplified | | |
| PilAD-1 (PT) | E. coli | 1026 bp | PCR | CHPT5 & | V. cholerae |
| | | | amplified | CHPT6 | |
| PilAD-2 (PT) | E. coli | 1015 bp | PCR | CHPT7 & | V. cholerae |
| | | | amplified | CHPT8 | |
| PilAD-3 (PT) | E. coli | 1015 bp | PCR | CHPT9 & | V. cholerae |
| | | | amplified | CHPT10 | |
| PilAD-4 (PT) | E. coli | 1015 bp | PCR | CHPT11 & | V. cholerae |
| | | | amplified | CHPT12 | |
| PilAD-5 (PT) | E. coli | 930 bp | PCR | CHPT13 & | V. cholerae |
| | | | amplified | CHPT14 | |
| PilMQ-1 (PT) | E. coli | 1000 bp | PCR | CHPT15 & | V. cholerae |
| | | | amplified | CHPT16 | |
| PilMQ-2 (PT) | E. coli | 1015 bp | PCR | CHPT17 & | V. cholerae |
| | | | amplified | CHPT18 | |
| PilMQ-3 (PT) | E. coli | 1015 bp | PCR | CHPT19 & | V. cholerae |
| | | | amplified | CHPT20 | |
| PilMQ-4 (PT) | E. coli | 1015 bp | PCR | CHPT21 & | V. cholerae |
| | | | amplified | CHPT22 | |
| PilMQ-5 (PT) | E. coli | 1011 bp | PCR | CHPT23 & | V. cholera |
| | | | amplified | CHPT24 | |
| VioAE-1 | E. coli | 1031 bp | PCR | CH478 & CH479 | C. violaceum |
| | | | amplified | | |
| VioAE-2 | E. coli | 1021 bp | PCR | CH480 & CH481 | C. violaceum |
| | | | amplified | | |
| VioAE-3 | E. coli | 1013 bp | PCR | CH482 & CH483 | C. violaceum |
| | | | amplified | | |
| VioAE-4 | E. coli | 1027 bp | PCR | CH484 & CH485 | C. violaceum |
| | | | amplified | | |
| VioAE-5 | E. coli | 1020 bp | PCR | CH486 & CH487 | C. violaceum |
| | | | amplified | | |
| VioAE-6 | E. coli | 1009 bp | PCR | CH488 & CH489 | C. violaceum |
| | | | amplified | | |
| VioAE-7 | E. coli | 1028 bp | PCR | CH490 & CH491 | C. violaceum |
| | | | amplified | | |
| VioAE-8 | E. coli | 770 bp | PCR | CH492 & CH493 | C. violaceum |
| | | | amplified | | |
| VioAE-14 | E. coli | 4031 bp | PCR | CH478 & CH485 | C. violaceum |
| | | | amplified | | |
| VioAE-56 | E. coli | 2010 bp | PCR | CH486 & CH489 | C. violaceum |
| | | | amplified | | |
| VioAE-78 | E. coli | 1779 bp | PCR | CH490 & CH493 | C. violaceum |
| | | | amplified | | |
| VioAE-58 | E. coli | 3769 bp | PCR | CH486 & CH493 | C. violaceum |
| | | | amplified | | |

PT, phosphorothioate;
NA, not applicable

TABLE 10
pYES8D Sequence

AAAAAATGTAGAGGTCGAGTTTAGATGCAAGTTCAAGGAGCGAAAGGTGG
ATGGGTAGGTTATATAGGGATATAGCACAGAGATATATAGCAAAGAGATA
CTTTTGAGCAATGTTTGTGGAAGCGGTATTCGCAATGGGAAGCTCCACCC
CGGTTGATAATCAGAAAGCTTCAACCAAAGCTTCAGGGGATAACGCAGGA
AAGAACATGTGAGCAAAAGGCCAGCAAAAGCCCAGGAACCGTAAAAAGGC
CGCGTTGCTGGCGTTTTTCCATAGGCTCCGCCCCCCTGACGAGCATCACA
AAAATCGACGCTCAAGTCAGAGGTGGCGAAACCCGACAGGACTATAAAGA
TACCAGGCGTTTCCCCCTGGAAGCTCCCTCGTGCGCTCTCCTGTTCCGAC
CCTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGTT
CGCTCCAAGCTGGGCTGTGTGCACGAACCCCCCGTTCAGCCCGACCGCTG
CGCCTTATCCGGTAACTATCGTCTTGAGTCCAACCCGGTAAGACACGACT
TATCGCCACTGGCAGCAGCCACTGGTAACAGGATTAGCAGAGCGAGGTAT
GTAGGCGGTGCTACAGAGTTCTTGAAGTGGTGGCCTAACTACGGCTACAC
TAGAAGGACAGTATTTGGTATCTGCGCTCTGCTGAAGCCAGTTACCTTCG
GAAAAAGAGTTGGTAGCTCTTGATCCGGCAAACAAACCACCGCTGGTAGC
GGTGGTTTTTTTGTTTGCAAGCAGCAGATTACGCGCAGAAAAAAAGGATC
TCAAGAAGATCCTTTGATCTTTTCTACGGGGTCTGACGCTCAGTGGAACG
AAAACTCACGTTAAGGGATTTTGGTCATGAGATTATCAAAAAGGATCTTC
ACCTAGATCCTTTTAAATTAAAAATGAAGTTTTAAATCAATCTAAAGTAT
ATATGAGTAAACTTGGTCTGACAGTTACCAATGCTTAATCAGTGAGGCAC
CTATCTCAGCGATCTGTCTATTTCGTTCATCCATAGTTGCCTGACTCCCC
GTCGTGTAGATAACTACGATACGGGAGCGCTTACCATCTGGCCCCAGTGC
TGCAATGATACCGCGAGACCCACGCTCACCGGCTCCAGATTTATCAGCAA
TAAACCAGCCAGCCGGAAGGGCCGAGCGCAGAAGTGGTCCTGCAACTTTA
TCCGCCTCCATCCAGTCTATTAATTGTTGCCGGGAAGCTAGAGTAAGTAG
TTCGCCAGTTAATAGTTTGCGCAACGTTGTTGCCATTGCTACAGGCATCG
TGGTGTCACGCTCGTCGTTTGGTATGGCTTCATTCAGCTCCGGTTCCCAA
CGATCAAGGCGAGTTACATGATCCCCCATGTTGTGCAAAAAAGCGGTTAG
CTCCTTCGGTCCTCCGATCGTTGTCAGAAGTAAGTTGGCCGCAGTGTTAT
CACTCATGGTTATGGCAGCACTGCATAATTCTCTTACTGTCATGCCATCC
GTAAGATGCTTTTCTGTGACTGGTGAGTACTCAACCAAGTCATTCTGAGA
ATAGTGTATGCGGCGACCGAGTTGCTCTTGCCCGGCGTCAACACGGGATA
ATACCGCGCCACATAGCAGAACTTTAAAAGTGCTCATCATTGGAAAACGT
TCTTCGGGGCGAAAACTCTCAAGGATCTTACCGCTGTTGAGATCCAGTTC
GATGTAACCCACTCGTGCACCCAACTGATCTTCAGCATCTTTTACTTTCA
CCAGCGTTTCTGGGTGAGCAAAAACAGGAAGGCAAAATGCCGCAAAAAAG
GGAATAAGGGCGACACGGAAATGTTGAATACTCATACTCTTCCTTTTTCA
ATATTATTGAAGCATTTATCAGGGTTATTGTCTCATGAGCGGATACATAT
TTGAATGTATTTAGAAAAATAAACAAATAGGGGTTCCGCGCACATTTCCC
CGAAAAGTGCCACCTGACGTCTAAGAAACCATTATTATCATGACATTAAC
CTATAAAAATAGGCGTATCACGAGGCCCTTTCGTCTTCAAGAAATTCGGT
CGAAAAAAGAAAAGGAGAGGGCCAAGAGGGAGGGCATTGGTGACTATTGA
GCACGTGAGTATACGTGATTAAGCACACAAAGGCAGCTTGGAGTATGTCT
GTTATTAATTTCACAGGTAGTTCTGGTCCATTGGTGAAAGTTTGCGGCTT
GCAGAGCACAGAGGCCGCAGAATGTGCTCTAGATTCCGATGCTGACTTGC
TGGGTATTATATGTGTGCCCAATAGAAAGAGAACAATTGACCCGGTTATT
GCAAGGAAAATTTCAAGTCTTGTAAAAGCATATAAAAATAGTTCAGGCAC
TCCGAAATACTTGGTTGGCGTGTTTCGTAATCAACCTAAGGAGGATGTTT
TGGCTCTGGTCAATGATTACGGCATTGATATCGTCCAACTGCACGGAGAT
GAGTCGTGGCAAGAATACCAAGAGTTCCTCGGTTTGCCAGTTATTAAAAG
ACTCGTATTTCCAAAAGACTGCAACATACTACTCAGTGCAGCTTCACAGA
AACCTCATTCGTTTATTCCCTTGTTTGATTCAGAAGCAGGTGGGACAGGT
GAACTTTTGGATTGGAACTCGATTTCTGACTGGGTTGGAAGGCAAGAGAG
CCCCGAGAGCTTACATTTTATGTTAGCTGGTGGACTGACGCCAGAAAATG
TTGGTGATGCGCTTAGATTAAATGGCGTTATTGGTGTTGATGTAAGCGGA
GGTGTGGAGACAAATGGTGTAAAAGACTCTAACAAAATAGCAAATTTC
(SEQ ID NO: 153)

TABLE 11
pYES8 Sequence

TATTTAAGTATTGTTTGTGCACTTGCCCTAGCTTATCGATGATAAGCTGT
CAAAGATGAGAATTAATTCCACGGACTATAGACTATACTAGATACTCCGT
CTACTGTACGATACACTTCCGCTCAGGTCCTTGTCCTTTAACGAGGCCTT
ACCACTCTTTTGTTACTCTATTGATCCAGCTCAGCAAAGGCAGTGTGATC
TAAGATTCTATCTTCGCGATGTAGTAAAACTAGCTAGACCGAGAAAGAGA
CTAGAAATGCAAAAGGCACTTCTACAATGGCTGCCATCATTATTATCCGA
TGTGACGCTGCAGCTTCTCAATGATATTCGAATACGCTTTGAGGAGATAC
AGCCTAATATCCGACAAACTGTTTTACAGATTTACGATCGTACTTGTTAC
CCATCATTGAATTTTGAACATCCGAACCTGGGAGTTTTCCCTGAAACAGA
TAGTATATTTGAACCTGTATAATAATATATAGTCTAGCGCTTTACGGAAG
ACAATGTATGTATTTCGGTTCCTGGAGAAACTATTGCATCTATTGCATAG
GTAATCTTGCACGTCGCATCCCCGGTTCATTTTCTGCGTTTCCATCTTGC
ACTTCAATAGCATATCTTTGTTAACGAAGCATCTGTGCTTCATTTTGTAG
AACAAAAATGCAACGCGAGAGCGCTAATTTTTCAAACAAAGAATCTGAGC
TGCATTTTTACAGAACAGAAATGCAACGCGAAAGCGCTATTTTACCAACG
AAGAATCTGTGCTTCATTTTTGTAAAACAAAAATGCAACGCGACGAGAGC
GCTAATTTTTCAAACAAAGAATCTGAGCTGCATTTTTACAGAACAGAAAT
GCAACGCGAGAGCGCTATTTTACCAACAAAGAATCTATACTTCTTTTTTG
TTCTACAAAAATGCATCCCGAGAGCGCTATTTTTCTAACAAAGCATCTTA
GATTACTTTTTTTCTCCTTTGTGCGCTCTATAATGCAGTCTCTTGATAAC
TTTTTGCACTGTAGGTCCGTTAAGGTTAGAAGAAGGCTACTTTGGTGTCT
ATTTTCTCTTCCATAAAAAAAGCCTGACTCCACTTCCCGCGTTTACTGAT
TACTAGCGAAGCTGCGGGTGCATTTTTTCAAGATAAAGGCATCCCCGATT
ATATTCTATACCGATGTGGATTGCGCATACTTTGTGAACAGAAAGTGATA
GCGTTGATGATTCTTCATTGGTCAGAAAATTATGAACGGTTTCTTCTATT
TTGTCTCTATATACTACGTATAGGAAATGTTTACATTTTCGTATTGTTTT
CGATTCACTCTATGAATAGTTCTTACTACAATTTTTTTGTCTAAAGAGTA
ATACTAGAGATAAACATAAAAAATGTAGAGGTCGAGTTTAGATGCAAGTT
CAAGGAGCGAAAGGTGGATGGGTAGGTTATATAGGGATATAGCACAGAGA
TATATAGCAAAGAGATACTTTTGAGCAATGTTTGTGGAAGCGGTATTCGC
AATGGGAAGCTCCACCCCGGTTGATAATCAGAAAGCTTCAACCAAAGCTT
CAGGGGATAACGCAGGAAAGAACATGTGAGCAAAAGGCCAGCAAAAGCCC
AGGAACCGTAAAAAGGCCGCGTTGCTGGCGTTTTTCCATAGGCTCCGCCC
CCCTGACGAGCATCACAAAAATCGACGCTCAAGTCAGAGGTGGCGAAACC
CGACAGGACTATAAAGATACCAGGCGTTTCCCCCTGGAAGCTCCCTCGTG
CGCTCTCCTGTTCCGACCCTGCCGCTTACCGGATACCTGTCCGCCTTTCT
CCCTTCGGGAAGCGTGGCGCTTTCTCATAGCTCACGCTGTAGGTATCTCA
GTTCGGTGTAGGTCGTTCGCTCCAAGCTGGGCTGTGTGCACGAACCCCCC
GTTCAGCCCGACCGCTGCGCCTTATCCGGTAACTATCGTCTTGAGTCCAA
CCCGGTAAGACACGACTTATCGCCACTGGCAGCAGCCACTGGTAACAGGA
TTAGCAGAGCGAGGTATGTAGGCGGTGCTACAGAGTTCTTGAAGTGGTGG
CCTAACTACGGCTACACTAGAAGGACAGTATTTGGTATCTGCGCTCTGCT
GAAGCCAGTTACCTTCGGAAAAAGAGTTGGTAGCTCTTGATCCGGCAAAC
AAACCACCGCTGGTAGCGGTGGTTTTTTTGTTTGCAAGCAGCAGATTACG
CGCAGAAAAAAAGGATCTCAAGAAGATCCTTTGATCTTTTCTACGGGGTC
TGACGCTCAGTGGAACGAAAACTCACGTTAAGGGATTTTGGTCATGAGAT
TATCAAAAAGGATCTTCACCTAGATCCTTTTAAATTAAAAATGAAGTTTT
AAATCAATCTAAAGTATATATGAGTAAACTTGGTCTGACAGTTACCAATG
CTTAATCAGTGAGGCACCTATCTCAGCGATCTGTCTATTTCGTTCATCCA
TAGTTGCCTGACTCCCCGTCGTGTAGATAACTACGATACGGGAGCGCTTA
CCATCTGGCCCCAGTGCTGCAATGATACCGCGAGACCCACGCTCACCGGC
TCCAGATTTATCAGCAATAAACCAGCCAGCCGGAAGGGCCGAGCGCAGAA
GTGGTCCTGCAACTTTATCCGCCTCCATCCAGTCTATTAATTGTTGCCGG
GAAGCTAGAGTAAGTAGTTCGCCAGTTAATAGTTTGCGCAACGTTGTTGC
CATTGCTACAGGCATCGTGGTGTCACGCTCGTCGTTTGGTATGGCTTCAT
TCAGCTCCGGTTCCCAACGATCAAGGCGAGTTACATGATCCCCCATGTTG
TGCAAAAAAGCGGTTAGCTCCTTCGGTCCTCCGATCGTTGTCAGAAGTAA
GTTGGCCGCAGTGTTATCACTCATGGTTATGGCAGCACTGCATAATTCTC
TTACTGTCATGCCATCCGTAAGATGCTTTTCTGTGACTGGTGAGTACTCA
ACCAAGTCATTCTGAGAATAGTGTATGCGGCGACCGAGTTGCTCTTGCCC
GGCGTCAACACGGGATAATACCGCGCCACATAGCAGAACTTTAAAAGTGC
TCATCATTGGAAAACGTTCTTCGGGGCGAAAACTCTCAAGGATCTTACCG
CTGTTGAGATCCAGTTCGATGTAACCCACTCGTGCACCCAACTGATCTTC
AGCATCTTTTACTTTCACCAGCGTTTCTGGGTGAGCAAAAACAGGAAGGC
AAAATGCCGCAAAAAAGGGAATAAGGGCGACACGGAAATGTTGAATACTC
ATACTCTTCCTTTTTCAATATTATTGAAGCATTTATCAGGGTTATTGTCT
CATGAGCGGATACATATTTGAATGTATTTAGAAAAATAAACAAATAGGGG
TTCCGCGCACATTTCCCCGAAAAGTGCCACCTGACGTCTAAGAAACCATT
ATTATCATGACATTAACCTATAAAAATAGGCGTATCACGAGGCCCTTTCG
TCTTCAAGAAATTCGGTCGAAAAAAGAAAAGGAGAGGGCCAAGAGGGAGG
GCATTGGTGACTATTGAGCACGTGAGTATACGTGATTAAGCACACAAAGG
CAGCTTGGAGTATGTCTGTTATTAATTTCACAGGTAGTTCTGGTCCATTG
GTGAAAGTTTGCGGCTTGCAGAGCACAGAGGCCGCAGAATGTGCTCTAGA
TTCCGATGCTGACTTGCTGGGTATTATATGTGTGCCCAATAGAAAGAGAA
CAATTGACCCGGTTATTGCAAGGAAAATTTCAAGTCTTGTAAAAGCATAT
AAAAATAGTTCAGGCACTCCGAAATACTTGGTTGGCGTGTTTCGTAATCA
ACCTAAGGAGGATGTTTTGGCTCTGGTCAATGATTACGGCATTGATATCG
TCCAACTGCACGGAGATGAGTCGTGGCAAGAATACCAAGAGTTCCTCGGT
TTGCCAGTTATTAAAAGACTCGTATTTCCAAAAGACTGCAACATACTACT
CAGTGCAGCTTCACAGAAACCTCATTCGTTTATTCCCTTGTTTGATTCAG
AAGCAGGTGGGACAGGTGAACTTTTGGATTGGAACTCGATTTCTGACTGG
GTTGGAAGGCAAGAGAGCCCCGAGAGCTTACATTTTATGTTAGCTGGTGG
ACTGACGCCAGAAAATGTTGGTGATGCGCTTAGATTAAATGGCGTTATTG
GTGTTGATGTAAGCGGAGGTGTGGAGACAAATGGTGTAAAAGACTCTAAC
AAAATAGCAAATTTCGTCAAAAATGCTAAGAAATAGGTTATTACTGAGTA
GTATT (SEQ ID NO: 154)

TABLE 12
pASE101 Sequence

ATGTGAGCAAAAGGCCAGCAAAAGCCCAGGAACCGTAAAAAGGCCGCGTT
GCTGGCGTTTTTCCATAGGCTCCGCCCCCCTGACGAGCATCACAAAAATC
GACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCC
GCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCC
AAGCTGGGCTGTGTGCACGAACCCCCCGTTCAGCCCGACCGCTGCGCCTT
ATCCGGTAACTATCGTCTTGAGTCCAACCCGGTAAGACACGACTTATCGC
CACTGGCAGCAGCCACTGGTAACAGGATTAGCAGAGCGAGGTATGTAGGC
GGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAA
GAGTTGGTAGCTCTTGATCCGGCAAACAAACCACCGCTGGTAGCGGTGGT
TTTTTTGTTTGCAAGCAGCAGATTACGCGCAGAAAAAACAAGAATTCTTA
CTACGCACCACCCTGCCACTCGTCGCAATACTGTTGCAGTTCATTCAGCA
TACGACCAACGTGGAAACCATCGCAAACCGCGTGGTGAACCTGGATCGCC
AGCGGCATCAGAACTTTGTCACCCTGGGTGTAGTATTTACCCATGGTGAA
GACCGGTGCGAAAAAGTTGTCCATGTTCGCTACATTCAGGTCGAAGCTAG
TGAAGGATACCCAAGGGTTCGCAGATACGAAGAACATATTTTCGATGAAG
CCTTTTGGGAAATACGCGAGATTCTCACCGTAACACGCAACGTCCTGAGA
GTAGATGTGCAGGAACTGACGGAAGTCGTCGTGGTATTCGCTCCACAGGC
TAGAGAAGGTTTCGGTCTGTTCGTGAAAAACGGTGTAGCACGGGTGAACA
GAGTCCCAGATAACCAGTTCGCCGTCTTTCATCGCCATACGAAATTCCGG
ATGGGCGTTCATGAGACGCGCCAGGATGTGGATGAAGGCCGGGTAGAATT
TGTGCTTGTTTTTCTTGACAGTCTTGAGGAATGCGGTGATGTCGAGCTGA
ACAGTTTGGTTGTAGGTGCACTGCGCAACGGACTGGAACGCTTCAAAGTG
CTCTTTACGATGCCACTGAGAGATGTCAACGGTCGTGTAGCCGGTGATCT
TTTTTTCCATTTTAGCTTCCTTAGCTCCTGAAAATCTCGATAACTCAAAA
AATACGCCCTCATACTAGATATCTAGATCCGGCCCGATGCGTCCGGCGTA
GAGGATCTGAAGATCAGCAGTTCAACCTGTTGATAGTACGTACTAAGCTC
TCATGTTTCACGTACTAAGCTCTCATGTTTAACGTACTAAGCTCTCATGT
TTAACGAACTAAACCCTCATGGCTAACGTACTAAGCTCTCATGGCTAACG
TACTAAGCTCTCATGTTTCACGTACTAAGCTCTCATGTTTGAACAATAAA
ATTAATATAAATCAGCAACTTAAATAGCCTCTAAGGTTTTAAGTTTTATA
AGAAAAAAAAGAATATATAAGGCTTTTAAAGCTTTTAAGGTTTAACGGTT
GTGGACAACAAGCCAGGGATGTAACGCACTGAGAAGCCCTTAGAGCCTCT
CAAAGCAATTTTCAGTGACACAGGAACACTTAACGGCTGACATGGGAATT
CTACTGTTTGGGTGTATGAGCTCAAAGAAGGCGATAGAAGGCGATGCGCT
GCGAATCGGGAGCGGCGATACCGTAAAGCACGAGGAAGCGGTCAGCCCAT
TCGCCGCCAAGCTCTTCAGCAATATCACGGGTAGCCAACGCTATGTCCTG
ATAGCGGTCCGCCACACCCAGCCGGCCACAGTCGATGAATCCAGAAAAGC
GGCCATTTTCCACCATGATATTCGGCAAGCAGGCATCGCCATGGGTCACG
ACGAGATCCTCGCCGTCGGGCATGCTCGCCTTGAGCCTGGCGAACAGTTC
GGCTGGCGCGAGCCCCTGATGCTCTTCGTCCAGATCATCCTGATCGACAA
GACCGGCTTCCATCCGAGTACGTGCTCGCTCGATGCGATGTTTCGCTTGG
TGGTCGAATGGGCAGGTAGCCGGATCAAGCGTATGCAGCCGCCGCATTGC
ATCAGCCATGATGGATACTTTCTCGGCAGGAGCAAGGTGAGATGACAGGA
GATCCTGCCCCGGCACTTCGCCCAATAGCAGCCAGTCCCTTCCCGCTTCA
GTGACAACGTCGAGCACAGCTGCGCAAGGAACGCCCGTCGTGGCCAGCCA
CGATAGCCGCGCTGCCTCGTCTTGCAGTTCATTCAGGGCACCGGACAGGT
CGGTCTTGACAAAAAGAACCGGGCGCCCCTGCGCTGACAGCCGGAACACG
GCGGCATCAGAGCAGCCGATTGTCTGTTGTGCCCAGTCATAGCCGAATAG
CCTCTCCACCCAAGCGGCCGGAGAACCTGCGTGCAATCCATCTTGTTCAA
TCATGCGAAACGATCCTCATCCTGTCTCTTGATCAGAGCTTGATCCCCTG
CGCCATCAGATCCTTGGCGGCGAGAAAGCCATCCAGTTTACTTTGCAGGG
CTTCCCAACCTTACCAGAGGGCGCCCCAGCTGGCAATTCCGGTTGAAAAG
TGCCACCTGACGTC (SEQ ID NO: 155)

TABLE 13
pASE_Cont Sequence

TCAGAAGAACTCGTCAAGAAGGCGATAGAAGGCGATGCGCTGCGAATCGG
GAGCGGCGATACCGTAAAGCACGAGGAAGCGGTCAGCCCATTCGCCGCCA
AGCTCTTCAGCAATATCACGGGTAGCCAACGCTATGTCCTGATAGCGGTC
CGCCACACCCAGCCGGCCACAGTCGATGAATCCAGAAAAGCGGCCATTTT
CCACCATGATATTCGGCAAGCAGGCATCGCCATGGGTCACGACGAGATCC
TCGCCGTCGGGCATGCTCGCCTTGAGCCTGGCGAACAGTTCGGCTGGCGC
GAGCCCCTGATGCTCTTCGTCCAGATCATCCTGATCGACAAGACCGGCTT
CCATCCGAGTACGTGCTCGCTCGATGCGATGTTTCGCTTGGTGGTCGAAT
GGGCAGGTAGCCGGATCAAGCGTATGCAGCCGCCGCATTGCATCAGCCAT
GATGGATACTTTCTCGGCAGGAGCAAGGTGAGATGACAGGAGATCCTGCC
CCGGCACTTCGCCCAATAGCAGCCAGTCCCTTCCCGCTTCAGTGACAACG
TCGAGCACAGCTGCGCAAGGAACGCCCGTCGTGGCCAGCCACGATAGCCG
CGCTGCCTCGTCTTGCAGTTCATTCAGGGCACCGGACAGGTCGGTCTTGA
CAAAAAGAACCGGGCGCCCCTGCGCTGACAGCCGGAACACGGCGGCATCA
GAGCAGCCGATTGTCTGTTGTGCCCAGTCATAGCCGAATAGCCTCTCCAC
CCAAGCGGCCGGAGAACCTGCGTGCAATCCATCTTGTTCAATCATGCGAA
ACGATCCTCATCCTGTCTCTTGATCAGAGCTTGATCCCCTGCGCCATCAG
ATCCTTGGCGGCGAGAAAGCCATCCAGTTTACTTTGCAGGGCTTCCCAAC
CTTACCAGAGGGCGCCCCAGCTGGCAATTCCGGTTGAAAAGTGCCACCTG
ACGTCATGTGAGCAAAAGGCCAGCAAAAGCCCAGGAACCGTAAAAAGGCC
GCGTTGCTGGCGTTTTTCCATAGGCTCCGCCCCCCTGACGAGCATCACAA
AAATCGACGCTCAAGTCAGAGGTGGCGAAACCCGACAGGACTATAAAGAT
ACCAGGCGTTTCCCCCTGGAAGCTCCCTCGTGCGCTCTCCTGTTCCGACC
CTGCCGCTTACCGGATACCTGTCCGCCTTTCTCCCTTCGGGAAGCGTGGC
GCTTTCTCATAGCTCACGCTGTAGGTATCTCAGTTCGGTGTAGGTCGTTC
GCTCCAAGCTGGGCTGTGTGCACGAACCCCCCGTTCAGCCCGACCGCTGC
GCCTTATCCGGTAACTATCGTCTTGAGTCCAACCCGGTAAGACACGACTT
ATCGCCACTGGCAGCAGCCACTGGTAACAGGATTAGCAGAGCGAGGTATG
TAGGCGGTGCTACAGAGTTCTTGAAGTGGTGGCCTAACTACGGCTACACT
AGAAGGACAGTATTTGGTATCTGCGCTCTGCTGAAGCCAGTTACCTTCGG
AAAAAGAGTTGGTAGCTCTTGATCCGGCAAACAAACCACCGCTGGTAGCG
GTGGTTTTTTTGTTTGCAAGCAGCAGATTACGCGCAGAAAAAAAGGATCT
CAAG (SEQ ID NO: 156)

### Example 3

### A Simple Method to Terminate GeneArt® Seamless Assembly Reaction Enable High Throughput Applications

The protocol below is directed, in part, to the termination of enzymatic reactions related to nucleic acid assembly. Once nucleic acid segments are fully assembled, the continued action of enzymes (e.g., exonucleases) can damage assembled nucleic acid molecules.

A linearized vector and DNA fragments is prepared as instructed in GeneArt® Seamless DNA assembly kit (Life Technologies, Catalog number A14606) manual. Add DNA mix in a volume of 10 μl to a thin-walled PCR tube or a well on a PCR plate. Add 10 μl of GeneArt Seamless DNA assembly enzyme mix, mix by pipetting up and down or flicking the tube. Brief spin down the liquid to the bottom of the tube (DO NOT exceed 5 seconds and 500 rpm). Incubate in a PCR machine with the following protocol if final construct is smaller than 13 kb: 30 minutes at 25° C., then 10 minutes at 75° C., hold at 4° C. If final the construct is larger than 13 kb, use the following protocol: 30 minutes at 25° C., 75 minutes at 75° C., then 60 minutes at 25° C., hold at 4° C. The reaction mixture can be stored at 25° C. or lower temperature for up to 48 hours until transformation.

TABLE 14

| Nucleotide sequence of pcDNA Rad51 BLM Exo1 Vector | Element |
|---|---|
| Fragment 1: | CMV |
| GTTAGGCGTTTTGCGCTGCTTCGCGATGTACGGGCCAGATATACG | Promoter |
| CGTTGACATTGATTATTGACTAGTTATTAATAGTAATCAATTACG | |
| GGGTCATTAGTTCATAGCCCATATATGGAGTTCCGCGTTACATAA | |
| CTTACGGTAAATGGCCCGCCTGGCTGACCGCCCAACGACCCCCG | |
| CCCATTGACGTCAATAATGACGTATGTTCCCATAGTAACGCCAAT | |
| AGGGACTTTCCATTGACGTCAATGGGTGGAGTATTTACGGTAAA | |
| CTGCCCACTTGGCAGTACATCAAGTGTATCATATGCCAAGTACGC | |
| CCCCTATTGACGTCAATGACGGTAAATGGCCCGCCTGGCATTATG | |
| CCCAGTACATGACCTTATGGGACTTTCCTACTTGGCAGTACATCT | |
| ACGTATTAGTCATCGCTATTACCATGGTGATGCGGTTTTGGCAGT | |
| ACATCAATGGGCGTGGATAGCGGTTTGACTCACGGGGATTTCCA | |
| AGTCTCCACCCCATTGACGTCAATGGGAGTTTGTTTTGGCACCAA | |
| AATCAACGGGACTTTCCAAAATGTCGTAACAACTCCGCCCCATT | |
| GACGCAAATGGGCGGTAGGCGTGTACGGTGGGAGGTCTATATAA | |
| GCAGAGCTCGTTTAGTGAACCGTCAGATCGCCTGGAGACGCCAT | |
| CCACGCTGTTTTGACCTCCATAGAAGACACCGGGACCGATCCAG | |
| CCTCCGGACTCTAGAGGATCGAATGGCAA (SEQ ID NO: 157) | |
| Fragment 2: | Rad51 |
| TGCAGATGCAGCTTGAAGCAAATGCAGATACTTCAGTGGAAGAA | |
| GAAAGCTTTGGCCCACAACCCATTTCACGGTTAGAGCAGTGTGG | |
| CATAAATGCCAACGATGTGAAGAAATTGGAAGAAGCTGGATTCC | |
| ATACTGTGGAGGCTGTTGCCTATGCGCCAAAGAAGGAGCTAATA | |
| AATATTAAGGGAATTAGTGAAGCCAAAGCTGATAAAATTCTGGC | |
| TGAGGCAGCTAAATTAGTTCCAATGGGTTTCACCACTGCAACTG | |
| AATTCCACCAAAGGCGGTCAGAGATCATACAGATTACTACTGGC | |
| TCCAAAGAGCTTGACAAACTACTTCAAGGTGGAATTGAGACTGG | |
| ATCTATCACAGAAATGTTTGGAGAATTCCGAACTGGGAAGACCC | |
| AGATCTGTCATACGCTAGCTGTCACCTGCCAGCTTCCCATTGACC | |
| GGGGTGGAGGTGAAGGAAAGGCCATGTACATTGACACTGAGGG | |
| TACCTTTAGGCCAGAACGGCTGCTGGCAGTGGCTGAGAGGTATG | |
| GTCTCTCTGGCAGTGATGTCCTGGATAATGTAGCATATGCTCGAG | |
| CGTTCAACACAGACCACCAGACCCAGCTCCTTTATCAAGCATCA | |
| GCCATGATGGTAGAATCTAGGTATGCACTGCTTATTGTAGACAGT | |
| GCCACCGCCCTTTACAGAACAGACTACTCGGGTCGAGGTGAGCT | |
| TTCAGCCAGGCAGATGCACTTGGCCAGGTTTCTGCGGATGCTTCT | |
| GCGACTCGCTGATGAGTTTGGTGTAGCAGTGGTAATCACTAATC | |
| AGGTGGTAGCTCAAGTGGATGGAGCAGCGATGTTTGCTGCTGAT | |
| CCCAAAAAACCTATTGGAGGAAATATCATCGCCCATGCATCAAC | |
| AACCAGATTGTATC (SEQ ID NO: 158) | |
| Fragment 3: | Rad51 |
| TGAGGAAAGGAAGAGGGGAAACCAGAATCTGCAAAATCTACGA | 2A Peptide |
| CTCTCCCTGTCTTCCTGAAGCTGAAGCTATGTTCGCCATTAATGC | BLM |
| AGATGGAGTGGGAGATGCCAAAGACGGAAGCGGAGCTACTAAC | |
| TTCAGCCTGCTGAAGCAGGCTGGAGACGTGGAGGAGAACCCTGG | |
| ACCTATGGCTGCTGTTCCTCAAAATAATCTACAGGAGCAACTAG | |
| AACGTCACTCAGCCAGAACACTTAATAATAAATTAAGTCTTTCA | |
| AAACCAAAATTTTCAGGTTTCACTTTTAAAAAGAAAACATCTTCA | |
| GATAACAATGTATCTGTAACTAATGTGTCAGTAGCAAAAACACC | |
| TGTATTAAGAAATAAAGATGTTAATGTTACCGAAGACTTTTCCTT | |
| CAGTGAACCTCTACCCAACACCACAAATCAGCAAAGGGTCAAGG | |
| ACTTCTTTAAAAATGCTCCAGCAGGACAGGAAACACAGAGAGGT | |
| GGATCAAAATCATTATTGCCAGATTTCTTGCAGACTCCGAAGGA | |
| AGTTGTATGCACTACCCAAAACACACCAACTGTAAAGAAATCCC | |
| GGGATACTGCTCTCAAGAAATTAGAATTTAGTTCTTCACCAGATT | |
| CTTTAAGTACCATCAATGATTGGGATGATATGGATGACTTTGATA | |
| CTTCTGAGACTTCAAAATCATTTGTTACACCACCCCAAAGTCACT | |
| TTGTAAGAGTAAGCACTGCTCAGAAATCAAAAAAGGGTAAGAG | |
| AAACTTTTTTAAAGCACAGCTTTATACAACAAACACAGTAAAGA | |
| CTGATTTGCCTCCACCCTCCTCTGAAAGCGAGCAAATAGATTTGA | |
| CTGAGGAACAGAAGGATGACTCAGAATGGTTAAGCAGCGATGTG | |
| ATTTGCATCGATGATGGCCCCATT (SEQ ID NO: 159) | |
| Fragment 4: | BLM |
| GCTGAAGTGCATATAAATGAAGATGCTCAGGAAAGTGACTCTCT | BLM |
| GAAAACTCATTTGGAAGATGAAAGAGATAATAGCGAAAAGAAG | |
| AAGAATTTGGAAGAAGCTGAATTACATTCAACTGAGAAAGTTCC | |
| ATGTATTGAATTTGATGATGATGATTATGATACGGATTTTGTTCC | |
| ACCTTCTCCAGAAGAAATTATTTCTGCTTCTTCTTCCTCTTCAAAA | |
| TGCCTTAGTACGTTAAAGGACCTTGACACATCTGACAGAAAAGA | |
| GGATGTTCTTAGCACATCAAAAGATCTTTTGTCAAAACCTGAGA | |
| AAATGAGTATGCAGGAGCTGAATCCAGAAACCAGCACAGACTGT | |
| GACGCTAGACAGATAAGTTTACAGCAGCAGCTTATTCATGTGAT | |
| GGAGCACATCTGTAAATTAATTGATACTATTCCTGATGATAAACT | |
| GAAACTTTTGGATTGTGGGAACGAACTGCTTCAGCAGCGGAACA | |
| TAAGAAGGAAACTTCTAACGGAAGTAGATTTTAATAAAAGTGAT | |
| GCCAGTCTTCTTGGCTCATTGTGGAGATACAGGCCTGATTCACTT | |
| GATGGCCCTATGGAGGGTGATTCCTGCCCTACAGGGAATTCTAT | |
| GAAGGAGTTAAATTTTTCACACCTTCCCTCAAATTCTGTTTCTCCT | |
| GGGGACTGTTTACTGACTACCACCCTAGGAAAGACAGGATTCTC | |
| TGCCACCAGGAAGAATCTTTTTGAAAGGCCTTTATTCAATACCCA | |
| TTTACAGAAGTCCTTTGTAAGTAGCAACTGGGCTGAAACACCAA | |
| GACTAGGAAAAAAAAATGAAAGCTCTTATTTCCCAGGAAATGTT | |
| CTCACAAGCACTGCTGTGAAAGATCAGAATAAACATACTGCTTC | |
| AATAAATGACTTAGAAAGAGAAACCCAACCTTCCTATGATATTG | |
| ATAATTTTGACATAGATGACTTTGATGATGATGATGACTGGGAA | |
| GACATAATGCATAATTTAGCAGCCAGCAAATCTTCCACAGCTGC | |
| CTATCAACCCATCAAGGAAGGTCGGCCAATTAAATCAGTATCAG | |
| AAAGACTTTCCTCAGCCAAGACAGACTGTCTTCCAGTGTCATCTA | |
| CTGCTCAAAATATAAACTTCTCAGAGTCAATTCAGAATTATACTG | |
| ACAAGTCAGCACAAAATTTAGCATCCAGAAATCTGAAACATGAG | |
| CGTTTCCAAAGTCTTAGTTTTCCTCATACAAAGGAAATGATGAAG | |
| ATTTTTCATAAAAAATTTGGCCTGCATAATTTTAGAACTAATCAG | |
| CTAGAGGCGATCAATGCTGCACTGCTTGGTGAAGACTGTTTTATC | |
| CTGATGCCGACTGGAGGTGGTAAGAGTTTGTGTTACCAGCTCCCT | |
| GCCTGTGTTTCTCCTGGGGTCACTGTTGTCATTTCTCCCTTGAGAT | |
| CACTTATCGTAGATCAAGTCCAAAAGCTGACTTCCTTGGATATTC | |
| CAGCTACATATCTGACAGGTGATAAGACTGACTCAGAAGCTACA | |
| AATATTTACCTCCAGTTATCAAAAAAAGACCCAATCATAAAACT | |
| TCTATATGTCACTCCAGAAAAGATCTGTGCAAGTAACAGACTCA | |
| TTTCTACTCTGGAGAATCTCTATGAGAGGAAGCTCTTGGCACGTT | |
| TTGTTATTGATGAAGCACATTGTGTCAGTCAGTGGGGACATGATT | |
| TTCGTCAAGATTACAAAAGAATGAATATGCTTCGCCAGAAGTTT | |
| CCTTCTGTTCCGGTGATGGCTCTTACGGCCACAGCTAATCCCAGG | |
| GTACAGAAGGACATCCTGACTCAGCTGAAGATTCTCAGACCTCA | |
| GGTGTTTAGCATGAGCTTTAACAGACATAATCTGAAATACTATGT | |
| ATTACCGAAAAAGCCTAAAAAGGTGGCATTTGATTGCCTAGAAT | |
| GGATCAGAAAGCACCACCCATATGATTCAGGGATAATTTACTGC | |
| CTCTCCAGGCGAGAATGTGACACCATGGCTGACACGTTACAGAG | |
| AGATGGGCTCGCTGCTCTTGCTTACCATGCTGGCCTCAGTGATTC | |
| TGCCAGAGATGAAGTGCAGCAGAAGTGGATTAATCAGGATGGCT | |
| GTCAGGTTATCTGTGCTACAATTGCATTTGGAATGGGGATTGACA | |
| AACCGGACGTGCGATTTGTGATTCATGCATCTCTCCCTAAATCTG | |
| TGGAGGGTTACTACCAAGAATCTGGCAGAGCTGGAAGAGATGGG | |
| GAAATATCTCACTGCCTGCTTTTCTATACCTATCATGATGTGACC | |
| AGACTGAAAAGACTTATAATGATGGAAAAAGATGGAAACCATC | |
| ATACAAGAGAAACTCACTTCAATAATTTGTATAGCATGGTACATT | |
| ACTGTGAAAATATAACGGAATGCAGGAGAATACAGCTTTTGGCC | |
| TACTTTGGTGAAAATGGATTTAATCCTGATTTTTGTAAGAAACAC | |
| CCAGATGTTTCTTGTGATAATTGCTGTAAAACAAAGGATTATAAA | |
| ACAAGAGATGTGACTGACGATGTGAAAAGTATTGTAAGATTTGT | |
| TCAAGAACATAGTTCATCACAAGGAATGAGAAATATAAAACATG | |
| TAGGTCCTTCTGGAAGATTTACTATGAATATGCTGGTCGACATTT | |
| TCTTGGGGAGTAAGAGTGCAAAAATCCAGTCAGGTATATTTGGA | |
| AAAGGATCTGCTTATTCACGACACAATGCCGAAAGACTTTTTAA | |
| AAAGCTGATACTTGACAAGATTTTGGATGAAGACTTATATATCA | |
| ATGCCAATGACCAGGCGATCGCTTATGTGATGCTCGGAAATAAA | |
| GCCCAAACTGTACTAAATGGCAATTTAAAGGTAGACTTTATGGA | |
| AACAGAAAATTCCAGCAGTGTGAAAAAACAAAAAGCGTTAGTA | |
| GCAAAAGTGTCTCAGAGGGAAGAGATGGTTAAAAAATGTCTTGG | |
| AGAACTTACAGAAGTCTGCAAATCTCTGGGGAAAGTTTTTGGTG | |
| TCCATTACTTCAATATTTTTAATACCGTCACTCTCAAGAAGCTTG | |
| CAGAATCTTTATCTTCTGATCCTGAGGTTTTGCTTCAAATTGATG | |
| GTGTTACTGAAGACAAACTGGAAAAATATGGTGCGGAAGTGATT | |
| TCAGTATTACAGAAATACTCTGAATGGACATCGCCAGCTGAAGA | |
| CAGTTCCCCAGGGATAAGCCTGTCCAGCAGCAGAGGCCCCGGAA | |
| GAAGTGCCGCTGAGGAGCTTGACGAGGAAATACCCGTATCTTCC | |
| CACTACTTTGCAAGTAAAACCAGAAATGAAAGGAAGAGGAAAA | |
| AGATGCCAGCCTCCCAAAGGTCTAAGAGGAGAAAAACTGCTTCC | |
| AGTGGTTCCAAGGCAAAGGGGGGGTCTGCCACATGTAGAAAGAT | |
| ATCTTCCAAAACGAAATCCTCCAGCATCATTGGATCCAGTTCAGC | |
| CTCACATACTTCTCAAGCGACATCAGGAGCCAATAGCAAATTGG | |
| GGATTATGGCTCCACCGAAGCCTATAAATAGACCGTTTCTTAAGC | |
| CTTCATATGCATTCT (SEQ ID NO: 160) | |
| Fragment 5: | TK PolyA |
| CATAAGGGGGAGGCTAACTGAAACACGGAAGGAGACAATACCG | F1 Origin |
| GAAGGAACCCGCGCTATGACGGCAATAAAAAGACAGAATAAAA | SV40 |
| CGCACGGGTGTTGGGTCGTTTGTTCATAAACGCGGGGTTCGGTCC | Promoter |
| CAGGGCTGGCACTCTGTCGATACCCCACCGAGACCCCATTGGGG | |
| CCAATACGCCCGCGTTTCTTCCTTTTCCCCACCCCACCCCCCAAG | |
| TTCGGGTGAAGGCCCAGGGCTCGCAGCCAACGTCGGGGCGGCAG | |
| GCCCTGCCATAGCAGATCTGCGCAGCTGGGGCTCTAGGGGGTAT | |
| CCCCACGCGCCCTGTAGCGGCGCATTAAGCGCGGCGGGTGTGGT | |
| GGTTACGCGCAGCGTGACCGCTACACTTGCCAGCGCCCTAGCGC | |
| CCGCTCCTTTCGCTTTCTTCCCTTCCTTTCTCGCCACGTTCGCCGG | |
| CTTTCCCCGTCAAGCTCTAAATCGGGGCATCCCTTTAGGGTTCCG | |
| ATTTAGTGCTTTACGGCACCTCGACCCCAAAAAACTTGATTAGGG | |
| TGATGGTTCACGTAGTGGGCCATCGCCCTGATAGACGGTTTTTCG | |
| CCCTTTGACGTTGGAGTCCACGTTCTTTAATAGTGGACTCTTGTT | |
| CCAAACTGGAACAACACTCAACCCTATCTCGGTCTATTCTTTTGA | |
| TTTATAAGGGATTTTGGGGATTTCGGCCTATTGGTTAAAAAATGA | |
| GCTGATTTAACAAAAATTTAACGCGAATTAATTCTGTGGAATGTG | |
| TGTCAGTTAGGGTGTGGAAAGTCCCCAGGCTCCCCAGCAGGCAG | |
| AAGTATGCAAAGCATGCATCTCAATTAGTCAGCAACCAGGTGTG | |
| GAAAGTCCCCAGGCTCCCCAGCAGGCAGAAGTATGCAAAGCATG | |
| CATCTCAATTAGTCAGCAACCATAGTCCCGCCCCTAACTCCGCCC | |
| ATCCCGCCCCTAACTCCGCCCAGTTCCGCCCATTCTCCGCCCCAT | |
| GGCTGACTAATTTTTTTTATTTATGCAGAGGCCGAGGCCGCCTCT | |
| GCCTCTGAGCTATTCCAGAAGTAGTGAGGAGGCTTTTTTGGAGG | |
| CCTAGGCTTTTGCAAAAAGCTCCCGGGAGCTTGTATATCCATTTT | |
| CGGATCTGATCAAGAGACAGGATGAGGATCGTTTCGCATGATTG | |
| AACAAGATGGATTGCACGCAGGTTCTCCGGCCGCTTGGGTGGAG | |
| AGGCTATTCGGCTATGACTGGGCACAACAGACAATCGGCTGCTC | |
| TGATGCCGCCGTGTTCCGGCTGTCAGCGCAGGGGCGCCCGGTTCT | |
| TTTTGTCAAGACCGACCTGTCCGGTGCCCTGAATGAACTGCAGG | |
| ACGAGGCAGCGCGGCTATCGTGGCTGGCCACGACGGGCGTTCCT | |
| TGCGCAGCTGTGCTCGACGTTGTCACTGAAGCGGGAAGGGACTG | |
| GCTGCTATTGGGCGAAGTGCCGGGGCAGGATCTCCTGTCATCTC | |
| ACCTTGCTCCTGCCGAGAAAGTATCCATCATGGCTGATGCAATGC | |
| GGCGGCTGCATACGCTTGATCCGGCTACCTGCCCATTCGACCACC | |
| AAGCGAAACATCGCATCGAGCGAGCACGTACTCGGATGGAAGCC | |
| GGTCTTGTCGATCAGGATGATCTGGACGAAGAGCATCAGGGGCT | |
| CGCGCCAGCCGAACTGTTCGCCAGGCTCAAGGCGCGCATGCCCG | |
| ACGGCGAGGATCTCGTCGTGACCCATGGCGATGCCTGCTTGCCG | |
| AATATCATGGTGGAAAATGGCCGCTTTTCTGGATTCATCGACTGT | |
| GGCCGGCTGGGTGTGGCGGACCGCTATCAGGACATAGCGTTGGC | |
| TACCCGTGATATTGCTGAAGAGCTTGGCGGCGAATGGGCTGACC | |
| GCTTCCTCGTGCTTTACGGTATCGCCGCTCCCGATTCGCAGCGCA | |
| TCGCCTTCTATCGCCTTCTTGACGAGTTCTTCTGAATGGGGATA | |
| (SEQ ID NO: 161) | |
| Fragment 6: | hExo |
| CAGGGATTGCTACAATTTATCAAAGAAGCTTCAGAACCCATCCA | pUC Origin |
| TGTGAGGAAGTATAAAGGGCAGGTAGTAGCTGTGGATACATATT | AmpR |
| GCTGGCTTCACAAAGGAGCTATTGCTTGTGCTGAAAAACTAGCC | |
| AAAGGTGAACCTACTGATAGGTATGTAGGATTTTGTATGAAATTT | |
| GTAAATATGTTACTATCTCATGGGATCAAGCCTATTCTCGTATTT | |
| GATGGATGTACTTTACCTTCTAAAAAGGAAGTAGAGAGATCTAG | |
| AAGAGAAAGACGACAAGCCAATCTTCTTAAGGGAAAGCAACTTC | |
| TTCGTGAGGGGAAAGTCTCGGAAGCTCGAGAGTGTTTCACCCGG | |
| TCTATCAATATCACACATGCCATGGCCCACAAAGTAATTAAAGC | |
| TGCCCGGTCTCAGGGGGTAGATTGCCTCGTGGCTCCCTATGAAGC | |
| TGATGCGCAGTTGGCCTATCTTAACAAAGCGGGAATTGTGCAAG | |
| CCATAATTACAGAGGACTCGGATCTCCTAGCTTTTGGCTGTAAAA | |
| AGGTAATTTTAAAGATGGACCAGTTTGGAAATGGACTTGAAATT | |
| GATCAAGCTCGGCTAGGAATGTGCAGACAGCTTGGGGATGTATT | |
| CACGGAAGAGAAGTTTCGTTACATGTGTATTCTTTCAGGTTGTGA | |
| CTACCTGTCATCACTGCGTGGGATTGGATTAGCAAAGGCATGCA | |
| AAGTCCTAAGACTAGCCAATAATCCAGATATAGTAAAGGTTATC | |
| AAGAAAATTGGACATTATCTCAAGATGAATATCACGGTACCAGA | |
| GGATTACATCAACGGGTTTATTCGGGCCAACAATACCTTCCTCTA | |
| TCAGCTAGTTTTTGATCCCATCAAAAGGAAACTTATTCCTCTGAA | |
| CGCCTATGAAGATGATGTTGATCCTGAAACACTAAGCTACGCTG | |
| GGCAATATGTTGATGATTCCATAGCTCTTCAAATAGCACTTGGAA | |
| ATAAAGATATAAATACTTTTGAACAGATCGATGACTACAATCCA | |
| GACACTGCTATGCCTGCCCATTCAAGAAGTCGTAGTTGGGATGA | |
| CAAAACATGTCAAAAGTCAGCTAATGTTAGCAGCATTTGGCATA | |
| GGAATTACTCTCCCAGACCAGAGTCGGGTACTGTTTCAGATGCCC | |
| CACAATTGAAGGAAAATCCAAGTACTGTGGGAGTGGAACGAGTG | |
| ATTAGTACTAAAGGGTTAAATCTCCCAAGGAAATCATCCATTGT | |
| GAAAAGACCAAGAAGTGCAGAGCTGTCAGAAGATGACCTGTTG | |
| AGTCAGTATTCTCTTTCATTTACGAAGAAGACCAAGAAAAATAG | |
| CTCTGAAGGCAATAAATCATTGAGCTTTTCTGAAGTGTTTGTGCC | |
| TGACCTGGTAAATGGACCTACTAACAAAAAGAGTGTAAGCACTC | |
| CACCTAGGACGAGAAATAAATTTGCAACATTTTTACAAAGGAAA | |
| AATGAAGAAAGTGGTGCAGTTGTGGTTCCAGGGACCAGAAGCAG | |
| GTTTTTTTGCAGTTCAGATTCTACTGACTGTGTATCAAACAAAGT | |
| GAGCATCCAGCCTCTGGATGAAACTGCTGTCACAGATAAAGAGA | |
| ACAATCTGCATGAATCAGAGTATGGAGACCAAGAAGGCAAGAG | |
| ACTGGTTGACACAGATGTAGCACGTAATTCAAGTGATGACATTC | |
| CGAATAATCATATTCCAGGTGATCATATTCCAGACAAGGCAACA | |
| GTGTTTACAGATGAAGAGTCCTACTCTTTTAAGAGCAGCAAATTT | |
| ACAAGGACCATTTCACCACCCACTTTGGGAACACTAAGAAGTTG | |
| TTTTAGTTGGTCTGGAGGTCTTGGAGATTTTTCAAGAACGCCGAG | |
| CCCCTCTCCAAGCACAGCATTGCAGCAGTTCCGAAGAAAGAGCG | |
| ATTCCCCCACCTCTTTGCCTGAGAATAATATGTCTGATGTGTCGC | |
| AGTTAAAGAGCGAGGAGTCCAGTGACGATGAGTCTCATCCCTTA | |
| CGAGAAGGGGCATGTTCTTCACAGTCCCAGGAAAGTGGAGAATT | |
| CTCACTGCAGAGTTCAAATGCATCAAAGCTTTCTCAGTGCTCTAG | |
| TAAGGACTCTGATTCAGAGGAATCTGATTGCAATATTAAGTTACT | |
| TGACAGTCAAAGTGACCAGACCTCCAAGCTATGTTTATCTCATTT | |
| CTCAAAAAAAGACACACCTCTAAGGAACAAGGTTCCTGGGCTAT | |
| ATAAGTCCAGTTCTGCAGACTCTCTTTCTACAACCAAGATCAAAC | |
| CTCTAGGACCTGCCAGAGCCAGTGGGCTGAGCAAGAAGCCGGCA | |
| AGCATCCAGAAGAGAAAGCATCATAATGCCGAGAACAAGCCGG | |
| GGTTACAGATCAAACTCAATGGAGCTCTGGAAAAACTTTGGATT | |
| TAAGCGGGACTCTGGGGTTCGCGAAATGACCGACCAAGCGACGC | |
| CCAACCTGCCATCACGAGATTTCGATTCCACCGCCGCCTTCTATG | |
| AAAGGTTGGGCTTCGGAATCGTTTTCCGGGACGCCGGCTGGATG | |
| ATCCTCCAGCGCGGGGATCTCATGCTGGAGTTCTTCGCCCACCCC | |
| AACTTGTTTATTGCAGCTTATAATGGTTACAAATAAAGCAATAGC | |
| ATCACAAATTTCACAAATAAAGCATTTTTTTCACTGCATTCTAGT | |
| TGTGGTTTGTCCAAACTCATCAATGTATCTTATCATGTCTGTATA | |
| CCGTCGACCTCTAGCTAGAGCTTGGCGTAATCATGGTCATAGCTG | |
| TTTCCTGTGTGAAATTGTTATCCGCTCACAATTCCACACAACATA | |
| CGAGCCGGAAGCATAAAGTGTAAAGCCTGGGGTGCCTAATGAGT | |
| GAGCTAACTCACATTAATTGCGTTGCGCTCACTGCCCGCTTTCCA | |
| GTCGGGAAACCTGTCGTGCCAGCTGCATTAATGAATCGGCCAAC | |
| GCGCGGGGAGAGGCGGTTTGCGTATTGGGCGCTCTTCCGCTTCCT | |
| CGCTCACTGACTCGCTGCGCTCGGTCGTTCGGCTGCGGCGAGCG | |
| GTATCAGCTCACTCAAAGGCGGTAATACGGTTATCCACAGAATC | |
| AGGGGATAACGCAGGAAAGAACATGTGAGCAAAAGGCCAGCAA | |
| AAGGCCAGGAACCGTAAAAAGGCCGCGTTGCTGGCGTTTTTCCA | |
| TAGGCTCCGCCCCCCTGACGAGCATCACAAAAATCGACGCTCAA | |
| GTCAGAGGTGGCGAAACCCGACAGGACTATAAAGATACCAGGC | |
| GTTTCCCCCTGGAAGCTCCCTCGTGCGCTCTCCTGTTCCGACCCT | |
| GCCGCTTACCGGATACCTGTCCGCCTTTCTCCCTTCGGGAAGCGT | |
| GGCGCTTTCTCAATGCTCACGCTGTAGGTATCTCAGTTCGGTGTA | |
| GGTCGTTCGCTCCAAGCTGGGCTGTGTGCACGAACCCCCCGTTCA | |
| GCCCGACCGCTGCGCCTTATCCGGTAACTATCGTCTTGAGTCCAA | |
| CCCGGTAAGACACGACTTATCGCCACTGGCAGCAGCCACTGGTA | |
| ACAGGATTAGCAGAGCGAGGTATGTAGGCGGTGCTACAGAGTTC | |
| TTGAAGTGGTGGCCTAACTACGGCTACACTAGAAGGACAGTATT | |
| TGGTATCTGCGCTCTGCTGAAGCCAGTTACCTTCGGAAAAAGAG | |
| TTGGTAGCTCTTGATCCGGCAAACAAACCACCGCTGGTAGCGGT | |
| GGTTTTTTTGTTTGCAAGCAGCAGATTACGCGCAGAAAAAAAGG | |
| ATCTCAAGAAGATCCTTTGATCTTTTCTACGGGGTCTGACGCTCA | |
| GTGGAACGAAAACTCACGTTAAGGGATTTTGGTCATGAGATTAT | |
| CAAAAAGGATCTTCACCTAGATCCTTTTAAATTAAAAATGAAGTT | |
| TTAAATCAATCTAAAGTATATATGAGTAAACTTGGTCTGACAGTT | |
| ACCAATGCTTAATCAGTGAGGCACCTATCTCAGCGATCTGTCTAT | |
| TTCGTTCATCCATAGTTGCCTGACTCCCCGTCGTGTAGATAACTA | |
| CGATACGGGAGGGCTTACCATCTGGCCCCAGTGCTGCAATGATA | |
| CCGCGAGACCCACGCTCACCGGCTCCAGATTTATCAGCAATAAA | |
| CCAGCCAGCCGGAAGGGCCGAGCGCAGAAGTGGTCCTGCAACTT | |
| TATCCGCCTCCATCCAGTCTATTAATTGTTGCCGGGAAGCTAGAG | |
| TAAGTAGTTCGCCAGTTAATAGTTTGCGCAACGTTGTTGCCATTG | |
| CTACAGGCATCGTGGTGTCACGCTCGTCGTTTGGTATGGCTTCAT | |
| TCAGCTCCGGTTCCCAACGATCAAGGCGAGTTACATGATCCCCC | |
| ATGTTGTGCAAAAAAGCGGTTAGCTCCTTCGGTCCTCCGATCGTT | |
| GTCAGAAGTAAGTTGGCCGCAGTGTTATCACTCATGGTTATGGC | |
| AGCACTGCATAATTCTCTTACTGTCATGCCATCCGTAAGATGCTT | |
| TTCTGTGACTGGTGAGTACTCAACCAAGTCATTCTGAGAATAGTG | |
| TATGCGGCGACCGAGTTGCTCTTGCCCGGCGTCAATACGGGATA | |
| ATACCGCGCCACATAGCAGAACTTTAAAAGTGCTCATCATTGGA | |
| AAACGTTCTTCGGGGCGAAAACTCTCAAGGATCTTACCGCTGTTG | |
| AGATCCAGTTCGATGTAACCCACTCGTGCACCCAACTGATCTTCA | |
| GCATCTTTTACTTTCACCAGCGTTTCTGGGTGAGCAAAAACAGGA | |
| AGGCAAAATGCCGCAAAAAAGGGAATAAGGGCGACACGGAAAT | |
| GTTGAATACTCATACTCTTCCTTTTTCAATATTATTGAAGCATTTA | |
| TCAGGGTTATTGTCTCATGAGCGGATACATATTTGAATGTATTTA | |
| GAAAAATAAACAAATAGGGGTTCCGCGCACATTTCCCCGAAAAG | |
| TGCCACCTGACGTCGACGGATCGGGAGATCTCCCGATCCCCTAT | |
| GGTCGACTCTCAGTACAATCTGCTCTGATGCCGCATAGTTAAGCC | |
| AGTATCTGCTCCCTGCTTGTGTGTTGGAGGTCGCTGAGTAGTGCG | |
| CGAGCAAAATTTAAGCTACAACAAGGCAAGGCTTGACCGACAAT | |
| TGCATGAAGAATCTGCTTAGG (SEQ ID NO: 162) | |

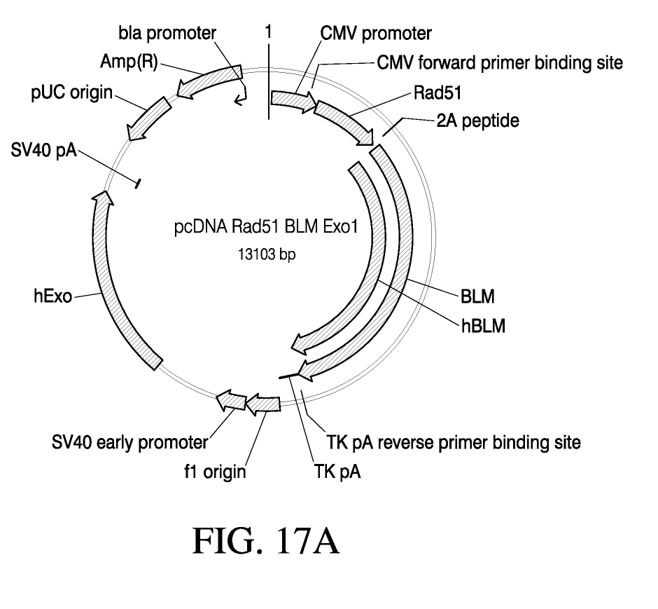

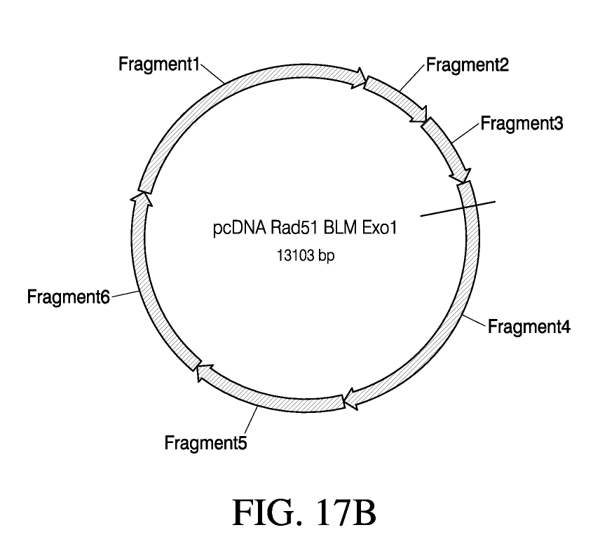

Table 14 shows the nucleotide sequence of the pcDNA Rad51 BLM Exo1 vector. Also, indicated in Table 14 are the nucleotide sequences of a number of vector elements. As shown in FIG. 17A-7B and in Table 14, a number of the vector element are partially encoded by different fragments/segments to are assembled to generate a replicable vector.

Embodiments of apparatuses, systems and methods for providing a simplified workflow for nucleic acid sequencing are described in this specification. The section headings used herein are for organizational purposes only and are not to be construed as limiting the described subject matter in any way.

While the foregoing embodiments have been described in some detail for purposes of clarity and understanding, it will be clear to one skilled in the art from a reading of this disclosure that various changes in form and detail can be made without departing from the true scope of the embodiments disclosed herein. For example, all the techniques, apparatuses, systems and methods described above can be used in various combinations.

1. A method for covalently linking two nucleic acid segments, the method comprising:
(a) incubating the two nucleic acid segments with an exonuclease under conditions that allow for digestion of termini of the two nucleic acid segments to form complementary single-stranded regions on each nucleic acid segment and hybridization of the complementary single-stranded regions, wherein each of the two nucleic acid segments comprises three regions: a first exonuclease sensitive region, an exonuclease resistant region, and a second exonuclease sensitive region, wherein the second exonuclease sensitive region (i) is located at one of the termini of each of the two nucleic acid segments, (ii) is from 5 to 30 base pairs in length, and (iii) forms the complementary single-stranded regions on each nucleic acid segment, and
(b) covalently connecting at least one strand of the hybridized termini formed in (a) resulting in the linkage of the two nucleic acid segments.

2. The method of claim 1, wherein steps (a) and (b) occur in the same tube.

3. The method of claim 1, wherein steps (a) and (b) occur in the same reaction mixture.

4. The method of claim 1, wherein the two or more nucleic acid segments are simultaneously contacted with an exonuclease and a ligase in step (a).

5. The method of claim 1, wherein the covalently linking of at least one strand of the hybridized termini formed in (a) is mediated by a ligase.

6. The method of claim 1, wherein three or more nucleic acid segments are covalently linked to each other.

7. The method of claim 1, wherein the two or more nucleic acid segments are covalently linked to one or more additional nucleic acid segments that do not contain exonuclease resistant regions.

8. The method of claim 1, wherein a replicable nucleic acid molecule is formed.

9. The method of claim 8, wherein the two or more nucleic acid segments are covalently linked form a circular nucleic acid molecule.

10. The method of claim 9, where the circular nucleic acid molecule contains one or more selection marker or origin of replication that is reconstituted by the linking of different nucleic acid segments.

11. The method of claim 9, wherein the circular nucleic acid molecule if formed from at least three nucleic acid segments.

12. The method of claim 8, wherein one or more enzyme contacting the nucleic acid molecule is partially or fully inactivated.

13. The method of claim 10, wherein inactivation of the one or more enzyme is inactivated by heating.

14. The method of claim 12, wherein the nucleic acid molecule is stored at −20° C. for at least two weeks after inactivation of the one or more enzyme.

15. A method for assembling a nucleic acid molecule, the method comprising:
(a) incubating a first nucleic acid segment with an exonuclease under conditions that allow for partial digestion of at least one terminus of the first nucleic acid segment to form a single-stranded region, wherein the first nucleic acid segment comprises three regions: a first exonuclease sensitive region, an exonuclease resistant region, and a second exonuclease sensitive region, wherein the second exonuclease sensitive region (i) is located at a terminus of the first nucleic acid segment, (ii) is from 5 to 30 base pairs in length, and (iii) forms the complementary single-stranded region,
(b) preparing a reaction mixture containing the digested first nucleic acid segment formed in (a) with a second nucleic acid segment under conditions that allow for the hybridization of termini with sequence complementarity, and
(c) covalently connecting at least one strand of the hybridized termini formed in (b).

16. The method of claim 15, wherein the second nucleic acid segment of (b) contains no exonuclease resistant regions.

17. The method of claim 15, wherein at least one terminus of the second nucleic acid segment of (b) contains a single-stranded region with sequence complementarity to the single-stranded region of the first nucleic acid molecules formed in step (a).

18. The method of claim 15, wherein the exonuclease is a 5′ to 3′ exonuclease.

19. The method of claim 15, wherein two or more exonucleases are present in step (a).

20. (canceled)

21. A method for assembling a nucleic acid molecule, the method comprising:
(a) incubating two or more nucleic acid segments with an exonuclease under conditions that allow for partial digestion of at least one terminus of each of the two or more nucleic acid segments to generate single-stranded termini, wherein at least two of the two or more nucleic acid segments comprise three regions: a first exonuclease sensitive region, an exonuclease resistant region, and a second exonuclease sensitive region, wherein the second exonuclease sensitive region (i) is located at one of the termini of each of the two nucleic acid segments, (ii) is from 5 to 30 base pairs in length, and (iii) forms the single-stranded termini,
(b) preparing a reaction mixture containing the digested nucleic acid segments prepared in (a) with one or more undigested nucleic acid segment under conditions that allow for the hybridization of termini with sequence complementarity, wherein at least one of the one or more undigested nucleic acid segment has region of sequence complementarity with at least one single-stranded terminus formed in (a), and
(c) covalently connecting at least one strand of the hybridized termini formed in (b).
